(19) United States
(12) Patent Application Publication
(10) Pub. No.: US 2023/0257825 A1
(43) Pub. Date: Aug. 17, 2023

(54) BREAST CANCER BIOMARKERS AND METHODS OF USE

(71) Applicant: City of Hope, Duarte, CA (US)

(72) Inventors: Peter P. Lee, San Marino, CA (US); Colt Egelston, Duarte, CA (US); Weihua Guo, Duarte, CA (US); Jiayi Tan, Duarte, CA (US)

(21) Appl. No.: 18/106,248

(22) Filed: Feb. 6, 2023

Related U.S. Application Data
(60) Provisional application No. 63/306,648, filed on Feb. 4, 2022.

(51) Int. Cl. C12Q 1/6886; G01N 33/569; G01N 33/574; A61K 45/06

(52) U.S. Cl. CPC C12Q 1/6886; G01N 33/56972; G01N 33/57415; A61K 45/06; C12Q 2600/158; G01N 2333/70514; G01N 2333/70517; G01N 2333/70592

(57) ABSTRACT

The disclosure provides, inter alia, breast cancer biomarkers, methods of detecting exhausted CD8+ T cells in breast cancer patients, methods of detecting CD26+CD4+ T cells in breast cancer patients, methods of treating breast cancer patients, and methods of identifying risk outcomes for breast cancer patients.

### CROSS-REFERENCE TO RELATED APPLICATIONS

This application claims the benefit of priority to U.S. Application No. 63/306,648 filed Feb. 4, 2022, the disclosure of which is incorporated by reference herein in its entirety.

### STATEMENT AS TO RIGHTS TO INVENTIONS MADE UNDER FEDERALLY SPONSORED RESEARCH AND DEVELOPMENT

This invention was made with Government support under Grant Number P30CA033572, awarded by the National Institutes of Health. The Government has certain rights in the invention.

### BACKGROUND

In most cancer types, presence of tumor infiltrating lymphocytes (TILs) denotes reduced risk for relapse and increased overall survival. The prognostic impact of CD8⁺ TILs appears to be subtype specific in breast cancer (BC). CD8⁺ tumor infiltrating T cells positively associate with survival in triple negative breast cancer (TNBC) and HER2/neu over-expressed (HER2+) BC, but not in estrogen receptor-positive (ER+) BC. This paradox is further complicated by differences in kinetics of disease progression amongst BC subtypes. As compared to TNBC patients, ER+BC patients rarely have early relapse events, but have a higher overall relapse rate when extending more than five years post diagnosis. A better understanding of the relationship between TILs and patient outcomes is needed to guide therapeutic strategies for ER+BC patients, which compose approximately 70% of all BC patients.

Progress in dissecting the complexity of CD8⁺ TIL heterogeneity has shed light on the role of specific T cell subsets in anti-tumor immunity. We and others have shown that primary tumor infiltrating resident memory T cells, a subset of CD8⁺ T cells that localizes within peripheral tissue without recirculation, positively associate with increased survival in TNBC patients. Tumor infiltration of granzyme B⁺ CD8⁺ TILs and an interferon-γ (IFNγ) signature also denote favorable outcomes in TNBC patients. However, a detailed understanding of the relationship between CD8⁺ TIL subsets and ER+BC patient survival characteristics is still lacking. The inventors have made important and unexpected findings on this relationship, such that the disclosure is directed to understanding and solving these need in the art, thereby providing better and more successful treatment options for patients with breast cancer.

### SUMMARY

Provided herein are methods of identifying a breast cancer patient at increased risk for relapse or reduced survival, the method comprising detecting an elevated level of exhausted CD8+ T cells, relative to a standard control, in a biological sample obtained from the patient, wherein the elevated level of exhausted CD8+ T cells indicates the patient is at increased risk for relapse or reduced survival. In embodiments, the elevated level of exhausted CD8+ T cells comprises an unequally weighted average of the elevated gene expression levels of the genes in Table 1.

Provided herein are methods of detecting an elevated level of exhausted CD8+ T cells in a breast cancer patient, the method comprising detecting an elevated level of exhausted CD8+ T cells, relative to a standard control, in a biological sample obtained from the breast cancer patient. In embodiments, the elevated level of exhausted CD8+ T cells comprises an unequally weighted average of the elevated gene expression levels of the genes in Table 1.

Provided herein are methods of treating breast cancer in a patient in need thereof, the method comprising: (i) detecting an elevated level of exhausted CD8+ T cells, relative to a standard control, in a biological sample obtained from the patient; and (ii) administering to the patient an effective amount of a chemotherapeutic therapy. In embodiments, the elevated level of exhausted CD8+ T cells comprises an unequally weighted average of the elevated gene expression levels of the genes in Table 1. In embodiments, the chemotherapeutic therapy comprises a chemotherapeutic agent and optionally further comprises a non-chemotherapeutic agent, such as hormone therapy, immunotherapy, targeted therapy, radiation, gene therapy, or a combination thereof.

Provided herein are methods of identifying a breast cancer patient at increased risk for relapse or reduced survival, the method comprising detecting a decreased level of CD26+CD4+ T cells, relative to a control, in a biological sample obtained from the patient. In embodiments, the decreased level of CD26+CD4+ T cells comprises an unequally weighted average of the decreased gene expression levels of the genes in Table 2.

Provided herein are methods of detecting a decreased level of CD26+CD4+ T cells in a breast cancer patient, the method comprising detecting a decreased level of CD26+CD4+ T cells, relative to a control, in a biological sample obtained from the breast cancer patient. In embodiments, the decreased level of CD26+CD4+ T cells comprises an unequally weighted average of the decreased gene expression levels of the genes in Table 2.

Provided herein are methods of treating breast cancer in a patient in need thereof, the method comprising: (i) detecting a decreased level of CD26+CD4+ T cells, relative to a control, in a biological sample obtained from the patient; and (ii) administering to the patient an effective amount of a chemotherapeutic therapy. In embodiments, the decreased level of CD26+CD4+ T cells comprises an unequally weighted average of the decreased gene expression levels of the genes in Table 2. In embodiments, the chemotherapeutic therapy comprises a chemotherapeutic agent and optionally further comprises a non-chemotherapeutic agent, such as hormone therapy, immunotherapy, targeted therapy, radiation, gene therapy, or a combination thereof.

Provided herein are methods of identifying a breast cancer patient having a good prognosis, the method comprising detecting an increased level of CD26+CD4+ T cells, relative to a standard control, in a biological sample obtained from the patient; wherein the increased level of CD26+CD4+ T cells indicates a good prognosis. In embodiments, the increased level of CD26+CD4+ T cells comprises an unequally weighted average of the increased gene expression levels of the genes in Table 2.

Provided herein are methods of detecting an increased level of CD26+CD4+ T cells in a breast cancer patient, the method comprising detecting an increased level of CD26+CD4+ T cells, relative to a standard control, in a biological sample obtained from the breast cancer patient. In embodiments, the increased level of CD26+CD4+ T cells comprises an unequally weighted average of the increased gene expression levels of the genes in Table 2.

Provided herein are methods of treating breast cancer in a patient in need thereof, the method comprising: (i) detecting an increased level of CD26+CD4+ T cells, relative to a standard control, in a biological sample obtained from the patient; and (ii) administering to the patient an effective amount of a non-chemotherapeutic therapy. In embodiments, the increased level of CD26+CD4+ T cells comprises an unequally weighted average of the increased gene expression levels of the genes in Table 2. In embodiments, the non-chemotherapeutic therapy comprises hormone therapy, immunotherapy, targeted therapy, radiation, gene therapy, or a combination of two or more thereof.

These and other embodiments of the disclosure are described in detail herein.

### BRIEF DESCRIPTION OF THE DRAWINGS

FIGS. 1A-1D show PD-1⁺ CD39⁺ CD8⁺ T cells in breast cancer patient tissues. FIG. 1A: Single cell suspensions from peripheral blood mononuclear cells (PBMCs), tumor negative lymph nodes (T− LN), tumor positive lymph nodes (T+LN), tumor, and non-cancerous breast tissue (NCBT) were examined by flow cytometry for expression of PD-1 and CD39 amongst antigen experienced (CD45RA⁻) CD8⁺ T cells. FIG. 1B: Frequencies of PD-1⁺ CD39⁺ cells within CD45RA⁻ CD8⁺ T cells in various tissues are shown (PBMC n=30, T-LN n=21, T+LN n=24, Tumor n=77, NCBT n=32). FIG. 1C: PD-1⁺ CD39⁺ frequencies are displayed within triple negative breast cancer (TNBC) and estrogen receptor-positive (ER+) tumors (TNBC n=11, ER+n=66). FIG. 1D: Experimental workflow for analysis of CD8⁺ T cells and patient tissues. Statistics generated by one-way ANOVA with Holm-Sidak multiple comparisons test (FIG. 1B) or unpaired t-tests (FIGS. 1C-1D)). *, p<0.05; ***, p<0.001.

FIGS. 2A-2K provide a phenotypic characterization of PD-1⁺ CD39⁺ CD8⁺ T cells in breast tumors. CD8⁺ TILs from patient tumors were examined by flow cytometry for expression of various proteins. FIG. 2A: Normalized PD-1 expression of PD-1⁺ CD39⁻ (orange) and PD-1⁺ CD39⁺ (red) CD8⁺ TILs, calculated by subtracting MFI values of PD-1⁻ CD8⁺ TILs in the same sample, (n=45). Frequencies of PD-1⁻ CD39⁻, PD-1⁺ CD39⁺, and PD-1⁺ CD39⁺ (red) CD8⁺ TILs expressing FIG. 2B, TIM-3 (n=25) FIG. 2C, TIGIT (n=14), FIG. 2D, B4 (n=33) FIG. 2E, CD38 (n=15) FIG. 2F, CD69 (n=33) FIG. 2G, CD103 (n=36). Frequencies of PD-1⁻ CD39⁻, PD-1⁺ CD39⁺, and PD-1⁺ CD39⁺ CD8⁺ TILs populations producing FIG. 211, IFNγ FIG. 21, TNFα, and FIG. 2J, IL-2, (n=29) after stimulation with PMA and ionomycin. FIG. 2K, Frequencies of each cell population for CD127 and KLRG1 expression profiles (n=30). All data was collected from 39 ER+ primary tumors and 6 TNBC primary tumors. Statistics generated by unpaired t-tests (FIG. 2A) or one-way ANOVA with Holm-Sidak multiple comparisons test (FIGS. 2B-2K). *, p<0.05; **, p<0.01; * p<0.001; ****, p<0.0001.

FIGS. 3A-3G show transcriptional features of CD8⁺ T_{EX} in breast tumors. CD8⁺ T cells from 8 ER+BC and 2 TNBC patient tissues were single cell index sorted for whole transcriptome analysis in the context of several cell surface proteins. FIG. 3A, tSNE projection of four major clusters of CD8⁺ T cells identified and annotated as exhausted T cells, resident effector memory T cells, effector memory T cells, and central memory T cells. FIG. 3B, Top 10 most differentially expressed genes for each CD8⁺ T cell cluster. FIG. 3C, tSNE overlay of CD8⁺ T cells identified as PD-1⁻ CD39⁻ (blue), PD-1⁺ CD39⁻ (orange), PD-1⁺ CD39⁺ (red), or PD-1-CD39⁺ (gray). FIG. 3D, Genes most significantly differentially expressed by PD-1⁺ CD39⁺ CD8⁺ T cells. FIG. 3E, Overlay of cell surface protein expression onto tSNE cluster projections. Protein expression for PD-1, CD103, CD69, CD39, CD137, and CCR7 were acquired from index sort information and shown here as positively (purple) or negatively (yellow) expressed for each cell. Gene Set Enrichment Analysis (GSEA) of PD-1⁺ CD39⁺ CD8⁺ T cell differentially expressed genes as compared to Tex signatures identified from FIG. 3F, lung cancer and FIG. 3G, melanoma publications. Gene rank shown is derived from the current dataset. (n=10 BC patients; 9 tumors; 2 T+LNs; 3 NCBTs; 7 matched PBMCs)

FIGS. 4A-4H show altered immune TME in T_{EX}hi breast tumors. ER+ breast tumors defined as T_{EX}hi (top 50%) or T_{EX}lo (bottom 50%) by flow cytometry were assayed by immunohistochemistry for CD8⁺ T cell infiltration (teal), CD20⁺ B cell infiltration (purple), and PD-L1 expression (brown). FIG. 4A, Representative high-powered fields (20×, scale bar=50 μm). Clinical pathologist scoring for FIG. 4B, CD8 FIG. 4C, CD20 and FIG. 4D, PD-L1 (T_{EX}hi n=18, T_{EX}lo n=18; unpaired t-test). TME features of ER+ breast tumors were assessed by Nanostring PanCancer Immune transcriptional profiling (n=36). FIG. 4E, Absolute abundance of cell type scores and FIG. 4F, inflammation related gene expression are displayed as heatmaps normalized across all tissues by cell type or gene (row). Tumor tissues (columns) are annotated by FACS T_{EX} frequency of CD8⁺ TILs normalized to % CD8 by IHC (% Tex; red), IHC CD8⁺ T cell infiltration score (% CD8; blue), and IHC PD-L1 expression score (% PD-L1; brown). FIG. 4G, Top 30 genes differentially expressed (uncorrected t-test p<0.01) between T_{EX}hi and T_{EX}lo tumors are shown. FIG. 4H, CIBERSORT analysis of relative immune populations in T_{EX}hi (top 25%, n=275) and T_{EX}lo (bottom 25%, n=275) ER+ breast tumors in METABRIC database. Statistics generated by Wilcoxon Rank Sum test. *, p<0.05; **, p<0.01; ***, p<0.001; ****, p<0.0001.

FIGS. 5A-5L show that T_{EX}hi tumors denote decreased survival in estrogen receptor positive breast cancer. METABRIC tumors were used to examine FIG. 5A, CD8A expression and FIG. 5B, T_{EX} expression in TNBC (green) and ER+(purple) tumors. Co-expression of CD8A and a T_{EX} signature in both FIG. 5C, TNBC and FIG. 5D, ER+ tumors. Tumors were stratified by CD8A expression into CD8hi (top 25%) and CD8lo (bottom 25%) groups to examine overall survival in FIG. 5E, TNBC and FIG. 5F, ER+BC. Similarly, tumors were stratified by T_{EX} signature expression into T_{EX}hi (top 25%) and T_{EX}lo (bottom 25%) groups to examine overall survival in FIG. 5G, TNBC and FIG. 511, ER+BC. Tumors were further stratified into cohort quartiles as CD8hiT_{EX}hi (top 50% T_{EX} of top 50% CD8), CD8hiT_{EX}lo (bottom 50% T_{EX} of top 50% CD8), CD8loT_{EX}hi (top 50% T_{EX} of bottom 50% CD8), and CD8loT_{EX}lo (bottom 50% T_{EX} of bottom 50% CD8) within FIG. 5I, TNBC and FIG. 5J, ER+ tumors. Influence of T_{EX} signature on overall survival in FIG. 5K, TNBC and FIG. 5L, ER+BC patients was compared to CD8A, CD3G, PTPRC (CD45) gene expression by multivariate Cox hazard ratio assessment. Statistics generated by unpaired student t-test (FIGS. 5A-5B), non-parametric Spearman rank correlation (FIGS. 5C-5D), log rank test (FIGS. 5E-5J), Wald test (FIGS. 5K-5L).

FIGS. 6A-6O show reduced overall survival in premenopausal women with estrogen receptor positive T_{EX}hi tumors. FIG. 6A, Differential expression of Hallmark pathway gene sets between T_{EX}hi and T_{EX}lo TCGA ER+ breast cancer tumors was performed. Normalized enrichment scores are shown for all pathways with p<0.05. Gene set enrichment for genes upregulated in T_{EX}hi tumors are shown for FIG. 6B, epithelial mesenchymal transition and FIG. 6C, estrogen response early pathways. FIG. 6D, METABRIC ER+ tumors were assessed for the T_{EX} signature in amongst tumor grades. FIG. 6E, T_{EX}hi and T_{EX}lo tumors were compared for a proliferation signature score. METABRIC defined postmenopausal ER+ patient tumors were stratified by T_{EX} signature expression into T_{EX}hi (top 25%) and T_{EX}lo (bottom 25%) groups to examine FIG. 6F, overall survival and FIG. 6G, relapse-free survival. FIG. 6H, Overall survival was also compared between CD8hiT_{EX}hi (top 50% T_{EX} of top 50% CD8), CD8hiT_{EX}lo (bottom 50% T_{EX} of top 50% CD8), CD8loT_{EX}hi (top 50% T_{EX} of bottom 50% CD8), and CD8loT_{EX}lo (bottom 50% T_{EX} of bottom 50% CD8). In the same way, FIG. 6I, overall survival and FIG. 6J, relapse-free survival was compared in premenopausal ER+ patient T_{EX}hi and T_{EX}lo groups. FIG. 6K, Overall survival in premenopausal CD8hiT_{EX}hi, CD8hiT_{EX}lo, CD8loT_{EX}hi, and CD8loT_{EX}lo subgroups. Premenopausal ER+BC patient tumor FIG. 6L, grade FIG. 6M, Pam50 molecular subset FIG. 6N, and proliferation signature score in T_{EX}hi and T_{EX}lo tumors. FIG. 6O, multivariate Cox hazard ratios for overall survival in relation to T_{EX} signature expression amongst varying age groups in ER+ and TNBC METABRIC patients. Statistics generated as noted or by one-way ANOVA with Holm-Sidak corrected multiple comparisons T test (FIG. 6D), by unpaired student t-test (FIGS. 6E, 6N), or by log rank test (FIGS. 6F-6K).

FIGS. 7A-7J show that a T_{EX}hi signature identifies reduced survival in premenopausal patients with intermediate Oncotype DX breast recurrence scores. A relative Oncotype DX breast recurrence score (BRS) was calculated for Metabric ER+ tumors. Tumors were classified as Oncotype DX BRS high (top 15%), low (bottom 15%), and intermediate (middle 70%). Postmenopausal patients within intermediate Oncotype DX BRS (Onc_{DX}int) patients were further stratified as T_{EX}hi (top 25%) and T_{EX}lo (bottom 25%) and examined for differences in FIG. 7A, overall survival FIG. 7B, relapse-free survival and FIG. 7C, influence on overall survival by multivariate Cox hazard ratio assessment. Similarly, premenopausal patients within Onc_{DX}int patients were further stratified as T_{EX}hi and T_{EX}lo and examined for differences in FIG. 7D, overall survival FIG. 7E, relapse-free survival and FIG. 7F, influence on overall survival by multivariate Cox hazard ratio assessment. Overall survival and relapse-free survival were then compared amongst postmenopausal (FIGS. 7G-7H) and premenopausal (FIGS. 7I-7J) ER+ patients between those defined as high Oncotype DX BRS (Onc_{DX}hi), Onc_{DX}int+T_{EX}lo, Onc_{DX}int+T_{EX}hi, and low Oncotype DX BRS (Onc_{DX}lo). Statistics generated by log rank test (FIGS. 7A-7B, 7D, 7E, 7G-7J). Cox hazard ratios were calculated using multivariate accounting for variables shown (FIGS. 7C, 7F).

FIG. 8 shows the gating strategy for CD8+ T cells prior to analysis of PD-1 and CD39 expressing subsets.

FIGS. 9A-9D show frequency of PD-1+CD39+ cells within CD8+ TILs in ER+ tumors. Frequencies of memory (CD45RA−) CD8+ TILs in ER+ tumors are stratified by FIG. 9A, Ki67 scoring FIG. 9B, tumor grade FIG. 9C, T status (tumor size) and FIG. 9D, patient staging (n=66).

FIGS. 10A-10I are representative flow cytometry plots for protein expression of CD8+ TIL subsets. CD8+ TTLs were gated for PD-1− CD39− (blue), PD-1+CD39− (orange), and PD-1+CD39+(red) subsets. These subsets were then assessed for expression of FIG. 10A, PD-1, FIG. 10B, TIM-3 FIG. 10C, TIGIT FIG. 10D, 2B4 FIG. 10E, CD38 FIG. 10F, CD69, and FIG. 10G, CD103. CD8+ TILs were stimulated with PMA and ionomycin and assessed for production of FIG. 10H, IFNγ, TNFα, and IL-2 by intracellular flow cytometry. CD8+ TILs were also assessed for markers of T cell differentiation i, CD127 and KLRG1.

FIGS. 11A-11C provide batch effect analysis of single cell cluster identification. tSNE projections of CD8+ T cells assayed by single cell sequencing are shown overlaid with FIG. 11A, tissue origin of cell FIG. 11B, patient ID and FIG. 11C, patient ID with each T cell subset overlaid individually. All patient tumors were ER+, except P379 and P365, which were TNBC.

FIGS. 12A-12B are Gene Set Enrichment Analysis of exhaustion signatures. Breast tumor Tex differentially expressed genes were compared to published gene expression data from two publications utilizing a LCMV murine model of chronic exhaustion: FIG. 12A, Schettinger, Immunity 2016 FIG. 12B, Wherry, Immunity 2007. Gene rank shown is from the current datase.

FIGS. 13A-13C show differential immune infiltration and gene expression in _{TEXhi} and _{TEXlo} tumors. ER+ breast tumors were assessed by Nanostring PanCancer Immune transcriptional profiling for correlations between FIG. 13A, absolute abundance of immune cell subsets or FIG. 13B, inflammation related genes and known abundance of CD8+_{TEX}, CD8+ T cells, or PD-L1 expression (n=36). Correlation coefficients (R) values and p values are shown. P value text is strikethrough if p>0.05. FIG. 13C, METABRIC ER+ tumors were examined for differential gene expression between Ex high (red; top 25%) and Ex low (blue; bottom 25%). Hallmark pathways of differentially expressed genes (p<0.05) are annotated as described. Selected genes upregulated in _{TEXhi} tumors are called out.

FIGS. 14A-14J show exhausted CD8+ T cell signature in breast cancer patients. FIG. 14A, CD8+ T cell exhaustion (TEX) signatures were assessed for their prevalence in METABRIC cohort tissues in all invasive breast cancers by PAM50 molecular signatures of breast cancer. Mutation burden, or number of detected mutations detected, are shown for _{TEX} high (red; top 25%) and _{TEX} low (blue; bottom 25%) in FIG. 14B, TNBC and FIG. 14C, ER+ METABRIC breast tumors. FIG. 14D, Tumor mutation load (Oncomine software derived score) was also assessed in the context of % Tex of CD8+ TILs as assessed by flow cytometry in ER+ breast tumors. CD8 cutoff groups and TEX cutoff groups (top 25%, bottom 25%) from ER+(FIGS. 14E-14F) and TNBC (FIGS. 1411-14I) METABRIC tumors are shown. Final four group composition of TEX hi and low (top 50%, bottom 50%) within CD8 hi and low (top 50%, bottom 50%) are shown for ER+(FIG. 14G) and TNBC (FIG. 14J) METABRIC tumors. Statistics were generated by one-way ANOVA (FIGS. 14A-14B), Wilcoxon Rank Sum test (FIGS. 14C-14D), Pearson's rank correlation (FIG. 14D), and non-parametric Spearman rank correlation (FIGS. 14E-14J).

FIGS. 15A-15D show exhausted CD8+ T cell signature in breast cancer patients. FIG. 15A, METABRIC ER+ tumors were examined for correlation of interferon gene expression signature and a mx gene signature expression. FIG. 15B, A volcano plot of differential gene expression with upregulated genes in _{TEXhi} tumors on the right, p-value cutoff of 0.01. Select interferon-β related genes are called out. FIG. 15C, Expression levels of IFNγ related genes in cancer cells within _{TEXhi}^{tumors}_{TEXlo} tumors, as determined by flow cytometry median, was assessed by single cell RNA sequencing. FIG. 15D, Cox hazard ratios for IFNγ related genes in regard to overall survival in METABRIC ER+BC patients. Statistics generated with non-parametric Spearman rank correlation (a), Deseq (b), and Wilcoxon rank sum test (c).

FIGS. 16A-16F show molecular and pathological features of ER+BC tumors as they associate with infiltration of CD8+ _{TEX}. ^{The} _{TEX} gene signature expression level was assessed in METABRIC ER+BC tumors within a, PAM50 molecular subsets b, tissue pathology progesterone receptor (PR) expression c, patient staging d, tumor size e, menopausal status and f, age. Statistics generated with one-way ANOVA (a-c, e) and non-parametric Spearman rank correlation (d, f).

FIGS. 17A-17H show expanded survival characteristics of premenopausal and postmenopausal ER+BC patients. METABRIC defined postmenopausal ER+ patient tumors were stratified by T_{EX} signature expression into _{TEXhi} (top 25%) and TEXlo (bottom 25%) groups to examine overall survival and relapse-free survival in FIGS. 17A-17B, postmenopausal and FIGS. 17C-17D, premenopausal BC patients with Grade 2/3 only tumors. Overall survival and relapse-free survival in FIGS. 17E-17F, postmenopausal and FIGS. 17G-17H, premenopausal BC patients with Stage I-III only tumors.

FIGS. 18A-18C show relationship between mx signature expression and Oncotype DX breast recurrence score in ER+ breast tumors. Relative Oncotype DX breast recurrence scores (BRS) and mx gene signature scores from METABRIC ER+BC tumors within FIG. 18A, all ER+ patients FIG. 18B, postmenopausal ER+ patients and FIG. 18C, premenopausal ER+ patients. Statistics generated with Pearson's correlation.

FIGS. 19A-19B provide multivariate regression model to assess the relationship of mx and survival in ER+ breast cancer patients. A hybrid version of stepwise model selection was used to prune the model so that only necessary covariates were included. Akaike Information Criterion, or AIC, was used to select the best fitted model. Forest plots of estimated hazard ratios were generated for final fitted models. Schoenfeld residual global p-values was used to test for proportional hazard assumption. Results are shown for influence of variables on FIG. 19A, overall survival and FIG. 19B, relapse=free survival.

FIG. 20 shows the clinical characteristics of breast cancer patient tumor samples for fresh tissue studies.

FIG. 21 shows the reagents used for flow cytometry.

FIG. 22 shows the 25 gene signature of CD8+T_{EX}.

FIGS. 23A-23D show CD26+CD4+ T cells in breast cancer patient tissues. FIG. 23A, Single cell suspensions from peripheral blood (PB), breast cancer (BC) tumor, normal breast tissue were examined by flow cytometry for expression of CD26 amongst CD4⁺ T cells. FIG. 23B, Frequencies of CD26⁺ cells within CD45RA⁻ CD8⁺ T cells in various tissues are shown. FIG. 23C, Representative plots of IFNγ, TNFα, and IL-2 expression in CD26 defined CD4⁺ T cell subsets. FIG. 23D, Frequency of IFNγ, TNFα, and IL-2 expression in CD26 defined CD4⁺ T cell subsets.

FIGS. 24A-24B show distinct gene expression of CD4⁺ T cells in breast cancer patient tumors. Single cell suspensions from breast cancer tumor were assayed by single cell RNA sequencing. FIG. 24A, UMAP plot of 11 unique identified CD4+ T cell subsets. FIG. 24B, Heatmap of differential gene expression in the 11 unique CD4+ T cell subsets.

FIGS. 25A-25D show tumors with high CD26 signature expression denote increased survival in breast cancer patients. METABRIC tumors were used to examine the association between a CD26 gene expression signature and survival in FIG. 25A, ER+ breast cancer patients and FIG. 25B, TNBC patients, and FIG. 25C, ER+ postmenopausal patients. Survival was then examined by FIG. 25D, a combined approach stratifying patients with both the CD26 gene expression signature and an exhausted T cell (Tex) signature.

FIGS. 26A-26E provide single-cell RNA-sequencing data quality control and principal component selection. Violin plot for number of non-zero expression genes for each cell, FIG. 26A. Violin plot for percentage of mitochondrial gene expression of each cell, FIG. 26B. Selected top 1,000 most variable genes (red dots) for PCA (top 20 are labeled with gene names), FIG. 26C. Modified Jack Straw method for PC selection, FIG. 26D. Elbow plot for PC selection (i.e., a ranking of principle components based on the percentage of variance explained by each one), FIG. 26E.

FIGS. 27A-27D shows the identification of exhausted CD8+ T cells via multiplex immunohistochemistry in human breast tumors. FIG. 27A is a representative composite image of a breast cancer patient tumor tissue stained for the presence of exhausted CD8+ T cells (see white arrows) defined by co-expression of CD3, CD8, and CXCL13. Immune markers are shown separately for CXCL13 (red) and pan-cytokeratin (CK, grey) (FIG. 27B); CD3 (orange) and CK (FIG. 27C); and CD8 (cyan) and CK (FIG. 27D). Tissues were stained via primary antibodies (CD3, polyclonal Dako; CD8, clone SP16 Biocare; CXCL13 polyclonal R&D), amplified via HRP conjugated secondary antibody (Biocare) plus opal-TSA hybridization (Akoya), and then imaged on a multispectral microscope (Akoya Vectra III).

FIGS. 28A-28D shows the identification of CD26+CD4+ T cells via multiplex immunohistochemistry in human breast tumors. FIG. 28A is a representative composite image of a breast cancer patient tumor tissue stained for the presence of CD26+CD4+ T cells (see white arrows) defined by co-expression of CD3 and CD26 and lack of CD8. Immune markers are shown separately for CD26 (red) and pan-cytokeratin (CK, grey) (FIG. 28B); CD3 (orange) and CK (FIG. 28C); and CD8 (cyan) and CK (FIG. 28D). Tissues were stained via primary antibodies (CD3, polyclonal Dako; CD8, clone SP16 Biocare; CD26 clone D6D8K Cell Signaling Technology), amplified via HRP conjugated secondary antibody (Biocare) plus opal-TSA hybridization (Akoya), and then imaged on a multispectral microscope (Akoya Vectra III).

FIG. 29 is a Kaplan-Meier curve showing the prognostic value of CD4+CD26+ T cells within OncotypeDx intermediate group (score 16-25) in ER+ breast cancer subtype post-menopausal patients from METABRIC dataset. The high and low groups of CD4+CD26+ were defined by top 25% and bottom 25% of CD4+CD26+ signature score.

FIG. 30 are Multivariate Cox hazard ratios for distal metastasis-free survival in relation to CD4+CD26+ signature score accounting for the influence of age, tumor grade, tumor size, and nodal status in OncotypeDx intermediate group (score 16-25), ER+ breast cancer subtype post-menopausal patients.

FIG. 31 is a Kaplan-Meier curve showing the prognostic value of CD4+CD26+ T cells within OncotypeDx low and intermediate groups (score 0-25) in ER+ breast cancer subtype post-menopausal patients from METABRIC dataset. The high and low groups of CD4+CD26+ were defined by top 25% and bottom 25% of CD4+CD26+ signature score.

FIG. 32 are Multivariate Cox hazard ratios for distal metastasis-free survival in relation to CD4+CD26+ signature score accounting for the influence of age, tumor grade, tumor size, and nodal status in OncotypeDx low and intermediate groups (score 0-25), ER+ breast cancer subtype post-menopausal patients.

### DETAILED DESCRIPTION

### Definitions

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, N.Y. 1994); Sambrook et al., Molecular Cloning, A Laboratory Manual, Cold Springs Harbor Press (Cold Springs Harbor, N Y 1989). Any methods, devices and materials similar or equivalent to those described herein can be used in the practice of this disclosure. The following definitions are provided to facilitate understanding of certain terms used frequently herein and are not meant to limit the scope of the present disclosure.

“T cells” are a type of lymphocyte that originate in the hematopoietic stem cells in the bone marrow. Exemplary T cells include CD4⁺ T cells, CD8 T⁺ cells, memory T cells, regulatory T cells, natural killer T cells, mucosal associated invariant T cells, and gamma delta T cells. In embodiments, the T cells are CD4⁺ T cells. In embodiments, the T cells are CD8⁺ T cells.

A “CD4+ T cell” or “CD4⁺ T lymphocyte” as referred to herein is a lymphocyte that expresses the CD4 glycoprotein on its surface. CD4 T cells include helper T cells, which are T cells that help orchestrate the immune response, including antibody responses and killer T cell responses. CD4 T cell precursors differentiate into one of several subtypes, including TH1 (type 1 helper T cell), TH2 (type 2 helper T cell), TH3 (T helper 3 cells), TH17 (T helper 17 cells) or TFH (Follicular B helper T cells). These subtypes of helper T cells are characterized by their secretion of different cytokines to facilitate different types of immune responses.

A “CD8+ T cell or “CD8+T lymphocyte” is a lymphocyte that expresses the CD8 glycoprotein on its surface. Examples of CD8 T cells include cytotoxic T cells and natural killer cells.

“Exhausted CD8+ T cell” refers to CD8+ T cells that have been exposed to persistent antigenic stimulation which induces a dysfunctional state in which the CD8+ T cells have poor effector function.

“CD26” is a cell surface glycoprotein with DPPI-IV activity in its extracellular domain and is expressed on a variety of cell types, including T cell, B cells, and others.

“CD26+CD8+ T cell” is a lymphocyte that expresses both the CD8 glycoprotein and the CD26 glycoprotein on its surface.

“PD-1+” refers to the programmed cell death 1 (PD-1) protein receptor that is expressed on the surface of T cells and B cells, PD-1 down-regulates the immune system and promotes self-tolerance by suppressing T cell inflammatory activity.

“Remission” means that the clinical signs and symptoms of breast cancer have been significantly diminished or have disappeared entirely based on clinical diagnostics, although cancerous cells can still exist in the body. Thus, it is contemplated that remission encompasses partial and complete remission. Remission can occur for any period of time, such as from one month to several years or more.

“Relapse” refers to the clinical diagnosis of a return of breast cancer after a period of remission.

“Relapse-free survival” or “RFS” refers to the time from the date of diagnosis of breast cancer to the date of relapse.

“Good prognosis” refers to a normal risk of relapse, a reduced risk of relapse, an increased chance for remission, an increased relapse-free survival time, or a high survival rate. In embodiments, a “good prognosis” refers to a reduced risk of relapse, an increased chance for remission, an increased relapse-free survival time, or a high survival rate. In embodiments, a “good prognosis” refers to a reduced risk of relapse. In embodiments, a “good prognosis” refers to an increased chance for remission. In embodiments, a “good prognosis” refers to an increased relapse-free survival time. In embodiments, a “good prognosis” refers to a high survival rate. In embodiments, a high survival rate refers to a 5-year survival rate greater than 50%. In embodiments, a high survival rate refers to a 5-year survival rate greater than 60%, greater than 70%, greater than 80%, or greater than 90%. In embodiments, “good prognosis” is an increased likelihood of a good prognosis.

“Biological sample” refers to a material of biological origin (e.g., blood, plasma, cells, tissues, organs, fluids). In embodiments, biological sample is blood. In embodiments, the biological sample is a tumor. In embodiments, the biological sample is tumor tissue. In embodiments, the biological sample is tumor cells.

“Peripheral blood” refers to blood circulating throughout the body. The components of peripheral blood include red blood cells (erythrocytes), white blood cells (leukocytes), and platelets.

“Peripheral blood mononuclear cell” or “PBMC” refers to cells in peripheral blood that have a nucleus, generally a round nucleus. Exemplary peripheral blood mononuclear cells include lymphocytes and monocytes. Exemplary lymphocytes are T cells, B cells, and NK cells.

“Gene expression” refers to the conversion of genetic information from genes via messenger RNA (mRNA) to proteins. The genetic information (base sequence) on DNA is copied to a molecule of mRNA (transcription). The mRNA molecules then leave the cell nucleus and enter the cytoplasm, where they participate in protein synthesis by specifying the particular amino acids that make up individual proteins (translation).

The terms an “elevated level” or an “increased level” or a “high level” of gene expression is a expression level of the gene or protein that is higher than the expression level of the gene or protein in a standard control. The standard control may be any suitable control, examples of which are described herein.

The terms a “reduced level” or a “decreased expression level” or a “low level” of gene expression is a expression level of the gene or protein that is lower than the expression level of the gene or protein in a standard control. The standard control may be any suitable control, examples of which are described herein.

“Pathway” refers to a set of system components involved in two or more sequential molecular interactions that result in the production of a product or activity. A pathway can produce a variety of products or activities that can include, for example, intermolecular interactions, changes in expression of a nucleic acid or polypeptide, the formation or dissociation of a complex between two or more molecules, accumulation or destruction of a metabolic product, activation or deactivation of an enzyme or binding activity. Thus, the term “pathway” includes a variety of pathway types, such as, for example, a biochemical pathway, a gene expression pathway, and a regulatory pathway. Similarly, a pathway can include a combination of these exemplary pathway types.

“Control,” “standard control,” or “control experiment” is used in accordance with its plain ordinary meaning and refers to an experiment in which the subjects or reagents of the experiment are treated as in a parallel experiment except for omission of a procedure, reagent, or variable of the experiment. In embodiments, the control is used as a standard of comparison in evaluating experimental effects. In embodiments, a control is the measurement of the activity of a protein in the absence of a compound as described herein (including embodiments and examples). For example, a test sample can be taken from a patient suspected of having a given disease (cancer) and compared to samples from a known cancer patient or a known normal (non-disease) individual. A control can also represent an average value gathered from a population of similar individuals, e.g., cancer patients or healthy individuals with a similar medical background, same age, weight, etc. A control can also be obtained from the same individual, e.g., from an earlier-obtained sample, prior to disease, or prior to treatment. One of skill will recognize that controls can be designed for assessment of any number of parameters. In embodiments, a control is a negative control. In embodiments, such as some embodiments relating to detecting the expression level of a gene/protein or a subset of genes/proteins, a control comprises the average amount of expression (e.g., protein or mRNA) in a population of subjects (e.g., with cancer) or in a healthy or general population. In embodiments, the control comprises an average amount (e.g. amount of expression) in a population in which the number of subjects (n) is 10 or more, 25 of more, 50 or more, 100 or more, 1000 or more, or 5000 or more. In embodiments, the control is a population of cancer subjects. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. In embodiments, the standard control is a CD8+ T cell population from a healthy subject, a population of healthy subjects, a breast cancer patient who is responsive to treatment with a non-chemotherapeutic therapy (e.g., hormone therapy), or a population of breast cancer patients who are responsive to treatment with a non-chemotherapeutic therapy (e.g., hormone therapy). In embodiments, the standard control is a CD26+CD4+ T cell population from a healthy subject, a population of healthy subjects, a breast cancer patient who is responsive to treatment with a non-chemotherapeutic therapy (e.g., hormone therapy), or a population of breast cancer patients who are responsive to treatment with a non-chemotherapeutic therapy (e.g., hormone therapy).

The terms “treating” and “treatment” refers to any indicia of success in the therapy or amelioration of an injury, disease, pathology or condition, including any objective or subjective parameter such as abatement; remission; diminishing of symptoms or making the pathology or condition more tolerable to the patient; slowing in the rate of degeneration or decline; or making the final point of degeneration less debilitating. The treatment or amelioration of symptoms can be based on objective or subjective parameters; including the results of a physical examination. Treating does not include preventing.

“Treating” or “treatment” as used herein also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, delay or slowing of disease progression, amelioration or palliation of the disease state, diminishment of the reoccurrence of disease, and remission, whether partial or total and whether detectable or undetectable. Treatment may inhibit the disease's spread; relieve the disease's symptoms, fully or partially remove the disease's underlying cause, shorten a disease's duration, or do a combination of these things.

“Patient” or “subject” in need thereof refers to a living organism suffering from or prone to a disease or condition that can be treated by administration of a pharmaceutical composition as provided herein. In embodiments, a patient is human. In embodiments, a patient is a human with breast cancer. In embodiments, a patient is a woman with breast cancer. In embodiments, a patient is a man with breast cancer. In embodiments, a patient is a premenopausal woman with breast cancer. In embodiments, a patient is a postmenopausal woman with breast cancer.

“Breast cancer patient” or “breast cancer subject” refers to a patient or subject with breast cancer. “Breast cancer patient” can alternatively be referred to as a patient with breast cancer.

“Breast cancer” refers to a malignant tumor that develops from cells in the breast. Usually breast cancer begin: (i) in the cells of the lobules, which are the milk-producing glands; (ii) in the cells of the ducts, the passages that drain milk from the lobules to the nipple; or (iii) in the stromal tissues, which include the fatty and fibrous connective tissues of the breast. Types of breast cancer include ductal carcinoma in situ, invasive ductal carcinoma, tubular carcinoma of the breast, medullary carcinoma of the breast, mucinous carcinoma of the breast, papillary carcinoma of the breast, cribriform carcinoma of the breast, invasive lobular carcinoma, inflammatory breast cancer, lobular carcinoma in situ, and the like. The breast cancer may also be of a molecular sub-type, such as luminal A, luminal B, triple negative, HER2, ER+, PR+, ER+/PR+ and normal-like. The breast cancer can be primary breast cancer or metastatic breast cancer. The breast cancer can be any stage, including Stage 0, IA, IB, IIA, IIB, IIIA, IIIB, IIIC, or IV. In embodiments, the breast cancer is triple negative, ER+, PR+, or ER+/PR+. In embodiments, the breast cancer is ER+, PR+, or ER+/PR+. In embodiments, the breast cancer is ER+.

Methods

The disclosure provides methods of predicting increased risk of relapse or reduced survival in a breast cancer in a patient in need thereof comprising (i) detecting a level of exhausted CD8+ T cells in a biological sample obtained from the patient; (ii) identifying the level of the exhausted CD8+ T cells as being higher than a standard control or as being lower than a standard control; and (iii) predicting the patient to have a good prognosis when the level of the exhausted CD8+ T cells is lower than the standard control and predicting the patient to have an increased risk of relapse or reduced survival when the level of the exhausted CD8+ T cells is greater than the standard control. In embodiments, the breast cancer is estrogen receptor positive (ER+) breast cancer and the patient is a premenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of detecting an elevated level of exhausted CD8+ T cells in patient having breast cancer, the method comprising detecting an elevated level of exhausted CD8+ T cells, relative to a control, in a biological sample obtained from the patient. In embodiments, the breast cancer is estrogen receptor positive (ER+) breast cancer and the patient is a premenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of treating breast cancer in a patient in need thereof comprising (i) detecting a level of exhausted CD8+ T cells in a biological sample obtained from the patient; (ii) identifying the level of the exhausted CD8+ T cells as being higher than a standard control or as being lower than a standard control; and (iii) administering to the patient: (a) an effective amount of a non-chemotherapeutic therapy when the level of the exhausted CD8+ T cells is lower than the standard control, thereby treating the breast cancer in the patient; or (b) an effective amount of a chemotherapeutic therapy when the level of the exhausted CD8+ T cells is greater than the standard control, thereby treating the breast cancer in the patient. In embodiments, the breast cancer is estrogen receptor positive (ER+) breast cancer and the patient is a premenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of identifying a breast cancer patient having increased risk of relapse or reduced survival comprising detecting an elevated level of exhausted CD8+ T cells, relative to a standard control, in a biological sample obtained from the patient, wherein the elevated level of exhausted CD8+ T cells indicates the patient as having increased risk of relapse or reduced survival. In embodiments, the method further comprising administering to the patient an effective amount of a chemotherapeutic therapy. The disclosure provides methods of selecting a patient having breast cancer for treatment with a chemotherapeutic therapy comprising selecting the patient for a chemotherapeutic therapy when the patient has an elevated level of exhausted CD8+ T cells relative to a standard control. In embodiments, the method further comprising administering to the patient an effective amount of a chemotherapeutic therapy. In embodiments, the breast cancer is estrogen receptor positive (ER+) breast cancer and the patient is a premenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of treating breast cancer in a patient in need thereof comprising administering to the patient an effective amount of a chemotherapeutic therapy, wherein a biological sample obtained from the patient has an elevated level of exhausted CD8+ T cells relative to a standard control. The disclosure provides methods of treating breast cancer in a patient in need thereof comprising: (i) detecting an elevated level of exhausted CD8+ T cells, relative to a standard control, in a biological sample obtained from the patient; and (ii) administering to the patient an effective amount of a chemotherapeutic therapy. In embodiments, the breast cancer is estrogen receptor positive (ER+) breast cancer and the patient is a premenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of identifying a breast cancer patient having a good prognosis comprising detecting a level of exhausted CD8+ T cells lower than a standard control, in a biological sample obtained from the patient, wherein the decreased expression level of exhausted CD8+ T cells indicates the patient has a good prognosis. In embodiments, the method further comprising administering to the patient an effective amount of a non-chemotherapeutic therapy. The disclosure provides methods of selecting a patient having breast cancer for treatment with a non-chemotherapeutic therapy comprising selecting the patient for a non-chemotherapeutic therapy when the patient has a level of exhausted CD8+ T cells lower than a standard control. In embodiments, the method further comprising administering to the patient an effective amount of a non-chemotherapeutic therapy. In embodiments, the breast cancer is estrogen receptor positive (ER+) breast cancer and the patient is a premenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of treating breast cancer in a patient in need thereof comprising administering to the patient an effective amount of a non-chemotherapeutic therapy, wherein a biological sample obtained from the patient has a level of exhausted CD8+ T cells lower than a standard control. The disclosure provides methods of treating breast cancer in a patient in need thereof comprising (i) detecting a level of exhausted CD8+ T cells lower than a standard control in a biological sample obtained from the patient; and (ii) administering to the patient an effective amount of a non-chemotherapeutic therapy. In embodiments, the breast cancer is estrogen receptor positive (ER+) breast cancer and the patient is a premenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

In embodiments, the exhausted CD8+ T cells are PD-1′. In embodiments, the exhausted CD8+ T cells are CD39*. In embodiments, the exhausted CD8+ T cells are PD-1+ and CD39+. In embodiments, the exhausted CD8+ T cells express PD-1 (PD-1+) and express CD39 (CD39+). In embodiments, the exhausted CD8+ T cells express PD-1 (PD-1+). In embodiments, the exhausted CD8+ T cells express CD39 (CD39+). In embodiments, the exhausted CD8+ T cells have an increased expression level of PD-1 relative to standard control (PD-1+) and an increased expression level of CD39 relative to standard control (CD39+). In embodiments, the exhausted CD8+ T cells have an increased expression level of PD-1 relative to standard control. In embodiments, the exhausted CD8+ T cells have an increased expression level of CD39 relative to standard control.

In embodiments, the exhausted CD8+ T cells have a decreased expression of TNFα, IL-2, CD127, KLRG1, activator protein 1, JUNB, FOS, or a combination thereof, relative to a standard control. In embodiments, the exhausted CD8+ T cells have a decreased expression of TNFα, IL-2, CD127, KLRG1, activator protein 1, JUNB, FOS, or a combination thereof, relative to a standard control; and an increased expression level of CD137 relative to a standard control. In embodiments, the exhausted CD8+ T cells have a decreased expression of TNFα, IL-2, or a combination thereof, relative to a standard control. In embodiments, the exhausted CD8+ T cells have a decreased expression of TNFα relative to a standard control. In embodiments, the exhausted CD8+ T cells have a decreased expression of IL-2 relative to a standard control. The method of any one of claims 1 to 5, wherein the exhausted CD8+ T cells have a decreased expression of CD127, KLRG1, or a combination thereof, relative to a standard control. The method of any one of claims 1 to 5, wherein the exhausted CD8+ T cells have a decreased expression of CD127 relative to a standard control. The method of any one of claims 1 to 5, wherein the exhausted CD8+ T cells have a decreased expression of KLRG1 relative to a standard control. In embodiments, the exhausted CD8+ T cells have a decreased expression of activator protein 1, JUNB, FOS, or a combination of two or more thereof, relative to a standard control. In embodiments, the exhausted CD8+ T cells have a decreased expression of activator protein 1 relative to a standard control. In embodiments, the exhausted CD8+ T cells have a decreased expression of JUNB relative to a standard control. In embodiments, the exhausted CD8+ T cells have a decreased expression of FOS relative to a standard control. In embodiments, the exhausted CD8+ T cells have an increased expression level of CD137 relative to a standard control.

In embodiments, an increased level of exhausted CD8+ T cells comprises an increased expression level of at least one gene from Table 1 (i.e., at least one gene selected from the group consisting of CXCL13, GZMB, IFI6, HLA-DRA, HLA-DQA2, HLA-DRB5, PRF1, MX1, HLA-DRB1, CD82, LY6E, IFIT3, ISG15, IFI44, IFITM3, ENTPD1, OAS1, IFI44L, BST2, GNLY, HAVCR2, KRT86, ALOX5AP, CCL3, and IFI27) relative to a standard control. This will be understood by the skilled artisan to conversely mean that a decreased expression level of exhausted CD8+ T cells comprises a decreased expression level of at least one gene from Table 1 relative to a standard control. In embodiments, an increased level of exhausted CD8+ T cells comprises an unequally weighted average of the increased expression level of at least two genes from Table 1.

In embodiments, an increased level of exhausted CD8+ T cells comprises an increased expression level of at least 2 genes from Table 1, relative to a control (i.e., there is an increased expression level of at least 2 genes from Table 1). In embodiments, there is an increased expression level of at least 3 genes from Table 1. In embodiments, there is an increased expression level of at least 4 genes from Table 1, relative to a control. In embodiments, there is an increased expression level of at least 5 genes from Table 1, relative to a control. In embodiments, there is an increased expression level of at least 6 genes from Table 1, relative to a control. In embodiments, there is an increased expression level of at least 7 genes from Table 1, relative to a control. In embodiments, there is an increased expression level of at least 8 genes from Table 1, relative to a control. In embodiments, there is an increased expression level of at least 9 genes from Table 1, relative to a control. In embodiments, there is an increased expression level of at least 10 genes from Table 1, relative to a control. In embodiments, there is an increased expression level of at least 11 genes from Table 1, relative to a control. In embodiments, there is an increased expression level of at least 12 genes from Table 1, relative to a control. In embodiments, there is an increased expression level of at least 13 genes from Table 1, relative to a control. In embodiments, there is an increased expression level of at least 14 genes from Table 1, relative to a control. In embodiments, there is an increased expression level of at least 15 genes from Table 1, relative to a control. In embodiments, there is an increased expression level of at least 16 genes from Table 1, relative to a control. In embodiments, there is an increased expression level of at least 17 genes from Table 1, relative to a control. In embodiments, there is an increased expression level of at least 18 genes from Table 1, relative to a control. In embodiments, there is an increased expression level of at least 19 genes from Table 1, relative to a control. In embodiments, there is an increased expression level of at least 20 genes from Table 1, relative to a control. In embodiments, there is an increased expression level of at least 21 genes from Table 1, relative to a control. In embodiments, there is an increased expression level of at least 22 genes from Table 1, relative to a control. In embodiments, there is an increased expression level of at least 23 genes from Table 1, relative to a control. In embodiments, there is an increased expression level of at least 24 genes from Table 1, relative to a control. In embodiments, there is an increased expression level of 2 genes, 3 genes, 4 genes, 5, genes, 6 genes, 7 genes, 8 genes, 9 genes, 10 genes, 11 genes, 12 genes, 13, genes, 14 genes, 15 genes, 16 genes, 17 genes, 18 genes, 19, genes, 20 genes, 21 genes, 22, genes, 23 genes, or 24 genes from Table 1, relative to a control. In embodiments, an increased level of exhausted CD8+ T cells comprises an increased expression level of 25 genes in Table 1, relative to a control. In embodiments, an increased level of exhausted CD8+ T cells comprises an unequally weighted average of the increased expression level of the at least 2-25 genes from Table 1, relative to a control.

TABLE 1
T_{EX} Gene Signature Panel
T_{EX} Gene Signature Panel

| | | | | |
|---|---|---|---|---|
| CXCL13 | HLA-DRB5 | LY6E | ENTPD1 | HAVCR2 |
| GZMB | PRF1 | IFIT3 | OAS1 | KRT86 |
| IFI6 | MX1 | ISG15 | IFI44L | ALOX5AP |
| HLA-DRA | HLA-DRB1 | IFI44 | BST2 | CCL3 |
| HLA-DQA2 | CD82 | IFITM3 | GNLY | IFI27 |

The genes set forth in Table 1 are known in the art and the proteins encoded by the genes set forth in Table 1 are known in the art. The nucleic acid sequence of the genes, the nucleic acid sequence of the RNA (e.g., mRNA) encoded by the genes, and the amino acid sequence of the proteins encoded by the genes are known in the art, and can be found, for example, at the National Library of Medicine, National Center for Biotechnology Information at www.ncbi.nlm.nih.gov, at UniProt at www.uniprot.org, and the like.

The disclosure provides methods of predicting increased risk of relapse or reduced survival in a breast cancer patient comprising (i) detecting a expression level of at least one gene from Table 1 in a biological sample obtained from the patient; (ii) identifying the expression level of the at least one gene from Table 1 as being higher than a standard control or as being less than a standard control; and (iii) predicting a good prognosis when the expression level of the at least one gene from Table 1 is lower than the standard control; and predicting an increased risk of relapse or reduced survival when the expression level of the at least one gene from Table 1 is greater than the standard control. In embodiments, the method further comprises administering to the patient an effective amount of a chemotherapeutic therapy when the patient is at increased risk of relapse or reduced survival. In embodiments, the method further comprises administering to the patient an effective amount of a non-chemotherapeutic therapy when the patient has a good prognosis. The disclosure provides methods of identifying a breast cancer patient for treatment with a chemotherapeutic therapy comprising (i) detecting a expression level of at least one gene from Table 1 in a biological sample obtained from the patient; (ii) identifying the expression level of the at least one gene from Table 1 as being higher than a standard control or as being lower than a standard control; and (iii) identifying the breast cancer patient for treatment with a non-chemotherapeutic therapy when the expression level of the at least one gene from Table 1 is lower than the standard control; and identifying the breast cancer patient for treatment with a chemotherapeutic therapy when the expression level of the at least one gene from Table 1 is greater than the standard control. In embodiments, the method further comprises administering to the patient an effective amount of a chemotherapeutic therapy. In embodiments, the breast cancer is estrogen receptor positive (ER+) breast cancer and the patient is a premenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of treating breast cancer in a patient in need thereof comprising (i) detecting a expression level of at least one gene from Table 1 in a biological sample obtained from the patient; (ii) identifying the expression level of the at least one gene from Table 1 as higher than a standard control or as lower than a standard control; and (iii) administering to the patient: (a) an effective amount of a non-chemotherapeutic therapy when the expression level of the at least one gene from Table 1 is lower than the standard control, thereby treating the breast cancer in the patient; or (b) an effective amount of a chemotherapeutic therapy when the expression level of the at least one gene from Table 1 is greater than the standard control, thereby treating the breast cancer in the patient. In embodiments, the breast cancer is estrogen receptor positive (ER+) breast cancer and the patient is a premenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of monitoring the progression of breast cancer in a patient or the efficacy of a treatment in a breast cancer patient comprising (i) detecting a expression level of at least one gene from Table 1 in a first biological sample obtained from the patient, (i) detecting a expression level of at least one gene from Table 1 in a second biological sample obtained from the patient, wherein the second biological sample is obtained at a time later than the first biological sample; and (iii) determining that breast cancer patient has an improvement or that the treatment is efficacious when the expression level of the at least one gene from Table 1 in the second biological sample is lower than the expression level of the at least one gene from Table 1 in the first biological sample. In embodiments, the treatment is a chemotherapeutic therapy. In embodiments, the treatment is a non-chemotherapeutic therapy. In embodiments, the breast cancer is estrogen receptor positive (ER+) breast cancer and the patient is a premenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of identifying a patient having breast cancer as having increased risk of relapse or reduced survival comprising identifying the patient as having increased risk of relapse or reduced survival when the patient has an elevated expression level at least one gene selected from Table 1 relative to a standard control. In embodiments, the method further comprises administering to the patient an effective amount of a chemotherapeutic therapy. The disclosure provides methods of selecting a patient having breast cancer for treatment with a chemotherapeutic therapy comprising selecting the patient for treatment with a chemotherapeutic therapy when the patient has an elevated expression level of at least one gene from Table 1 relative to a standard control. In embodiments, the method further comprises administering to the patient an effective amount of a chemotherapeutic therapy. In embodiments, the elevated expression level of the at least one gene from Table 1 comprises an unequally weighted average of the elevated expression level of the at least one gene from Table 1. In embodiments, the breast cancer is estrogen receptor positive (ER+) breast cancer and the patient is a premenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of treating breast cancer in a patient in need thereof comprising administering to the patient an effective amount of a chemotherapeutic therapy, wherein the patient has an elevated expression level of at least one gene from Table 1 (i.e., at least one gene selected from the group consisting of CXCL13, GZMB, IFI6, HLA-DRA, HLA-DQA2, HLA-DRB5, PRF1, MX1, HLA-DRB1, CD82, LY6E, IFIT3, ISG15, IFI44, IFITM3, ENTPD1, OAS1, IFI44L, BST2, GNLY, HAVCR2, KRT86, ALOX5AP, CCL3, and IFI27) relative to a standard control. The disclosure provides methods of treating breast cancer in a patient in need thereof comprising: (i) detecting an elevated expression level of at least one gene from Table 1 relative to a standard control in a biological sample obtained from the patient; and (ii) administering to the patient an effective amount of a chemotherapeutic therapy. In embodiments, the elevated expression level of the at least one gene from Table 1 comprises an unequally weighted average of the elevated expression level of the at least one gene from Table 1. In embodiments, the breast cancer is estrogen receptor positive (ER+) breast cancer and the patient is a premenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

In embodiments, the patient has an increased expression level of at least 2 genes from Table 1, relative to a control. In embodiments, the patient has an increased expression level of at least 3 genes from Table 1, relative to a control. In embodiments, the patient has an increased expression level of at least 4 genes from Table 1, relative to a control. In embodiments, the patient has an increased expression level of at least 5 genes from Table 1, relative to a control. In embodiments, the patient has an increased expression level of at least 6 genes from Table 1, relative to a control. In embodiments, the patient has an increased expression level of at least 7 genes from Table 1, relative to a control. In embodiments, the patient has an increased expression level of at least 8 genes from Table 1, relative to a control. In embodiments, the patient has an increased expression level of at least 9 genes from Table 1, relative to a control. In embodiments, the patient has an increased expression level of at least 10 genes from Table 1, relative to a control. In embodiments, the patient has an increased expression level of at least 11 genes from Table 1, relative to a control. In embodiments, the patient has an increased expression level of at least 12 genes from Table 1, relative to a control. In embodiments, the patient has an increased expression level of at least 13 genes from Table 1, relative to a control. In embodiments, the patient has an increased expression level of at least 14 genes from Table 1, relative to a control. In embodiments, the patient has an increased expression level of at least 15 genes from Table 1, relative to a control. In embodiments, the patient has an increased expression level of at least 16 genes from Table 1, relative to a control. In embodiments, the patient has an increased expression level of at least 17 genes from Table 1, relative to a control. In embodiments, the patient has an increased expression level of at least 18 genes from Table 1, relative to a control. In embodiments, the patient has an increased expression level of at least 19 genes from Table 1, relative to a control. In embodiments, the patient has an increased expression level of at least 20 genes from Table 1, relative to a control. In embodiments, the patient has an increased expression level of at least 21 genes from Table 1, relative to a control. In embodiments, the patient has an increased expression level of at least 22 genes from Table 1, relative to a control. In embodiments, the patient has an increased expression level of at least 23 genes from Table 1, relative to a control. In embodiments, the patient has an increased expression level of at least 24 genes from Table 1, relative to a control. In embodiments, the patient has an increased expression level of 2 genes, 3 genes, 4 genes, 5, genes, 6 genes, 7 genes, 8 genes, 9 genes, 10 genes, 11 genes, 12 genes, 13, genes, 14 genes, 15 genes, 16 genes, 17 genes, 18 genes, 19, genes, 20 genes, 21 genes, 22, genes, 23 genes, or 24 genes from Table 1, relative to a control. In embodiments, the patient has an increased expression level of 25 genes in Table 1, relative to a control. In embodiments, the increased expression level of the at least 2-25 genes from Table 1 comprises an unequally weighted average of the increased expression level of the at least 2-25 genes from Table 1.

In embodiments, the methods further comprise determining the patient's breast cancer risk score based on the Oncotype Dx® test or an equivalent thereof. Oncotype Dx® is based on a panel of 21 genes including proliferation-related genes (Ki67, STK15, BIRC5, CCNB1, MYBL2), metastasis-related genes (MMP11, CTSL2), HER2-related genes (GRB7, HER2), sex hormone-related genes (ER, PGR, BCL2, SCUBE2, GSTM1, BAG1, CD68), and internal control genes (ACTB, GAPDH, GUS, RPLPO and TFRC). Oncotype Dx® is an assay that detects the expression level of the genes, applies an algorithm to the unequally weighted expression levels of the genes, and produces a Breast Recurrence Score®. The Breast Recurrence Score® predicts the chances of a patient having increased risk of relapse or reduced survival. A low risk scores is 0-10, an intermediate risk score is 11-25, and a high risk score is 26-100. When a patient has a low risk score, they are selected for treatment with non-chemotherapeutic therapy. When a patient has a high risk score, they are selected for treatment with chemotherapeutic therapy. An issue arises when a patient has an intermediate risk score because it is unclear if the patient does or does not have an increase risk of relapse and whether the patient should be treated with chemotherapeutic therapy, which has more debilitating side-effects than other types of treatment. There is a need in the art to identify the best treatment for breast cancer patients having an intermediate risk score in the Oncotype Dx® test or an equivalent thereof. See Crolley et al, Breast Cancer Res Treat, 180(3):809-817 (2020).

The inventors have discovered that the challenges associated with the intermediate Breast Recurrence Score® in the Oncotype Dx® test (or an equivalent thereof) can be resolved by further determining if the breast cancer patient having an intermediate Breast Recurrence Score® has exhausted CD8⁺ T cells, as described herein or has an elevated expression level of at least one gene from Table 1 (e.g., an unequally weighted average of the elevated gene expression levels of the genes in Table 1). If the patient having an intermediate Breast Recurrence Score® is identified as having increased risk of relapse or reduced survival based on the level of exhausted CD8⁺ T cells or based on the gene expression levels of the genes in Table 1, the patient is considered to have increased risk of relapse or reduced survival and is then administered a chemotherapeutic therapy.

Thus, in embodiments, the methods further comprise identifying a patient as having an intermediate breast cancer risk score based on the Oncotype Dx® test (or an equivalent thereof) prior to detecting an elevated expression level of at least one gene from Table 1. In embodiments, the methods further comprise identifying a patient as having an intermediate breast cancer risk score when a biological sample obtained from the patient has an intermediate gene expression level of at least one gene selected from the group consisting of K67, STK15, BIRC5, CCNB1, MYBL2, MMP11, CTSL2, GRB7, HER2, ER, PGR, BCL2, SCUBE2, GSTM1, BAG1, and CD68, relative to a standard control. In embodiments, the methods further comprises identifying a patient as having an intermediate breast cancer risk score when a biological sample obtained from the patient has an intermediate gene expression level of K167, STK15, BIRC5, CCNB1, MYBL2, MMP11, CTSL2, GRB7, HER2, ER, PGR, BCL2, SCUBE2, GSTM1, BAG1, and CD68, relative to a standard control. In embodiments, the methods further comprises identifying a patient as having an intermediate breast cancer risk score when the intermediate gene expression levels are based on an equally weighted average of the intermediate gene expression levels of Ki67, STK15, BIRC5, CCNB1, MYBL2, MMP11, CTSL2, GRB7, HER2, ER, PGR, BCL2, SCUBE2, GSTM11, BAG1, and CD68, relative to a standard control. In embodiments, the methods further comprise identifying an intermediate gene expression levels of Ki67, STK15, BIRC5, CCNB1, MYBL2, MMP11, CTSL2, GRB7, HER2, ER, PGR, BCL2, SCUBE2, GSTM1, BAG1, and CD68, relative to a standard control, a biological sample obtained from the patient.

In embodiments, the methods further comprise identifying a patient as having an intermediate breast cancer risk score when a biological sample obtained from the patient has an intermediate gene expression level of at least one gene selected from the group consisting of Ki67, STK15, BIRC5, CCNB1, MYBL2, MMP11, CTSL2, GRB7, HER2, ER, PGR, BCL2, SCUBE2, GSTM1, BAG1, CD68, ACTB, GAPDH, GUS, RPLPO and TFRC relative to a standard control. In embodiments, the methods further comprises identifying a patient as having an intermediate breast cancer risk score when a biological sample obtained from the patient has an intermediate gene expression level of Ki67, STK115, BIRC5, CCNB1, MYBL2, MMP11, CTSL2, GRB7, HER2, ER, PGR, BCL2, SCUBE2, GSTM1, BAG1, CD68, ACTB, GAPDH, GUS, RPLPO and TFRC relative to a standard control. In embodiments, the methods further comprises identifying a patient as having an intermediate breast cancer risk score when the intermediate gene expression levels are based on an equally weighted average of the intermediate gene expression levels of Ki67, STK15, BIRC5, CCNB1, MYBL2, MMP11, CTSL2, GRB7, HER2, ER, PGR, BCL2, SCUBE2, GSTM1, BAG1, CD68, ACTB, GAPDH, GUS, RPLPO and TFRC relative to a standard control. In embodiments, the methods further comprise identifying an intermediate gene expression levels of Ki67, STK15, BIRC5, CCNB1, MYBL2, MMP11, CTSL2, GRB7, HER2, ER, PGR, BCL2, SCUBE2, GSTM1, BAG1, CD68, ACTB, GAPDH, GUS. RPLPO and TFRC relative to a standard control, a biological sample obtained from the patient.

Post-menopausal estrogen receptor positive breast cancer patients with intermediate or low scores on the Oncotype Dx® test (or an equivalent thereof) are considered at low risk for relapse and generally not given chemotherapy. Nonetheless, the methods described herein can be applied to these patients to select out a higher risk group within this patient population for further treatment and/or monitoring. Thus, in embodiments, the methods further comprise identifying a patient as having an intermediate or low breast cancer risk score based on the Oncotype Dx® test (or an equivalent thereof) prior to detecting an elevated expression level of at least one gene from Table 1. In embodiments, the methods further comprise identifying a patient as having an intermediate or low breast cancer risk score when a biological sample obtained from the patient has an intermediate or low gene expression level of at least one gene selected from the group consisting of Ki67, STK15, BIRC5, CCNB1, MYBL2, MMP11, CTSL2, GRB7, HER2, ER, PGR, BCL2, SCUBE2, GSTM1, BAG1, and CD68, relative to a standard control. In embodiments, the methods further comprises identifying a patient as having an intermediate or low breast cancer risk score when a biological sample obtained from the patient has an intermediate or low gene expression level of Ki67, STK15, BIRC5, CCNB1, MYBL2, MMP11, CTSL2, GRB37, HER2, ER, PGR, BCL2, SCUBE2, GSTM1, BAG1, and CD68, relative to a standard control. In embodiments, the methods further comprises identifying a patient as having an intermediate or low breast cancer risk score when the intermediate or low gene expression levels are based on an equally weighted average of the intermediate gene expression levels of Ki67, STK15, BIRC5, CCNB1, MYBL2, MMP11, CTSL2, GRB7, HER2, ER, PGR, BCL2, SCUBE2, GSTM1, BAG1, and CD68, relative to a standard control. In embodiments, the methods further comprise identifying intermediate or low gene expression levels of Ki67, STK15, BIRC5, CCNB1, MYBL2, MMP1, CTSL2, GRB7, HER2, ER, PGR, BCL2, SCUBE2, GSTM1, BAG1, and CD68, relative to a standard control, a biological sample obtained from the patient. In embodiments, the methods further comprise identifying a patient as having an intermediate or low breast cancer risk score when a biological sample obtained from the patient has an intermediate or low gene expression level of at least one gene selected from the group consisting of Ki67, STK15, BIRC5, CCNB1, MYBL2, MMP11, CTSL2, GRB7, HER2, ER, PGR, BCL2, SCUBE2, GSTM1, BAG1, CD68, ACTB, GAPDH, GUS, RPLPO and TFRC relative to a standard control. In embodiments, the methods further comprises identifying a patient as having an intermediate or low breast cancer risk score when a biological sample obtained from the patient has an intermediate or low gene expression level of Ki67, STK15, BIRC5, CCNB1, MYBL2, MMP11, CTSL2, GRB7, HER2, ER, PGR, BCL2, SCUBE2, GSTM1, BAG1, CD68, ACTB, GAPDH, GUS, RPLPO and TFRC relative to a standard control. In embodiments, the methods further comprises identifying a patient as having an intermediate or low breast cancer risk score when the intermediate or low gene expression levels are based on an equally weighted average of the intermediate gene expression levels of Ki67, STK15, BIRC5, CCNB1, MYBL2, MMP1, CTSL2, GRB7, HER2, ER, PGR, BCL2, SCUBE2, GSTM1, BAG1, and CD68, relative to a standard control. In embodiments, the methods further comprise identifying intermediate or low gene expression levels of Ki67, STK15, BIRC5, CCNB1, MYBL2, MMP11, CTSL2, GRB7, HER2, ER, PGR, BCL2, SCUBE2, GSTM1, BAG1, CD68, ACTB, GAPDH, GUS, RPLPO and TFRC relative to a standard control, a biological sample obtained from the patient.

In embodiments, the methods further comprise identifying a patient as having a low breast cancer risk score based on the Oncotype Dx® test (or an equivalent thereof) prior to detecting an elevated expression level of at least one gene from Table 1. In embodiments, the methods further comprise identifying a patient as having a low breast cancer risk score when a biological sample obtained from the patient has a low gene expression level of at least one gene selected from the group consisting of Ki67, STK15, BIRC5, CCNB1, MYBL2, MMP11, CTSL2, GRB7, HER2, R, PGR, BCL2, SCUBE2, GSTM1, BAG1, and CD68, relative to a standard control. In embodiments, the methods further comprises identifying a patient as having a low breast cancer risk score when a biological sample obtained from the patient has a low gene expression level of Ki67, STK15, BIRC5, CCNB1, MYBL2, MMP11, CTSL2, GRB7, HER2, ER, PGR, BCL2, SCUBE2, GSTM1, BAG1, and CD68, relative to a standard control. In embodiments, the methods further comprises identifying a patient as having a low breast cancer risk score when the low gene expression levels are based on an equally weighted average of the low gene expression levels of Ki67, STK1 5, BIRC5, CCNB1, MYBL2, MMP11, CTSL2, GRB7, HER2, ER, PGR, BCL2, SCUBE2, GSTM1, BAG1, and CD68, relative to a standard control. In embodiments, the methods further comprise identifying a low gene expression levels of Ki67, STK15, BIRC5, CCNB1, MYBL2, MMP11, CTSL2, GRB7, HER2, ER, PGR, BCL2, SCUBE2, GST M1, BAG1, and CD68, relative to a standard control, a biological sample obtained from the patient. In embodiments, the methods further comprise identifying a patient as having a low breast cancer risk score when a biological sample obtained from the patient has a low gene expression level of at least one gene selected from the group consisting of Ki67, STK15, BIRC5, CCNB1, MYBL2, MMP11, CTSL2, GRB7, HER2, ER, PGR, BCL2, SCUBE2, GSTM1, BAG1, CD68, ACTB, GAPDH, GUS, RPLPO and TFRC relative to a standard control. In embodiments, the methods further comprises identifying a patient as having a low breast cancer risk score when a biological sample obtained from the patient has a low gene expression level of Ki67, STK15, BIRC5, CCNB11, MYBL2, MMP11, CTSL2, GRB7, HER2, ER, PGR, BCL2, SCUBE2, GSTM1, BAG1, CD68, ACTB, GAPDH, GUS, RPLPO and TFRC relative to a standard control. In embodiments, the methods further comprises identifying a patient as having a low breast cancer risk score when the low gene expression levels are based on an equally weighted average of the low gene expression levels of Ki67, STK15, BIRC5, CCNB1, MYBL2, MMP11, CTSL2, GRB7, HER2, ER, PGR, BCL2, SCUBE2, GSTM1, BAG1, CD68, ACTB, GAPDH, GUS, RPLPO and TFRC relative to a standard control. In embodiments, the methods further comprise identifying a low gene expression levels of Ki67, STK15, BIRC5, CCNB1, MYBL2, MMP11, CTSL2, GRB7, HER2, ER, PGR, BCL2, SCUBE2, GSTM1, BAG1, CD68, ACTB, GAPDH, GUS, RPLPO and TFRC relative to a standard control, a biological sample obtained from the patient.

In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments of the methods, the breast cancer is ER+ breast cancer and the patient is a premenopausal woman. In embodiments of the methods, the breast cancer is ER+ breast cancer and the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of identifying a breast cancer patient at increased risk for relapse or reduced survival comprising (i) detecting a level of CD26+CD4+ T cells in a biological sample obtained from the patient; (ii) identifying the level of the CD26+CD4+ T cells as being higher than a standard control or as being lower than a standard control; and (iii) identify the patient as having: (a) a good prognosis when the level of the CD26+CD4+ T cells is greater than the standard control or (b) an increased risk of relapse or reduced survival when the level of the CD26+CD4+ T cells is less than the standard control, thereby treating the breast cancer in the patient. The disclosure provides methods of treating breast cancer in a patient in need thereof comprising (i) detecting a level of CD26+CD4+ T cells in a biological sample obtained from the patient; (ii) identifying the level of the CD26+CD4+ T cells as being higher than a standard control or as being less than a standard control; and (iii) administering to the patient: (a) an effective amount of a non-chemotherapeutic therapy when the level of the CD26+CD4+ T cells is greater than the standard control, thereby treating the breast cancer in the patient; or (b) an effective amount of a chemotherapeutic therapy when the level of the CD26+CD4+ T cells is less than the standard control, thereby treating the breast cancer in the patient. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is triple negative breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of detecting an increased level of CD26+CD4+ T cells in a patient having breast cancer, the method comprising detecting an increased level of CD26+CD4+ T cells, relative to a control, in a biological sample obtained from the patient. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is triple negative breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of treating breast cancer in a patient in need thereof comprising administering to the patient an effective amount of a chemotherapeutic therapy, wherein a biological sample obtained from the patient has a decreased expression level of CD26+CD4+ T cells relative to a standard control. The disclosure provides methods of treating breast cancer in a patient in need thereof comprising: (i) detecting a decreased expression level of CD26+CD4+ T cells, relative to a standard control, in a biological sample obtained from the patient; and (ii) administering to the patient an effective amount of a chemotherapeutic therapy. The disclosure provides methods of identifying a breast cancer patient having increased risk of relapse or reduced survival comprising detecting a decreased expression level of CD26+CD4+ T cells, relative to a standard control, in a biological sample obtained from the patient, wherein the decreased expression level of CD26+CD4+ T cells indicates the patient as having increased risk of relapse or reduced survival. In embodiments, the method further comprising administering to the patient an effective amount of a chemotherapeutic therapy. The disclosure provides methods of selecting a patient having breast cancer for treatment with a chemotherapeutic therapy comprising selecting the patient for treatment with a chemotherapeutic therapy when the patient has a decreased expression level of CD26+CD4+ T cells relative to a standard control. In embodiments, the method further comprising administering to the patient an effective amount of a chemotherapeutic therapy. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is triple negative breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of identifying a breast cancer patient having a good prognosis comprising detecting an increased level of CD26+CD4+ T cells, relative to a standard control, in a biological sample obtained from the patient, wherein the increased level of CD26+CD4+ T cells indicates the patient has a good prognosis. In embodiments, the method further comprising administering to the patient an effective amount of a non-chemotherapeutic therapy. The disclosure provides methods of selecting a patient having breast cancer for treatment with a non-chemotherapeutic therapy comprising selecting the patient for treatment with a non-chemotherapeutic therapy when the patient has an increased level of CD26+CD4+ T cells relative to standard control. In embodiments, the method further comprising administering to the patient an effective amount of a non-chemotherapeutic therapy. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is triple negative breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of treating breast cancer in a patient in need thereof comprising administering to the patient an effective amount of a non-chemotherapeutic therapy, wherein a biological sample obtained from the patient has an increased level of CD26+CD4+ T cells relative to standard control. The disclosure provides methods of treating breast cancer in a patient in need thereof comprising (i) detecting an increased level of CD26+CD4+ T cells relative to standard control in a biological sample obtained from the patient; and (ii) administering to the patient an effective amount of a non-chemotherapeutic therapy. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is triple negative breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

In embodiments, an increased level of CD26+CD4+ T cells comprises an increased expression level of at least one gene from Table 2 (i.e., at least one gene selected from the group consisting of IL7R, KLF2, KLRB1, ANXA1, CCR7, GPR183, NFKBIA, LDHB, FOS, TXNIP, RPS8, RPL32, RPS12, RPS6, RPL34, TPT1, RPS3A, TNFAIP3, JUNB, FTH1, RORA, CD69, SERINC5, RPL13, EEF1A1, BIRC3, RPS4X, CD40LG, RPS18, RPLPO, YPEL5, NAP1L1, SELL, KLF6, RPSA, FOSB, JUN, and RNASET2) relative to a standard control. This will be understood by the skilled artisan to conversely mean that a decreased expression level of CD26+CD4+ T cells comprises a decreased expression level of at least one gene from Table 2 (i.e., at least one gene selected from the group consisting of IL7R, KLF2, KLRB1, ANXA1, CCR7, GPR183, NFKBIA, LDHB, FOS, TXNIP, RPS8, RPL32, RPS12, RPS6, RPL34, TPT1, RPS3A, TNFAIP3, JUNB, FTH1, RORA, CD69, SERINC5, RPL13, EEF1A1, BIRC3, RPS4X, CD40LG, RPS18, RPLPO, YPEL5, NAP1L1, SELL, KLF6, RPSA, FOSB, JUN, and RNASET2) relative to a standard control. In embodiments, an increased level of CD26+CD4+ T cells comprises an unequally weighted average of the increased expression level of at least one gene from Table 2. In embodiments, a decreased level of CD26+CD4+ T cells comprises an unequally weighted average of the decreased expression level of at least one gene from Table 2.

In embodiments, an increased level of CD26+CD4+ T cells comprises an increased expression level of at least 2 genes from Table 2, relative to a control. In embodiments, an increased level of CD26+CD4+ T cells comprises an increased expression level of at least 3 genes from Table 2, relative to a control (i.e., there is an increased expression level of at least 3 genes from Table 2, relative to a control). In embodiments, there is an increased expression level of at least 4 genes from Table 2, relative to a control. In embodiments, there is an increased expression level of at least 5 genes from Table 2, relative to a control. In embodiments, there is an increased expression level of at least 6 genes from Table 2, relative to a control. In embodiments, there is an increased expression level of at least 7 genes from Table 2, relative to a control. In embodiments, there is an increased expression level of at least 8 genes from Table 2, relative to a control. In embodiments, there is an increased expression level of at least 9 genes from Table 2, relative to a control. In embodiments, there is an increased expression level of at least 10 genes from Table 2, relative to a control. In embodiments, there is an increased expression level of at least 11 genes from Table 2, relative to a control. In embodiments, there is an increased expression level of at least 12 genes from Table 2, relative to a control. In embodiments, there is an increased expression level of at least 13 genes from Table 2, relative to a control. In embodiments, there is an increased expression level of at least 14 genes from Table 2, relative to a control. In embodiments, there is an increased expression level of at least 15 genes from Table 2, relative to a control. In embodiments, there is an increased expression level of at least 16 genes from Table 2, relative to a control. In embodiments, there is an increased expression level of at least 17 genes from Table 2, relative to a control. In embodiments, there is an increased expression level of at least 18 genes from Table 2, relative to a control. In embodiments, there is an increased expression level of at least 19 genes from Table 2, relative to a control. In embodiments, there is an increased expression level of at least 20 genes from Table 2, relative to a control. In embodiments, there is an increased expression level of at least 21 genes from Table 2, relative to a control. In embodiments, there is an increased expression level of at least 22 genes from Table 2, relative to a control. In embodiments, there is an increased expression level of at least 23 genes from Table 2, relative to a control. In embodiments, there is an increased expression level of at least 24 genes from Table 2, relative to a control. In embodiments, there is an increased expression level of at least 25 genes in Table 2, relative to a control. In embodiments, there is an increased expression level of at least 26 genes in Table 2, relative to a control. In embodiments, there is an increased expression level of at least 27 genes in Table 2, relative to a control. In embodiments, there is an increased expression level of at least 28 genes in Table 2, relative to a control. In embodiments, there is an increased expression level of at least 29 genes in Table 2, relative to a control. In embodiments, there is an increased expression level of at least 30 genes in Table 2, relative to a control. In embodiments, there is an increased expression level of at least 31 genes in Table 2, relative to a control. In embodiments, there is an increased expression level of at least 32 genes in Table 2, relative to a control. In embodiments, there is an increased expression level of at least 33 genes in Table 2, relative to a control. In embodiments, there is an increased expression level of at least 34 genes in Table 2, relative to a control. In embodiments, there is an increased expression level of at least 35 genes in Table 2, relative to a control. In embodiments, there is an increased expression level of at least 36 genes in Table 2, relative to a control. In embodiments, there is an increased expression level of at least 37 genes in Table 2, relative to a control. In embodiments, an increased level of CD26+CD4+ T cells comprises an increased expression level of 2 genes, 3 genes, 4 genes, 5, genes, 6 genes, 7 genes, 8 genes, 9 genes, 10 genes, 11 genes, 12 genes, 13, genes, 14 genes, 15 genes, 16 genes, 17 genes, 18 genes, 19, genes, 20 genes, 21 genes, 22, genes, 23 genes, or 24 genes, 25 genes, 26 genes, 27 genes, 28 genes, 29 genes, 30 genes, 31 genes, 32 genes, 33 genes, 34 genes, 35 genes, 36 genes, 37 genes, or 38 genes in Table 2, relative to a control. In embodiments, an increased level of CD26+CD4+ T cells comprises an increased expression level of 38 genes in Table 2, relative to a control. In embodiments, the increased expression level of the at least 2-38 genes from Table 2 comprises an unequally weighted average of the increased expression level of the at least 2-38 genes from Table 2.

In embodiments, a decreased expression level of CD26+CD4+ T cells comprises a decreased expression level of at least 2 genes from Table 2, relative to a control. In embodiments, a decreased expression level of CD26+CD4+ T cells comprises a decreased expression level of at least 3 genes from Table 2, relative to a control (i.e., there is a decreased expression level of at least 3 genes from Table 2, relative to a control). In embodiments, there is an decreased expression level of at least 4 genes from Table 2, relative to a control. In embodiments, there is an decreased expression level of at least 5 genes from Table 2, relative to a control. In embodiments, there is an decreased expression level of at least 6 genes from Table 2, relative to a control. In embodiments, there is an decreased expression level of at least 7 genes from Table 2, relative to a control. In embodiments, there is an decreased expression level of at least 8 genes from Table 2, relative to a control. In embodiments, there is an decreased expression level of at least 9 genes from Table 2, relative to a control. In embodiments, there is an decreased expression level of at least 10 genes from Table 2, relative to a control. In embodiments, there is an decreased expression level of at least 11 genes from Table 2, relative to a control. In embodiments, there is an decreased expression level of at least 12 genes from Table 2, relative to a control. In embodiments, there is an decreased expression level of at least 13 genes from Table 2, relative to a control. In embodiments, there is an decreased expression level of at least 14 genes from Table 2, relative to a control. In embodiments, there is an decreased expression level of at least 15 genes from Table 2, relative to a control. In embodiments, there is an decreased expression level of at least 16 genes from Table 2, relative to a control. In embodiments, there is an decreased expression level of at least 17 genes from Table 2, relative to a control. In embodiments, there is an decreased expression level of at least 18 genes from Table 2, relative to a control. In embodiments, there is an decreased expression level of at least 19 genes from Table 2, relative to a control. In embodiments, there is an decreased expression level of at least 20 genes from Table 2, relative to a control. In embodiments, there is an decreased expression level of at least 21 genes from Table 2, relative to a control. In embodiments, there is an decreased expression level of at least 22 genes from Table 2, relative to a control. In embodiments, there is an decreased expression level of at least 23 genes from Table 2, relative to a control. In embodiments, there is an decreased expression level of at least 24 genes from Table 2, relative to a control. In embodiments, there is an decreased expression level of at least 25 genes in Table 2, relative to a control. In embodiments, there is an decreased expression level of at least 26 genes in Table 2, relative to a control. In embodiments, there is an decreased expression level of at least 27 genes in Table 2, relative to a control. In embodiments, there is an decreased expression level of at least 28 genes in Table 2, relative to a control. In embodiments, there is an decreased expression level of at least 29 genes in Table 2, relative to a control. In embodiments, there is an decreased expression level of at least 30 genes in Table 2, relative to a control. In embodiments, there is an decreased expression level of at least 31 genes in Table 2, relative to a control. In embodiments, there is an decreased expression level of at least 32 genes in Table 2, relative to a control. In embodiments, there is an decreased expression level of at least 33 genes in Table 2, relative to a control. In embodiments, there is an decreased expression level of at least 34 genes in Table 2, relative to a control. In embodiments, there is an decreased expression level of at least 35 genes in Table 2, relative to a control. In embodiments, there is an decreased expression level of at least 36 genes in Table 2, relative to a control. In embodiments, there is an decreased expression level of at least 37 genes in Table 2, relative to a control. In embodiments, a decreased expression level of CD26+CD4+ T cells comprises a decreased expression level of 2 genes, 3 genes, 4 genes, 5, genes, 6 genes, 7 genes, 8 genes, 9 genes, 10 genes, 11 genes, 12 genes, 13, genes, 14 genes, 15 genes, 16 genes, 17 genes, 18 genes, 19, genes, 20 genes, 21 genes, 22, genes, 23 genes, or 24 genes, 25 genes, 26 genes, 27 genes, 28 genes, 29 genes, 30 genes, 31 genes, 32 genes, 33 genes, 34 genes, 35 genes, 36 genes, or 37 genes, in Table 2, relative to a control. In embodiments, there is an decreased expression level of 38 genes in Table 2, relative to a control. In embodiments, the decreased expression level of the at least 2-38 genes from Table 2 comprises an unequally weighted average of the decreased expression level of the at least 2-38 genes from Table 2.

TABLE 2
CD26+CD4+ T cell Gene Signature Panel
CD26+CD4+ T Cell Genes

| | | | | |
|---|---|---|---|---|
| IL7R | FOS | RPS3A | EEF1A1 | SELL |
| KLF2 | TXNIP | TNFAIP3 | BIRC3 | KLF6 |
| KLRB1 | RPS8 | JUNB | RPS4X | RPSA |
| ANXA1 | RPL32 | FTH1 | CD40LG | FOSB |
| CCR7 | RPS12 | RORA | RPS18 | JUN |
| GPR183 | RPS6 | CD69 | RPLP0 | RNASET2 |
| NFKBIA | RPL34 | SERINC5 | YPEL5 | — |
| LDHB | TPT1 | RPL13 | NAP1L1 | |

The genes set forth in Table 2 are known in the art and the proteins encoded by the genes set forth in Table 2 are known in the art. The nucleic acid sequence of the genes, the nucleic acid sequence of the RNA (e.g., mRNA) encoded by the genes, and the amino acid sequence of the proteins encoded by the genes are known in the art, and can be found, for example, at the National Library of Medicine, National Center for Biotechnology Information at www.ncbi.nlm.nih.gov, at UniProt at www.uniprot.org, and the like.

The disclosure provides methods of predicting an increased risk of relapse or reduced survival in a breast cancer patient comprising (i) detecting the expression level of at least one gene from Table 2 in a biological sample obtained from the patient; (ii) identifying the expression level of the at least one gene from Table 2 as higher than a standard control or as lower than a standard control; and (iii) predicting a good prognosis when the expression level of the at least one gene from Table 2 is greater than the standard control; and predicting increased risk of relapse or reduced survival when the expression level of the at least one gene from Table 2 is less than the standard control. In embodiments, the method further comprises administering to the patient an effective amount of a chemotherapeutic therapy when the patient is at increased risk or relapse or reduced survival and administering to the patient an effective amount of a non-chemotherapeutic therapy when the patient has a good prognosis. The disclosure provides methods of identifying a breast cancer patient for treatment with a chemotherapeutic therapy or a non-chemotherapeutic therapy comprising (i) detecting a expression level of at least one gene from Table 1 in a biological sample obtained from the patient; (ii) identifying the expression level of the at least one gene from Table 2 as higher than a standard control or as lower than a standard control; and (iii) identifying the breast cancer patient for treatment with a non-chemotherapeutic therapy when the expression level of the at least one gene from Table 2 is higher than the standard control; and identifying the breast cancer patient for treatment with a chemotherapeutic therapy when the expression level of the at least one gene from Table 2 is lower than the standard control. In embodiments, the method further comprises administering to the patient an effective amount of a chemotherapeutic therapy when the expression level of the at least one gene from Table 2 is lower than the standard control and administering to the patient an effective amount of a non-chemotherapeutic therapy when the expression level of the at least one gene from Table 2 is higher than the standard control. The disclosure provides methods of monitoring the progression of breast cancer or the efficacy of a treatment in a breast cancer patient comprising (i) detecting a expression level of at least one gene from Table 2 in a first biological sample obtained from the patient, (i) detecting a expression level of at least one gene from Table 2 in a second biological sample obtained from the patient, wherein the second biological sample is obtained at a time later than the first biological sample; and (iii) determining that breast cancer patient has an improvement or that the treatment is efficacious when the expression level of the at least one gene from Table 2 in the second biological sample is higher than the expression level of the at least one gene from Table 1 in the first biological sample. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is triple negative breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of treating breast cancer in a patient in need thereof comprising (i) detecting a expression level of at least one gene from Table 2 in a biological sample obtained from the patient; (ii) identifying the expression level of the at least one gene from Table 2 as higher than a standard control or as lower than a standard control; and (iii) administering to the patient: (a) an effective amount of a non-chemotherapeutic therapy when the expression level of the at least one gene from Table 2 is higher than the standard control, thereby treating the breast cancer in the patient; or (b) an effective amount of a chemotherapeutic therapy when the expression level of the at least one gene from Table 2 is lower than the standard control, thereby treating the breast cancer in the patient. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is triple negative breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of identifying a breast cancer patient having increased risk of relapse or reduced survival comprising detecting a decreased expression level of at least one gene from Table 2, relative to a standard control, in a biological sample obtained from the patient, thereby identifying the patient as having increased risk of relapse or reduced survival. In embodiments, the method further comprises administering to the patient an effective amount of a chemotherapeutic therapy. The disclosure provides methods of selecting a breast cancer patient for treatment with a chemotherapeutic therapy comprising detecting a decreased expression level of at least one gene from Table 2, relative to a standard control, in a biological sample obtained from the patient; thereby selecting the breast cancer patient for treatment with the chemotherapeutic agent. In embodiments, the method further comprises administering to the patient an effective amount of a chemotherapeutic therapy. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is triple negative breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of treating breast cancer in a patient in need thereof comprising administering to the patient an effective amount of a chemotherapeutic therapy, wherein a biological sample obtained from the patient has a decreased expression level of at least one gene from Table 2, relative to a standard control. The disclosure provides methods of treating breast cancer in a patient in need thereof comprising: (i) detecting a decreased expression level of at least one gene from Table 2, relative to a standard control, in a biological sample obtained from the patient; and (ii) administering to the patient an effective amount of a chemotherapeutic therapy. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is triple negative breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

In embodiments, the patient has a decreased expression level of at least 2 genes from Table 2, relative to a control. In embodiments, the patient has a decreased expression level of at least 3 genes from Table 2, relative to a control. In embodiments, the patient has a decreased expression level of at least 4 genes from Table 2, relative to a control. In embodiments, the patient has a decreased expression level of at least 5 genes from Table 2, relative to a control. In embodiments, the patient has a decreased expression level of at least 6 genes from Table 2, relative to a control. In embodiments, the patient has a decreased expression level of at least 7 genes from Table 2, relative to a control. In embodiments, the patient has a decreased expression level of at least 8 genes from Table 2, relative to a control. In embodiments, the patient has a decreased expression level of at least 9 genes from Table 2, relative to a control. In embodiments, the patient has a decreased expression level of at least 10 genes from Table 2, relative to a control. In embodiments, the patient has a decreased expression level of at least 11 genes from Table 2, relative to a control. In embodiments, the patient has a decreased expression level of at least 12 genes from Table 2, relative to a control. In embodiments, the patient has a decreased expression level of at least 13 genes from Table 2, relative to a control. In embodiments, the patient has a decreased expression level of at least 14 genes from Table 2, relative to a control. In embodiments, the patient has a decreased expression level of at least 15 genes from Table 2, relative to a control. In embodiments, the patient has a decreased expression level of at least 16 genes from Table 2, relative to a control. In embodiments, the patient has a decreased expression level of at least 17 genes from Table 2, relative to a control. In embodiments, the patient has a decreased expression level of at least 18 genes from Table 2, relative to a control. In embodiments, the patient has a decreased expression level of at least 19 genes from Table 2, relative to a control. In embodiments, the patient has a decreased expression level of at least 20 genes from Table 2, relative to a control. In embodiments, the patient has a decreased expression level of at least 21 genes from Table 2, relative to a control. In embodiments, the patient has a decreased expression level of at least 22 genes from Table 2, relative to a control. In embodiments, the patient has a decreased expression level of at least 23 genes from Table 2, relative to a control. In embodiments, the patient has a decreased expression level of at least 24 genes from Table 2, relative to a control. In embodiments, the patient has a decreased expression level of at least 25 genes in Table 2, relative to a control. In embodiments, the patient has a decreased expression level of at least 26 genes in Table 2, relative to a control. In embodiments, the patient has a decreased expression level of at least 27 genes in Table 2, relative to a control. In embodiments, the patient has a decreased expression level of at least 28 genes in Table 2, relative to a control. In embodiments, the patient has a decreased expression level of at least 29 genes in Table 2, relative to a control. In embodiments, the patient has a decreased expression level of at least 30 genes in Table 2, relative to a control. In embodiments, the patient has a decreased expression level of at least 31 genes in Table 2, relative to a control. In embodiments, the patient has a decreased expression level of at least 32 genes in Table 2, relative to a control. In embodiments, the patient has a decreased expression level of at least 33 genes in Table 2, relative to a control. In embodiments, the patient has a decreased expression level of at least 34 genes in Table 2, relative to a control. In embodiments, the patient has a decreased expression level of at least 35 genes in Table 2, relative to a control. In embodiments, the patient has a decreased expression level of at least 36 genes in Table 2, relative to a control. In embodiments, the patient has a decreased expression level of at least 37 genes in Table 2, relative to a control. In embodiments, the patient has a decreased expression level of 2 genes, 3 genes, 4 genes, 5, genes, 6 genes, 7 genes, 8 genes, 9 genes, 10 genes, 11 genes, 12 genes, 13, genes, 14 genes, 15 genes, 16 genes, 17 genes, 18 genes, 19, genes, 20 genes, 21 genes, 22, genes, 23 genes, or 24 genes, 25 genes, 26 genes, 27 genes, 28 genes, 29 genes, 30 genes, 31 genes, 32 genes, 33 genes, 34 genes, 35 genes, 36 genes, or 37 genes, in Table 2, relative to a control. In embodiments, the patient has a decreased expression level of 38 genes in Table 2, relative to a control. In embodiments, the decreased expression level of the at least 2-38 genes from Table 2 comprises an unequally weighted average of the decreased expression level of the at least 2-38 genes from Table 2.

The disclosure provides methods of identifying a breast cancer patient having a good prognosis comprising detecting an increased expression level of at least one gene from Table 2 in a biological sample obtained from the patient, thereby identifying the patient as having a good prognosis. In embodiments, the method further comprises administering to the patient an effective amount of a non-chemotherapeutic therapy. The disclosure provides methods of selecting a breast cancer patient for treatment with a non-chemotherapeutic therapy comprising detecting an increased expression level of at least one gene from Table 2 in a biological sample obtained from the patient; thereby selecting the breast cancer patient for treatment with the non-chemotherapeutic therapy. In embodiments, the method further comprises administering to the patient an effective amount of a non-chemotherapeutic therapy. In embodiments, the non-chemotherapeutic therapy is hormone therapy. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is triple negative breast cancer and the patient is a postmenopausal woman. In embodiments, the breast cancer is ER+PR+ breast cancer and the patient is a postmenopausal woman. In embodiments, the breast cancer is triple negative breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of treating breast cancer in a patient in need thereof comprising administering to the patient an effective amount of a non-chemotherapeutic therapy, wherein a biological sample obtained from the patient has an increased expression level of at least one gene from Table 2. The disclosure provides methods of treating breast cancer in a patient in need thereof comprising: (i) detecting an increased expression level of at least one gene from Table 2 in a biological sample obtained from the patient; and (ii) administering to the patient an effective amount of a non-chemotherapeutic therapy. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is triple negative breast cancer and the patient is a postmenopausal woman. In embodiments, the breast cancer is ER+PR+ breast cancer and the patient is a postmenopausal woman. In embodiments, the breast cancer is triple negative breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

In embodiments, the patient has a increased expression level of at least 2 genes from Table 2, relative to a control. In embodiments, the patient has a increased expression level of at least 3 genes from Table 2, relative to a control. In embodiments, the patient has a increased expression level of at least 4 genes from Table 2, relative to a control. In embodiments, the patient has a increased expression level of at least 5 genes from Table 2, relative to a control. In embodiments, the patient has a increased expression level of at least 6 genes from Table 2, relative to a control. In embodiments, the patient has a increased expression level of at least 7 genes from Table 2, relative to a control. In embodiments, the patient has a increased expression level of at least 8 genes from Table 2, relative to a control. In embodiments, the patient has a increased expression level of at least 9 genes from Table 2, relative to a control. In embodiments, the patient has a increased expression level of at least 10 genes from Table 2, relative to a control. In embodiments, the patient has a increased expression level of at least 11 genes from Table 2, relative to a control. In embodiments, the patient has a increased expression level of at least 12 genes from Table 2, relative to a control. In embodiments, the patient has a increased expression level of at least 13 genes from Table 2, relative to a control. In embodiments, the patient has a increased expression level of at least 14 genes from Table 2, relative to a control. In embodiments, the patient has a increased expression level of at least 15 genes from Table 2, relative to a control. In embodiments, the patient has a increased expression level of at least 16 genes from Table 2, relative to a control. In embodiments, the patient has a increased expression level of at least 17 genes from Table 2, relative to a control. In embodiments, the patient has a increased expression level of at least 18 genes from Table 2, relative to a control. In embodiments, the patient has a increased expression level of at least 19 genes from Table 2, relative to a control. In embodiments, the patient has a increased expression level of at least 20 genes from Table 2, relative to a control. In embodiments, the patient has a increased expression level of at least 21 genes from Table 2, relative to a control. In embodiments, the patient has a increased expression level of at least 22 genes from Table 2, relative to a control. In embodiments, the patient has a increased expression level of at least 23 genes from Table 2, relative to a control. In embodiments, the patient has a increased expression level of at least 24 genes from Table 2, relative to a control. In embodiments, the patient has a increased expression level of at least 25 genes in Table 2, relative to a control. In embodiments, the patient has a increased expression level of at least 26 genes in Table 2, relative to a control. In embodiments, the patient has a increased expression level of at least 27 genes in Table 2, relative to a control. In embodiments, the patient has a increased expression level of at least 28 genes in Table 2, relative to a control. In embodiments, the patient has a increased expression level of at least 29 genes in Table 2, relative to a control. In embodiments, the patient has a increased expression level of at least 30 genes in Table 2, relative to a control. In embodiments, the patient has a increased expression level of at least 31 genes in Table 2, relative to a control. In embodiments, the patient has a increased expression level of at least 32 genes in Table 2, relative to a control. In embodiments, the patient has a increased expression level of at least 33 genes in Table 2, relative to a control. In embodiments, the patient has a increased expression level of at least 34 genes in Table 2, relative to a control. In embodiments, the patient has a increased expression level of at least 35 genes in Table 2, relative to a control. In embodiments, the patient has a increased expression level of at least 36 genes in Table 2, relative to a control. In embodiments, the patient has a increased expression level of at least 37 genes in Table 2, relative to a control. In embodiments, the patient has a increased expression level of 2 genes, 3 genes, 4 genes, 5, genes, 6 genes, 7 genes, 8 genes, 9 genes, 10 genes, 11 genes, 12 genes, 13, genes, 14 genes, 15 genes, 16 genes, 17 genes, 18 genes, 19, genes, 20 genes, 21 genes, 22, genes, 23 genes, or 24 genes, 25 genes, 26 genes, 27 genes, 28 genes, 29 genes, 30 genes, 31 genes, 32 genes, 33 genes, 34 genes, 35 genes, 36 genes, or 37 genes, in Table 2, relative to a control. In embodiments, the patient has a increased expression level of 38 genes in Table 2, relative to a control. In embodiments, the increased expression level of the at least 2-38 genes from Table 2 comprises an unequally weighted average of the increased expression level of the at least 2-38 genes from Table 2.

In embodiments, the disclosure provides a method of identifying a breast cancer patient having increased risk of relapse or reduced survival, the method comprising detecting an elevated level of exhausted CD8+ T cells, relative to a standard control, in a biological sample obtained from the patient and detecting a decreased level of CD26+CD4+ T cells, relative to a standard control, in a biological sample obtained from the patient; wherein the elevated level of exhausted CD8+ T cells and the decreased level of CD26+CD4+ T cells indicates the patient as having increased risk of relapse or reduced survival. In embodiments, the method further comprises administering to the patient an effective amount of a chemotherapeutic therapy. In embodiments, the disclosure provides a method of identifying a breast cancer patient for treatment with a chemotherapeutic therapy comprising detecting an elevated level of exhausted CD8+ T cells, relative to a standard control, in a biological sample obtained from the patient and detecting a decreased level of CD26+CD4+ T cells, relative to a standard control, in a biological sample obtained from the patient; thereby identifying the patient for treatment with a chemotherapeutic therapy. In embodiments, the method further comprises administering to the patient an effective amount of a chemotherapeutic therapy. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

In embodiments, the disclosure provides a method of treating breast cancer in a patient in need thereof comprising administering to the patient an effective amount of a chemotherapeutic therapy, wherein a biological sample obtained from the patient has an elevated level of exhausted CD8+ T cells relative to a standard control and a decreased level of CD26+CD4+ T cells relative to a standard control. In embodiments, the disclosure provides a method of treating breast cancer in a patient in need thereof comprising: (i) detecting an elevated level of exhausted CD8+ T cells, relative to a standard control, in a biological sample obtained from the patient; (ii) detecting a decreased level of CD26+CD4+ T cells in a biological sample obtained from the patient; and (iii) administering to the patient an effective amount of a chemotherapeutic therapy. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of treating breast cancer in a patient in need thereof comprising (i) detecting a expression level of at least one gene from Table 1 and at least one gene from Table 2 in a biological sample obtained from the patient; (ii) identifying the expression level of the at least one gene from Table 1 as being higher than a standard control or as being lower than a standard control; (iii) identifying the expression level of the at least one gene from Table 2 as being higher than a standard control or as being lower than a standard control; and (iv) administering to the patient: (a) an effective amount of a non-chemotherapeutic therapy when the expression level of the at least one gene from Table 1 is lower than the standard control and the expression level of the at least one gene from Table 2 is greater than the standard control, thereby treating the breast cancer in the patient; or (b) an effective amount of a chemotherapeutic therapy when the expression level of the at least one gene from Table 1 is greater than the standard control and the expression level of the at least one gene from Table 2 is lower than the standard control, thereby treating the breast cancer in the patient. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer. In embodiments, the breast cancer is triple negative breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of treating breast cancer in a patient in need thereof comprising (i) detecting a expression level of at least one gene from Table 1 and at least one gene from Table 2 in a biological sample obtained from the patient; (ii) identifying the expression level of the at least one gene from Table 1 as being higher than a standard control; (iii) identifying the expression level of the at least one gene from Table 2 as being lower than a standard control; and (iv) administering to the patient an effective amount of a chemotherapeutic therapy, thereby treating the breast cancer in the patient. The disclosure provides methods of treating breast cancer in a patient in need thereof comprising (i) detecting a expression level of at least one gene from Table 1 and at least one gene from Table 2 in a biological sample obtained from the patient; (ii) identifying the expression level of the at least one gene from Table 1 as being lower than a standard control; (iii) identifying the expression level of the at least one gene from Table 2 as being higher than a standard control; and (iv) administering to the patient an effective amount of a non-chemotherapeutic therapy, thereby treating the breast cancer in the patient. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

The disclosure provides methods of predicting the risk of relapse or reduced survival in a breast cancer patient comprising (i) detecting a expression level of at least one gene from Table 1 and at least one gene from Table 2 in a biological sample obtained from the patient; (ii) identifying the expression level of the at least one gene from Table 1 as being greater than a standard control or as being lower than a standard control; (ii) identifying the expression level of the at least one gene from Table 2 as being greater than a standard control or as being lower than a standard control; and (iii) predicting increased risk of relapse or reduced survival when the expression level of the at least one gene from Table 1 is greater than the standard control and the expression level of the at least one gene from Table 2 is lower than the standard control; and predicting a good prognosis when the expression level of the at least one gene from Table 1 is reduced relative to the standard control and the expression level of the at least one gene from Table 2 is greater than the standard control. In embodiments, the breast cancer is ER+ breast cancer and the patient is a premenopausal woman. In embodiments, the breast cancer is PR+ breast cancer and the patient is a premenopausal woman. In embodiments, the breast cancer is ER+PR+ breast cancer and the patient is a premenopausal woman. In embodiments, the breast cancer is ER+ breast cancer and the patient is a postmenopausal woman. In embodiments, the breast cancer is PR+ breast cancer and the patient is a postmenopausal woman. In embodiments, the breast cancer is ER+PR+ breast cancer and the patient is a postmenopausal woman. In embodiments, the breast cancer is ER+ breast cancer. In embodiments, the breast cancer is PR+ breast cancer. In embodiments, the breast cancer is ER+ and PR+ breast cancer. In embodiments, the patient is a premenopausal woman. In embodiments, the patient is a postmenopausal woman. In embodiments, the biological sample is a tumor. In embodiments, the biological sample is tumor tissue. In embodiments, the biological sample is tumor cells. In embodiments, the tumor is a primary tumor.

In embodiments of the methods described herein, the at least one gene from Table 1 is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or 25 genes from Table 1. In embodiments, the at least one gene from Table 1 is 25 genes from Table 1. In embodiments, the increased expression level of the at least 2-25 genes from Table 1 comprises an unequally weighted average of the increased expression level of the 2-25 genes from Table 1. In embodiments, the increased expression level of the 25 genes from Table 1 comprises an unequally weighted average of the increased expression level of the 25 genes from Table 1. In embodiments, the decreased expression level of the at least 2-25 genes from Table 1 comprises an unequally weighted average of the decreased expression level of the 2-25 genes from Table 1. In embodiments, the decreased expression level of the 25 genes from Table 1 comprises an unequally weighted average of the decreased expression level of the 25 genes from Table 1.

In embodiments of the methods described herein, the at least one gene from Table 2 is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, or 38 genes from Table 2. In embodiments, the at least one gene from Table 2 is 38 genes from Table 2. In embodiments, the increased expression level of the at least 2-38 genes from Table 2 comprises an unequally weighted average of the increased expression level of the 2-38 genes from Table 2. In embodiments, the increased expression level of the 38 genes from Table 2 comprises an unequally weighted average of the increased expression level of the 38 genes from Table 2. In embodiments, the decreased expression level of the at least 2-38 genes from Table 2 comprises an unequally weighted average of the decreased expression level of the 2-38 genes from Table 2. In embodiments, the decreased expression level of the 38 genes from Table 2 comprises an unequally weighted average of the decreased expression level of the 38 genes from Table 2.

Detection

The methods of detecting the levels of CD8+ TEX, the expression levels of the genes in Table 1, the levels of CD26+CD4+ T cells, and the expression levels of the genes in Table 2 can be conducted by any method known in the art. In embodiments, the methods comprise detecting RNA expression levels. In embodiments, the methods comprise detecting mRNA expression levels. In embodiments, the methods comprise detecting protein expression levels.

RNA may be detected by any known methodology, including but not limited to rtPCR, RNA sequencing, nanopore sequencing, microarray, hybridization-based sequencing, hybridization-based detection and quantification (e.g., NanoString).

Protein may be detected by any known methodology, including but not limited to high-performance liquid chromatography (HPLC); mass spectrometry (MS), e.g., Liquid chromatography-mass spectrometry; Enzyme-linked immunosorbent assay (ELISA); Protein immunoprecipitation; immunoelectrophoresis; Western blot; protein immunostaining; immunofluorescence; mass cytometry; immunohistochemistry.

In embodiments, detecting the expression level of the one or more genes includes calculating the mean of Log 2 of the expression of the one or more genes in a biological sample. In an embodiment, gene expression is determined by Nanostring counts. In one embodiment, gene expression is determined by number of transcripts detected in the sample. One skilled in the art could use other methods for quantifying gene expression (e.g., mRNA levels), such as RNAseq or quantitative PCR.

In embodiments, detecting the expression level of the one or more proteins includes calculating the mean of Log 2 of the amount of the one or more proteins in a biological sample. The resulting value can then be compared to other values obtained in the same manner (e.g., based on level of the same proteins in a control). In embodiments, protein level is determined by immunohistochemistry (IHC), flow cytometry, high-performance liquid chromatography (HPLC),mass spectrometry (MS), HPC/MS, enzyme-linked immunosorbent assay (ELISA); protein immunoprecipitation, immunoelectrophoresis, Western blot, protein immunostaining, immunofluorescence, or mass cytometry. In embodiments, protein level is determined by immunohistochemistry (IHC).

In embodiments, the expression level of the one or more genes comprises an average of the expression levels of the one or more genes. In embodiments, the expression level of the one or more genes comprises an unequally weighted average of the expression levels of the one or more genes. When the expression level of the gene is calculated as the mean of Log 2 of the amount, then the expression level of each gene is weighted based on the mean Log 2 calculation for that gene. As such, when the average of the expression level is calculated, the average of the expression levels are unequally weighted.

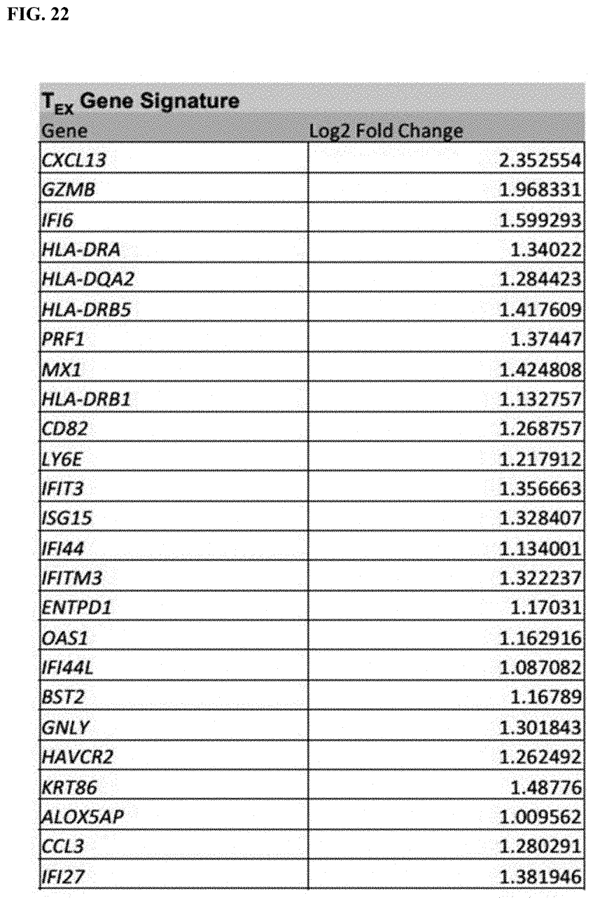

In embodiments, the expression level of each gene is multiplied by its corresponding weight (i.e., the Log 2 Fold Change value shown in FIG. 22). Then the products of weights and expression levels of the genes in Table 1 are summed, and the product sum is divided by the sum of the weights. This divided sum is the weighted average of RNA expression levels (aka, T_{EX} signature score).

Chemotherapeutic Therapy

In embodiments of the methods described herein, the patient is administered an effective amount of a chemotherapeutic therapy. “Chemotherapeutic therapy” refers to any compound, composition, combination therapy, treatment plan, or treatment regimen that comprises a chemotherapeutic agent and optionally a non-chemotherapeutic agent. In embodiments, chemotherapeutic therapy comprises a chemotherapeutic agent and a non-chemotherapeutic agent. In embodiments, the non-chemotherapeutic agent is hormone therapy, immunotherapy, targeted therapy, radiation, gene therapy or a combination of two or more thereof. In embodiments, the non-chemotherapeutic agent is hormone therapy, immunotherapy, targeted therapy, radiation, or a combination of two or more thereof. In embodiments, chemotherapeutic therapy comprises a chemotherapeutic agent and at least one treatment selected from the group consisting of hormone therapy, immunotherapy, targeted therapy, radiation, and gene therapy. In embodiments, chemotherapeutic therapy comprises a chemotherapeutic agent and at least one treatment selected from the group consisting of hormone therapy, immunotherapy, targeted therapy, and radiation. In embodiments, chemotherapeutic therapy comprises a chemotherapeutic agent and at least one treatment selected from the group consisting of hormone therapy, immunotherapy, and radiation.

“Chemotherapeutic agent” is used in accordance with its plain ordinary meaning and refers to a chemical composition or compound having antineoplastic properties or the ability to inhibit the growth or proliferation of cells. Chemotherapeutic agents are well-known in the art and commercially available.

In embodiments of the methods described herein, the chemotherapeutic agent is an alkylating agent, antimetabolite compound, anthracycline compound, antitumor antibiotic, a platinum compound, a topoisomerase inhibitor, a vinca alkaloid, a taxane compound, or an epothilone compound. In embodiments, the chemotherapeutic agent is an alkylating agent. In embodiments, the alkylating agent is carboplatin, chlorambucil, cyclophosphamide, melphalan, mechlorethamine, procarbazine, or thiotepa. In embodiments, the chemotherapeutic agent is antimetabolite compound. In embodiments, the antimetabolite compound is azacitidine, capecitabine, cytarabine, gemcitabine, doxifluridine, hydroxyurea, methotrexate, pemetrexed, 6-thioguanine, 5-fluorouracil, or 6-mercaptopurine. In embodiments, the chemotherapeutic agent is anthracycline compound. In embodiments, the anthracylince compound is daunorubicin, doxorubicin, idarubicin, epirubicin, or mitoxantrone. In embodiments, the chemotherapeutic agent is antitumor antibiotic. In embodiments, the antitumor antibiotic is actinomycin, bleomycin, mitomycin, or valrubicin. In embodiments, the chemotherapeutic agent is a platinum compound. In embodiments, the platinum compound is cisplatin or oxaliplatin. In embodiments, the chemotherapeutic agent is a topoisomerase inhibitor. In embodiments, the topoisomerase inhibitor is irinotecan, topotecan, amscarine, etoposide, teniposide, or eribulin. In embodiments, the chemotherapeutic agent is a vinca alkaloid. In embodiments, the vinca alkaloid is vincristine, vinblastine, vinorelbine, or vindesine. In embodiments, the chemotherapeutic agent is a taxane compound. In embodiments, the taxane compound is paclitaxel or docetaxel. In embodiments, the chemotherapeutic agent is an epothilone compound. In embodiments, the epothiolone compound is epothilone, ixabepilone, patupilone, or sagopilone. In embodiments, the chemotherapeutic agent is carboplatin, chlorambucil, cyclophosphamide, melphalan, mechlorethamine, procarbazine, thiotepa, azacitidine, capecitabine, cytarabine, gemcitabine, doxifluridine, hydroxyurea, methotrexate, pemetrexed, 6-thioguanine, 5-fluorouracil, 6-mercaptopurine, daunorubicin, doxorubicin, idarubicin, epirubicin, mitoxantrone, actinomycin, bleomycin, mitomycin, valrubicin, cisplatin, oxaliplatin, irinotecan, topotecan, amscarine, etoposide, teniposide, eribulin, vincristine, vinblastine, vinorelbine, vindesine, paclitaxel, docetaxel, epithilone, ixabepilone, patupilone, and sagopilone

Non-Chemotherapeutic Therapy

In embodiments of the methods described herein, the patient is administered an effective amount of a non-chemotherapeutic therapy. “Non-chemotherapeutic therapy” refers to any compound, composition, combination therapy, treatment plan, or treatment regimen that does not include a chemotherapeutic agent. In embodiments, the non-chemotherapeutic therapy comprises hormone therapy, immunotherapy, targeted therapy, radiation, gene therapy, or a combination of two or more thereof. In embodiments, the non-chemotherapeutic therapy comprises hormone therapy. In embodiments, the non-chemotherapeutic therapy comprises hormone therapy and immunotherapy. In embodiments, the non-chemotherapeutic therapy comprises hormone therapy, immunotherapy, and targeted therapy. In embodiments, the non-chemotherapeutic therapy comprises hormone therapy, immunotherapy, targeted therapy, and radiation. In embodiments, the non-chemotherapeutic therapy comprises immunotherapy. In embodiments, the non-chemotherapeutic therapy comprises targeted therapy.

“Hormone therapy” is used in accordance with its plain and ordinary meaning and refers to any drug that can lower the amount of hormones in the body or that can block hormones from accessing breast cancer cells. In embodiments, hormone therapy comprises a selective estrogen receptor modulator (SERM), a selective estrogen receptor degrader (SERD), an aromatase inhibitor, a luteinizing hormone-releasing hormone (LHRH) agonist, or a combination of two or more thereof. In embodiments, hormone therapy comprises a selective estrogen receptor modulator. In embodiments, hormone therapy comprises a selective estrogen receptor degrader. In embodiments, hormone therapy comprises an aromatase inhibitor. In embodiments, hormone therapy comprises a luteinizing hormone-releasing hormone agonist. In embodiments, hormone therapy comprises a selective estrogen receptor modulator and a luteinizing hormone-releasing hormone (LHRH) agonist. In embodiments, the selective estrogen receptor modulator is tamoxifen, toremifene, raloxifene, lasofoxifene, bazedoxifen, ospemifene, anordrin, broparestrol, clomifene, cyclofenil, ormeloxifene, acolbifene, afimoxifene, elacestrant, enclomifene, endoxifen, or zuclomifene. In embodiments, the selective estrogen receptor modulator is tamoxifen. In embodiments, the selective estrogen receptor degrader is fulvestrant, brilanestrant, giredestrant, amcenestrant, camizestrant, rintodestrant, LSZ102, LY3484356, elecestrant, or ZN-c5. In embodiments, the selective estrogen receptor degrader is fulvestrant. In embodiments, the aromatase inhibitor is letrozole, anastrozole, exemastane, or testolactone. In embodiments, the luteinizing hormone-releasing hormone agonist is goserelin, leuprolide, histrelin, or triptorelin.

“Immunotherapy” refers an drug or treatment that stimulates the patient's own immune system. In embodiments, the immunotherapy is a PD-1 inhibitor, a PD-L1 inhibitor, a CTLA-4 inhibitor, a LAG-3 inhibitor, a TIM-3 inhibitor, an adenosine A2A receptor antagonist, and the like. In embodiments, the immunotherapy is atezolizumab, dostarlimab, or pembrolizumab.

“Targeted therapy” refers to a drug or treatment that directly targets proteins on breast cancer cells. In embodiments, targeted therapy is a CDK4/6 inhibitor. In embodiments, the CD4/6 inhibitor is palbociclib, ribociclib, or abemaciclib. In embodiments, “non-chemotherapeutic therapy” comprises an aromatase inhibitor and a CDK4/6 inhibitor. In embodiments, “non-chemotherapeutic therapy” comprises a selective estrogen receptor degrader and a CDK4/6 inhibitor.

“Radiation” or “radiation therapy” refers to any type of radiation used to treat breast cancer. In embodiments, the radiation is external beam radiation or brachytherapy/internal radiation.

A “effective amount”, as used herein, is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” or “combined effective amount” is an amount sufficient to contribute to the treatment, prevention, or reduction of a symptom or symptoms of a disease, which could also be referred to as a “therapeutically effective amount” or “therapeutically combined effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

Kits

Provided herein are kits comprising one or more reagents capable of detecting the expression level of one or more genes set forth in Table 1 and/or Table 2. The kit may comprise an assay system including any one of assay reagents, assay controls, protocols, exemplary assay results, or combinations of these components designed to provide the user with means to evaluate the expression level of the genes in the present disclosure. In another aspect, the disclosure provides a kit for diagnosing breast cancer in an individual including reagents for detecting at least one gene from Table 1 and/or Table 2 in a biological sample from a subject.

In embodiments, the kits comprise one or more of the following: an antibody, wherein the antibody specifically binds with a protein expressed by the genes described herein, a labeled binding partner to the antibody, a solid phase upon which is immobilized the antibody or its binding partner, an RNA probe that can hybridize to a mRNA expressed by the genes described herein, pairs of primers that under appropriate reaction conditions can prime amplification of at least a portion of a mRNA expressed by the genes described herein or RNA encoding a protein (e.g., by PCR), instructions on how to use the kit, and a label or insert indicating regulatory approval for diagnostic or therapeutic use.

In embodiments, the disclosure further includes RNA or protein microarrays comprising proteins of the disclosure (e.g., proteins expressed by the genes), RNA of the disclosure (e.g., mRNA expressed by the genes), or molecules, such as antibodies, which specifically bind to the proteins or RNA of the present disclosure. In embodiments, standard techniques of microarray technology are utilized to assess expression of the proteins and/or identify biological constituents that bind such proteins. Protein microarray technology is well known to those of ordinary skill in the art and is based on, but not limited to, obtaining an array of identified proteins on a fixed substrate, binding target molecules or biological constituents to the proteins, and evaluating such binding. RNA arrays also can be used for diagnostic applications, such as for identifying subjects that have a condition characterized by expression of mRNA and proteins, e.g., breast cancer.

In embodiments, the assay systems of the disclosure include a means for detecting in a sample of tumor cells a level of amplification of the genes. In embodiments, the assay system includes one or more controls. The controls may include: (i) a control sample for detecting breast cancer in an individual; (ii) a control sample for detecting the absence of breast cancer; and (iii) information containing a predetermined control level of gene markers to be measured with regard to the diagnosis of or progression of breast cancer.

In embodiments, a means for detecting the expression level of the genes of the disclosure can be any type of reagent that can include, but are not limited to, antibodies and antigen binding fragments thereof, peptides, binding partners, aptamers, enzymes, and small molecules. Additional reagents useful for performing an assay using such means for detection can also be included, such as reagents for performing immunohistochemistry or another binding assay.

The means for detecting of the assay system of the present disclosure can be conjugated to a detectable tag or detectable label. Such a tag can be any suitable tag which allows for detection of the reagents used to detect the gene or protein of interest and includes, but is not limited to, any composition or label detectable by spectroscopic, photochemical, electrical, optical or chemical means. Useful labels in the present disclosure include: biotin for staining with labeled streptavidin conjugate, magnetic beads (e.g., Dynabeads), fluorescent dyes (e.g., fluorescein, Texas red, rhodamine, green fluorescent protein, and the like), radiolabels (e.g., ³H, ¹²⁵I, ³⁵S, ¹⁴C, or ³²P) enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

In addition, the means for detecting of the assay system of the present disclosure can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide). The kit can also include suitable reagents for the detection of the reagent and/or for the labeling of positive or negative controls, wash solutions, dilution buffers and the like. The assay system can also include a set of written instructions for using the system and interpreting the results.

The assay system can also include a means for detecting a control marker that is characteristic of the cell type being sampled can generally be any type of reagent that can be used in a method of detecting the presence of a known gene (at the nucleic acid or protein level) in a sample, such as by a method for detecting the presence of a gene of Table 1 and/or Table 2. In embodiments, the means is characterized in that it identifies a specific marker of the cell type being analyzed that positively identifies the cell type. For example, in breast cancer assay, it is desirable to screen breast cancer cells for the level of the gene expression and/or biological activity. Therefore, the means for detecting a control marker identifies a marker that is characteristic of a breast cancer cell, so that the cell is distinguished from other cell types. Such a means increases the accuracy and specificity of the assay of the present disclosure. Such a means for detecting a control marker include, but are not limited to: a probe that hybridizes under stringent hybridization conditions to a nucleic acid molecule encoding a protein marker; PCR primers which amplify such a nucleic acid molecule; an aptamer that specifically binds to a conformationally-distinct site on the target molecule; and/or an antibody, antigen binding fragment thereof, or antigen binding peptide that selectively binds to the control marker in the sample. Nucleic acid and amino acid sequences for many cell markers are known in the art and can be used to produce such reagents for detection.

Provided herein are kits comprising one or more reagents capable of detecting the expression level of one or more genes set forth in Table 1. Provided herein are kits comprising one or more reagents capable of detecting the expression level of one or more genes set forth in Table 2. Provided herein are kits comprising one or more reagents capable of detecting the expression level of one or more genes set forth in Table 1 and Table 2. In embodiments, the kits contain reagents to detect mRNA expression levels of the one or more genes set forth in Table 1, Table 2, or Table 1 and Table 2. In embodiments, the kits contain reagents to detect protein expression levels of the one or more genes set forth in Table 1, Table 2, or Table 1 and Table 2. In embodiments, the reagent is capable of detecting: (i) mRNA expressed by the genes set forth in Table 1, (ii) mRNA expressed by the genes set forth in Table 2, (iii) mRNA expressed by the genes set forth in Table 1 and Table 2, (iv) protein expressed by the genes set forth in Table 1, (v) protein expressed by the genes set forth in Table 2, (vi) protein expressed by the genes set forth in Table 1 and Table 2, (vii) mRNA and protein expressed by the genes set forth in Table 1, (ii) mRNA and protein expressed by the genes set forth in Table 2, (iii) mRNA and protein expressed by the genes set forth in Table 1 and Table 2. The one or more reagents may be detection agents, probes, primers, solid supports, standard controls, and other materials capable of detecting and/or identifying and/or quantifying mRNA and/or proteins. In embodiments, the kit comprises instructions for use. In embodiments, the kit further comprises a chemotherapeutic therapy, a non-chemotherapeutic therapy, or a combination thereof.

“Detection agent” refers to (i) a compound that is capable of binding (covalently or non-covalently) a protein and (ii) a detectable label. The “detection agent” can be an “indirect detection agent” or a “direct detection agent.” An “indirect detection agent” refers to a compound that is capable of binding (covalently or non-covalently) a protein that cannot be detected itself, but is detected using a separate, distinct detectable label that specifically binds (covalently or non-covalently) to the compound that is capable of binding the protein. A “direct detection agent” refers to a compound that is capable of binding (covalently or non-covalently) a protein and is also a detectable label (e.g., whether the compound and detectable label are the same compound or whether the compound and detectable label are separate, bound compounds). Exemplary detection agents that can interact with a protein include antibodies (monoclonal or polyclonal), RNA, DNA, biotin, and the like. In one embodiment, the detection agent that interacts with the protein is, or includes, antibody. In embodiments, the detection agent comprises antibody bound to an enzyme. In embodiments, the detection agent includes a primary antibody bound to a secondary antibody that is bound to an enzyme. In embodiments, the detection agent comprises biotin and streptavidin. In embodiments, the detection agent comprises biotin, streptavidin, and an enzyme. In embodiments, the detection agent comprises biotin and avidin. In embodiments, the detection agent comprises biotin, avidin, and an enzyme.

“Detectable label” refers to a moiety that indicates the presence of a corresponding molecule to which it is bound. A “detectable label” can be an indirect or direct label. An “indirect label” refers to a moiety, or ligand, that is detected using a labeled secondary agent, or ligand-binding partner, that specifically binds to the indirect label. A “direct label” refers to a moiety that is detectable in the absence of a ligand-binding partner interaction. Exemplary detectable labels include fluorescent labels (including, e.g., quenchers or absorbers), non-fluorescent labels, colorimetric labels, chemiluminescent labels, bioluminescent labels, radioactive labels (such as ³H, ³5S, ³²P, ¹²⁵I, ⁵⁷Co or ¹⁴C), mass-modifying groups, antibodies, antigens, biotin, haptens, digoxigenin, enzymes (including, e.g., peroxidase, phosphatase, etc.), and the like.

“Detectable complex” refers to a composition comprising (i) a detection agent and (ii) a protein, where the detection agent and protein are bound (covalently or non-covalently) together, and where the detectable complex can be identified and/or quantified by methods known in the art.

“Probe” refers to a nucleotide that includes a target-binding region that is substantially complementary to a target sequence in a target nucleic acid (e.g., mRNA expression sequence) and, thus, is capable of forming a hydrogen-bonded duplex with the target nucleic acid. Typically, the probe is a single-stranded probe, having one or more detectable labels to permit the detection of the probe following hybridization to its complementary target.

“Hybridization complex” refers to a composition containing (i) a probe and (ii) a target nucleic acid, where the probe and target nucleic acid are bound (e.g., hybridized) together, and where the hybridization complex can be identified and/or quantified by methods known in the art. The “target nucleic acid” refers to an mRNA expression sequence.

“Complementary” refers to sequence complementarity between two different nucleic acid strands or between two regions of the same nucleic acid strand. A first region of a nucleic acid is complementary to a second region of the same or a different nucleic acid if, when the two regions are arranged in anti-parallel fashion, at least one nucleotide residue of the first region is capable of base pairing (i.e., hydrogen bonding) with a residue of the second region, thus forming a hydrogen-bonded duplex.

“Substantially complementary” refers to two nucleic acid strands (e.g., a strand of a target nucleic acid and a complementary single-stranded oligonucleotide probe) that are capable of base pairing with one another to form a stable hydrogen-bonded duplex under stringent hybridization conditions, including the isothermal hybridization conditions described herein. In general, “substantially complementary” refers to two nucleic acids having at least 75%, for example, about 80%, 85%, 90%, 95%, 97%, 98%, 99% or 100% complementarity. The term “stringent” refers to hybridization conditions that affect the stability of hybrids, e.g., temperature, salt concentration, pH, formamide concentration, and the like. These conditions are empirically optimized to maximize specific binding, and minimize nonspecific binding, of a probe to a target nucleic acid (e.g., RNA).

Hybridization assays are well known in the art, and include, for example, sandwich hybridization assays, competitive hybridization assays, hybridization-ligation assays, dual ligation hybridization assays, nuclease hybridization assays, and the like. Nucleic acids “hybridize” when they associate, typically in solution. Nucleic acids hybridize due to a variety of well-characterized physicochemical forces, such as hydrogen bonding, solvent exclusion, base stacking and the like. In certain embodiments, hybridization occurs under conventional hybridization conditions, such as under stringent conditions as described, for example, in Sambrook et al., in “Molecular Cloning: A Laboratory Manual” (1989), Eds. J. Sambrook, E. F. Fritsch and T. Maniatis, Cold Spring Harbour Laboratory Press, Cold Spring Harbour, N.Y., which is incorporated by reference. Such conditions are, for example, hybridization in 6×SSC, pH 7.0/0.1% SDS at about 45° C. for 18-23 hours, followed by a washing step with 2×SSC/1% SDS at 50° C. In order to select the stringency, the salt concentration in the washing step can, for example, be chosen between 2×SSC/0.1% SDS at room temperature for low stringency and 0.2×SSC/0.1% SDS at 50° C. for high stringency. In addition, the temperature of the washing step can be varied between room temperature (ca. 22° C.), for low stringency, and 65° C. to 70° C. for high stringency. Also contemplated are polynucleotides that hybridize at lower stringency hybridization conditions. Changes in the stringency of hybridization and signal detection are primarily accomplished through the manipulation of, e.g., formamide concentration (lower percentages of formamide result in lowered stringency), salt conditions, or temperature. For example, lower stringency conditions include an overnight incubation at 37° C. in a solution comprising 6×SSPE (20×SSPE=3M NaCl; 0.2M NaH₂PO₄; 0.02M EDTA, pH 7.4), 0.5% SDS, 30% formamide, 100 mg/mL salmon sperm blocking DNA, followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. The inclusion of specific blocking reagents may require modification of the hybridization conditions described herein, due to problems with compatibility. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, (1997), which are each incorporated by reference. Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide details on the synthesis; labeling, detection and quantification of DNA and RNA, including oligonucleotides. Both Hames and Higgins 1 and 2 are incorporated by reference.

“Nucleic acid” refers to a polymer having multiple nucleotide monomers. “Nucleic acid” includes mRNA expression sequences. A nucleic acid can be single- or double-stranded, and can be DNA (e.g., cDNA or genomic DNA), RNA, or hybrid polymers (e.g., DNA/RNA). Nucleic acids can be chemically or biochemically modified and/or can contain non-natural or derivatized nucleotide bases. Nucleic acid modifications include, for example, methylation, substitution of one or more of the naturally occurring nucleotides with analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). Nucleic acids also include synthetic molecules that mimic nucleic acids in their ability to bind to a designated sequence via hydrogen bonding and other chemical interactions. Typically, the nucleotide monomers are linked via phosphodiester bonds, although synthetic forms of nucleic acids can comprise other linkages (e.g., peptide nucleic acids). Nucleic acids can also include, for example, conformationally restricted nucleic acids (e.g., locked nucleic acids).

“DNA” and “RNA” refer to deoxyribonucleic acid and ribonucleic acid, respectively.

Where a method disclosed herein refers to “amplifying” a nucleic acid, the term “amplifying” refers to a process in which the nucleic acid is exposed to at least one round of extension, replication, or transcription in order to increase (e.g., exponentially increase) the number of copies (including complimentary copies) of the nucleic acid. The process can be iterative including multiple rounds of extension, replication, or transcription. Various nucleic acid amplification techniques are known in the art, such as PCR amplification or rolling circle amplification.

A “primer” as used herein refers to a nucleic acid that is capable of hybridizing to a complimentary nucleic acid sequence in order to facilitate enzymatic extension, replication or transcription.

The terms “identical” or percent “identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

A variety of methods of specific DNA and RNA measurements that use nucleic acid hybridization techniques are known to those of skill in the art (see, Sambrook, Id.). Some methods involve electrophoretic separation (e.g., Southern blot for detecting DNA, and Northern blot for detecting RNA), but measurement of DNA and RNA can also be carried out in the absence of electrophoretic separation (e.g., quantitative PCR, dot blot, or array).

The sensitivity of the hybridization assays may be enhanced through use of a nucleic acid amplification system that multiplies the target nucleic acid being detected. Amplification can also be used for direct detection techniques. Examples of such systems include the polymerase chain reaction (PCR) system and the ligase chain reaction (LCR) system. Other methods include the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including TAQMAN® and molecular beacon probes can be used to monitor amplification reaction products in real time.

“Solid support” refers to a physical structure which can bind detection agents, probes, analytes, and/or reagents, covalently or non-covalently, in a device or method disclosed herein and embodiments thereof. Use of solid supports can facilitate detection and/or separation of analytes, e.g., splice isoforms, proteins coded by splice isoforms, RNA, nucleic acids, and the like. The choice of solid support for use in the present devices and methods is based upon the desired assay format and performance characteristics. Acceptable solid supports for use in the present devices and methods can vary widely. A solid support can be porous or nonporous. It can be continuous or non-continuous, and flexible or nonflexible. A solid support can be made of a variety of materials including ceramic, glass, silicon, metal, organic polymeric materials, or combinations thereof. In embodiments, the solid support is a resin or a bead. In embodiments, antibody can be immobilized on a solid support, e.g., magnetic or chromatographic matrix particles, the surface of an assay plate (e.g., microtiter wells), pieces of a solid substrate material or membrane (e.g., plastic, nylon, paper), and the like. In embodiments, the solid support is a micro-titer plate. In embodiments, the micro-titer plate is a polystyrene micro-titer plate. In embodiments, the solid support can be a microchip upon which nucleic acid reagent is affixed. In embodiments, binding of a portion of analyte (e.g., splice isoform sample) to a nucleic acid reagent affixed on a microchip results in formation of a detectable duplex nucleic acid. In embodiments, the solid support is a nitrocellulose or PVDF membrane. In embodiments, the solid support includes a protein binding surface which can be a microtiter plate, a colloidal metal particle, an iron oxide particle, a latex particle, a polymeric bead, and any combination thereof. In embodiments, antibodies or other polypeptides can be immobilized onto a solid support for use in assays. Solid phases that may be used to immobilize specific binding members include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TENTAGEL®, AGROGEL®, PEGA® gels, SPOCC® gels, and multiple-well plates.

### EXAMPLES

The following examples are for purposes of illustration and are not intended to limit the spirit or scope of the disclosure or claims.

### Example 1

CD8+ tumor infiltrating lymphocytes (TTLs) are associated with improved survival in triple negative breast cancer (TNBC), yet have no association with survival in estrogen receptor-positive (ER+) BC. The basis for these contrasting findings remains elusive. We identify subsets of BC tumors infiltrated by CD8⁺ T cells with characteristic features of exhausted T cells (T_{EX}). Tumors with abundant CD8⁺ T_{EX} exhibit a distinct tumor microenvironment marked by amplified interferon-γ signaling related pathways and higher PD-L1 expression. Paradoxically, higher levels of CD8⁺ T_{EX} TILs associate with decreased overall survival ER+BC patients, but not TNBC patients. Moreover, high tumor expression of a CD8⁺ T_{EX} signature identifies dramatically reduced survival in premenopausal, but not postmenopausal, ER+BC patients. Finally, we demonstrate the value of a tumor T_{EX} signature score in identifying high-risk premenopausal ER+BC patients amongst those with intermediate Oncotype Dx® breast recurrence scores. Our data highlight the complex relationship between CD8⁺ TILs, interferon-γ signaling, and estrogen receptor status in BC patient survival. This work identifies tumor infiltrating CD8⁺ T_{EX} as a key feature of early-stage premenopausal ER+BC patients with reduced survival outcomes.

Results

Exhausted CD8⁺ T Cells are Enriched in Subsets of Breast Cancer Patient Tumors

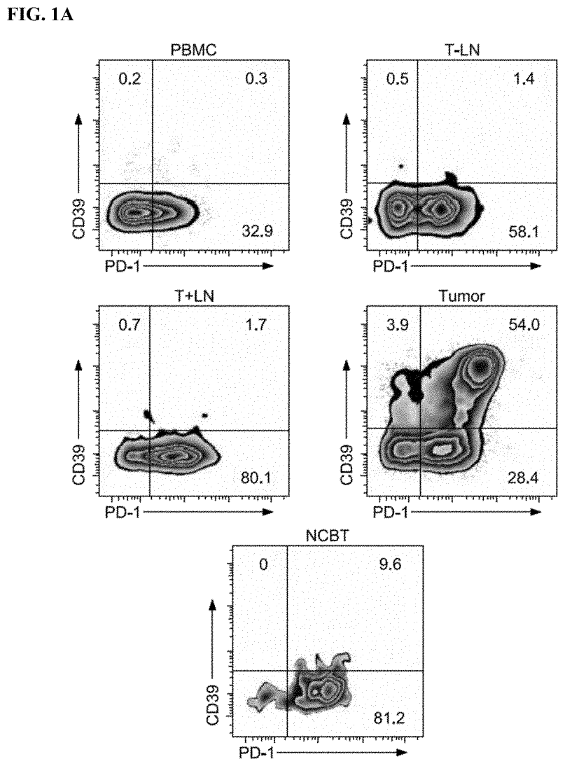

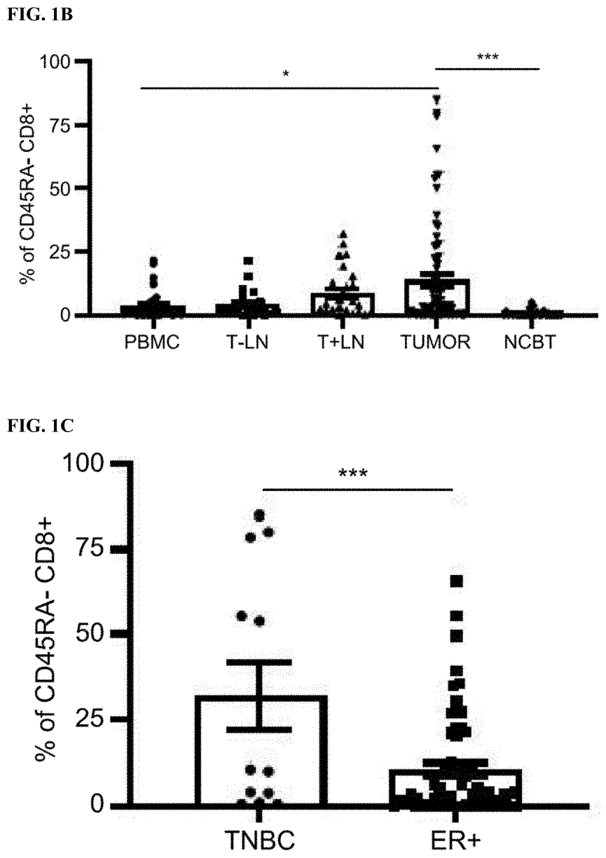

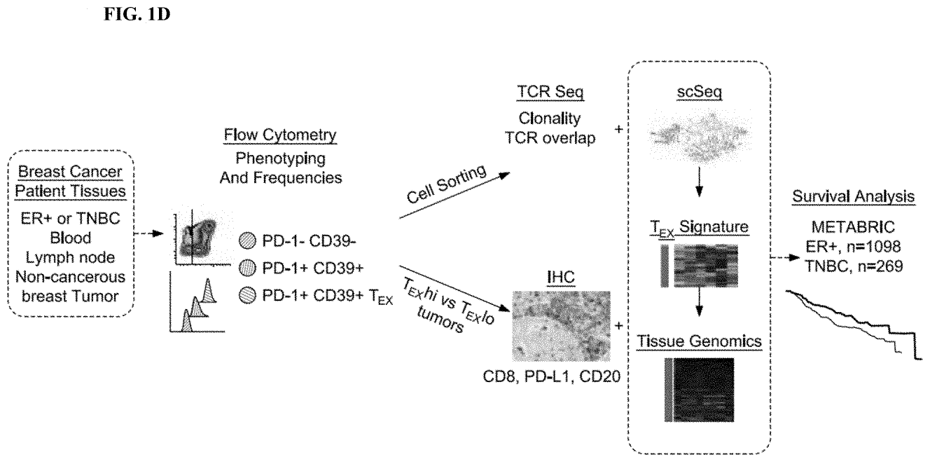

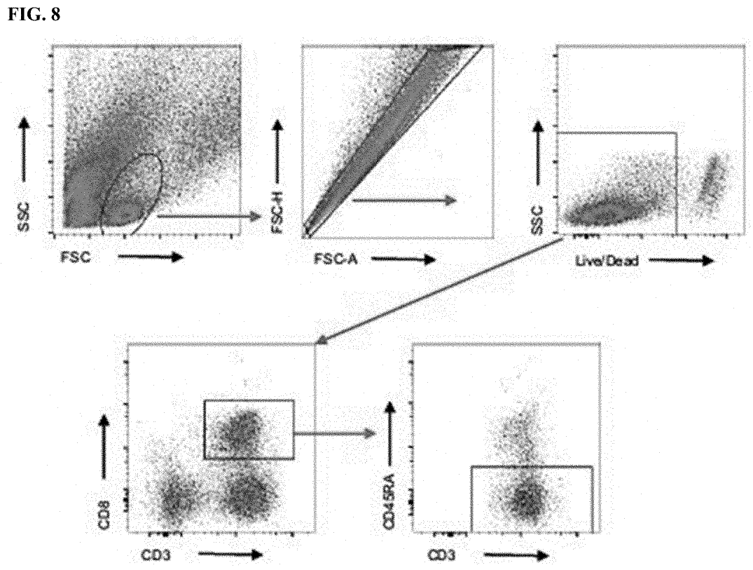

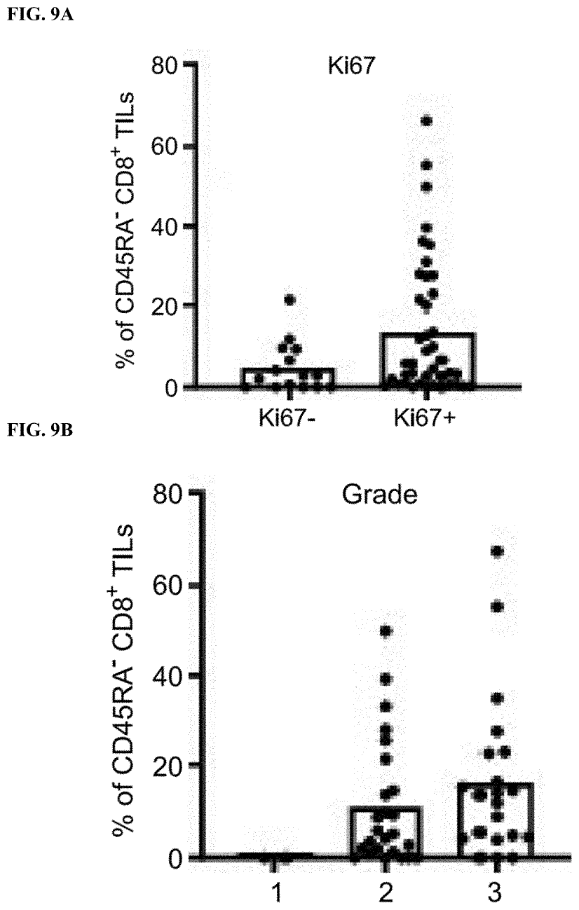

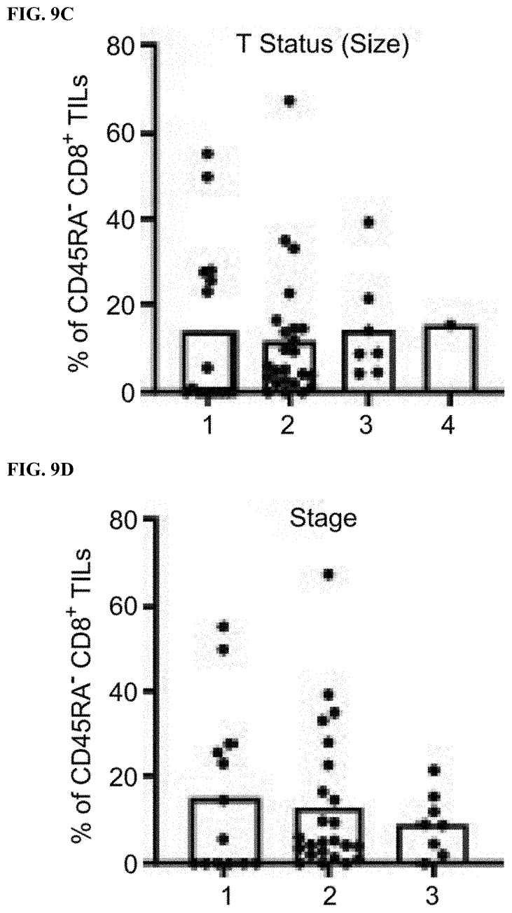

We examined BC patient peripheral blood mononuclear cells (PBMCs), tumor negative tumor draining lymph nodes (T− LNs), tumor positive tumor draining lymph nodes (T+ LNs), primary tumors, and non-cancerous breast tissue (NCBT) by flow cytometry for the presence of CD8⁺ T cells expressing T cell exhaustion markers PD-1 and CD39 (FIG. 1A; gating strategy FIG. 8). Amongst antigen experienced (CD45RA⁻) CD8⁺ T cells, PD-1⁺ cells were common in all tissues, but frequencies of PD-1⁺ CD39⁺ CD8⁺ T cells were highest in primary tumors, followed by T+ LNs (FIG. 1B). PD-1⁺ CD39⁺ CD8⁺ T cells were rarely detected in PBMCs and never in NCBT. Notably, T+ LNs and T− LNs displayed no significant differences in frequencies of PD-1⁺ CD39⁺ CD8⁺ T cells. We observed high variability in the frequency of PD-1⁺ CD39⁺ within CD8⁺ TTLs and a higher frequency on average in TNBC tumors than ER+ tumors (FIG. 1C). Overall, the frequency of PD-1⁺ CD39⁺ CD8⁺ TTLs did not correlate with Ki-67 status, tumor size (pathological T status), or patient stage (FIG. 9). Higher grade ER+ tumors tended to have increased frequencies of PD-1⁺ CD39⁺ CD8⁺ TILs as compared to lower grade ER+ tumors, but this observation lacked statistical significance due to high interpatient variability.

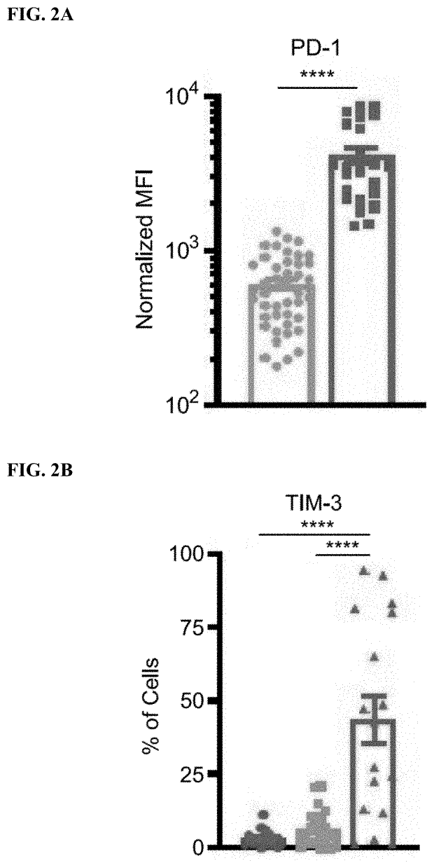

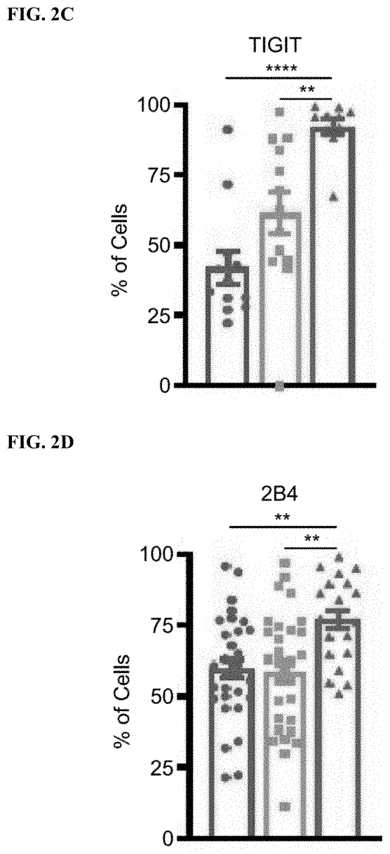

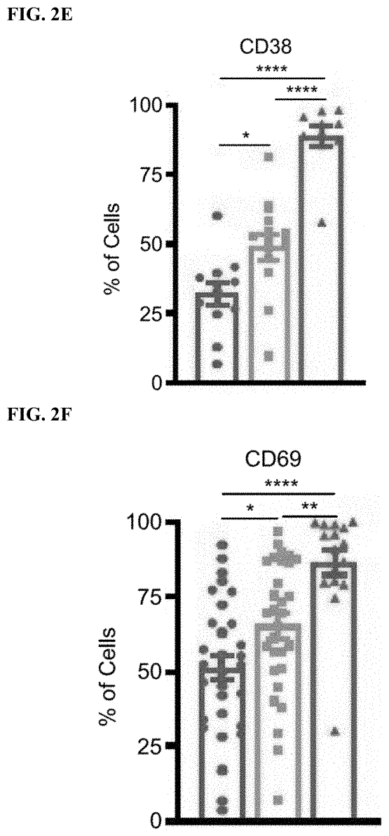

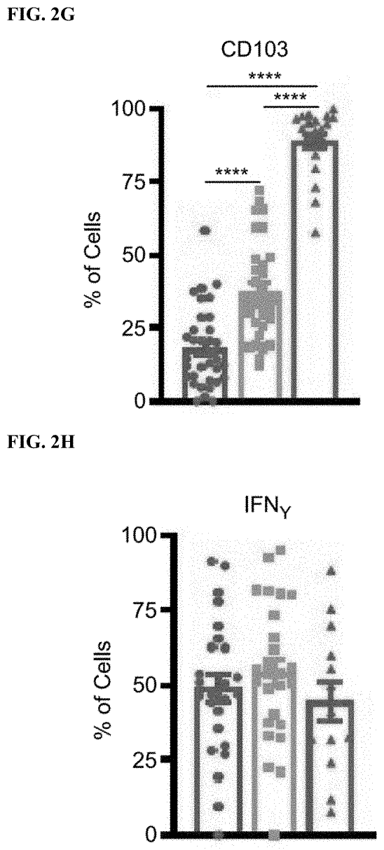

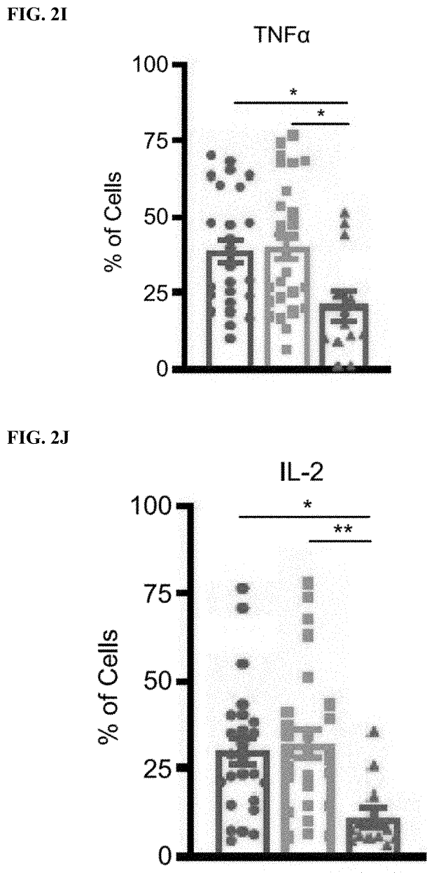

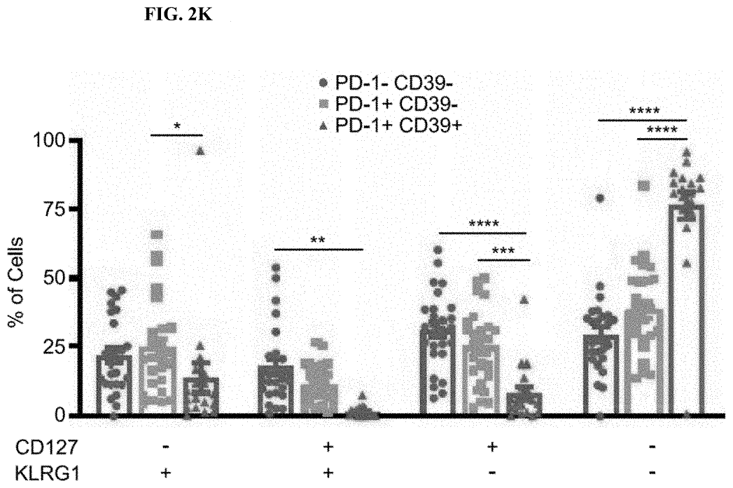

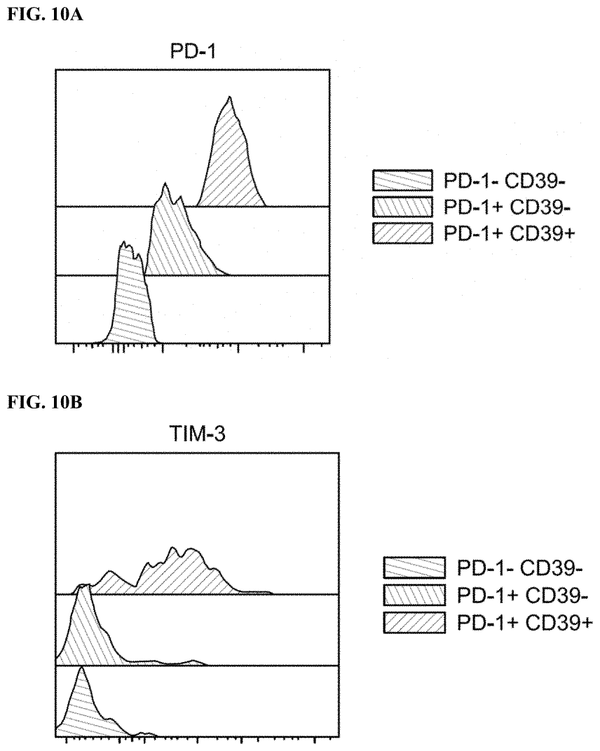

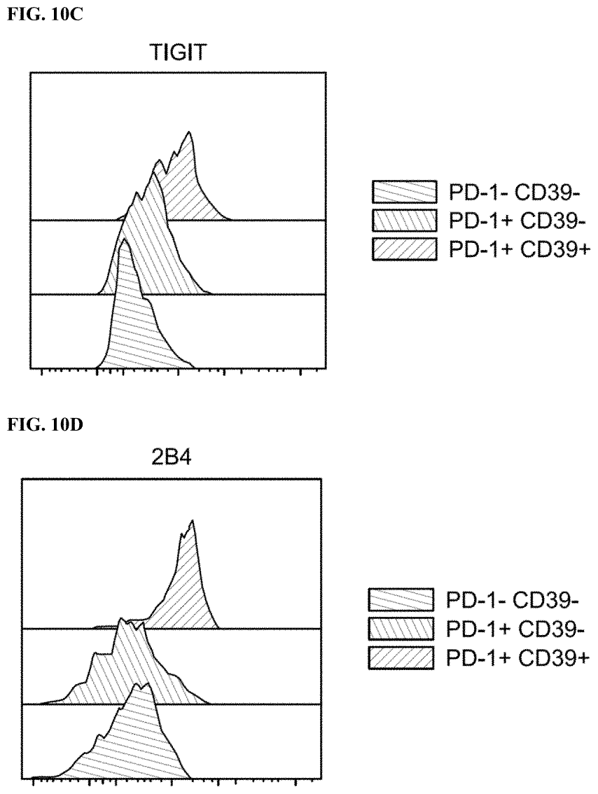

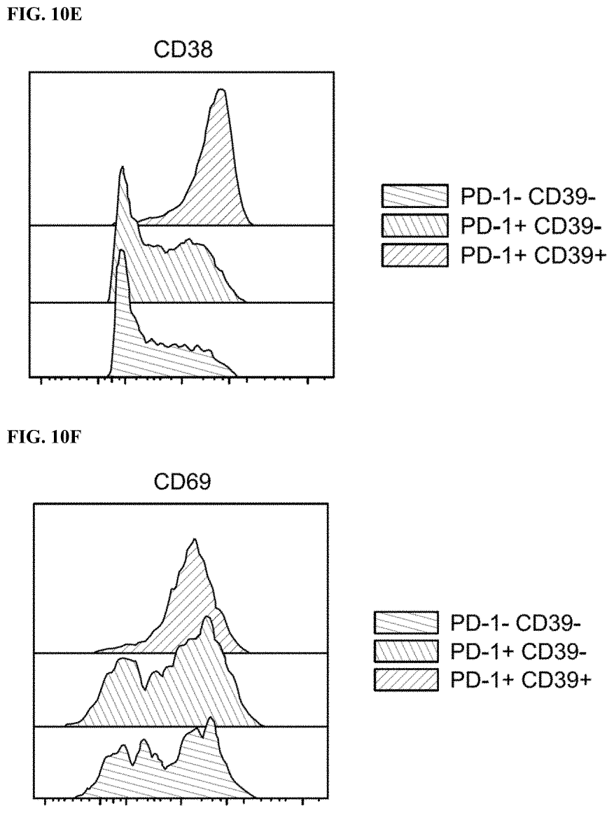

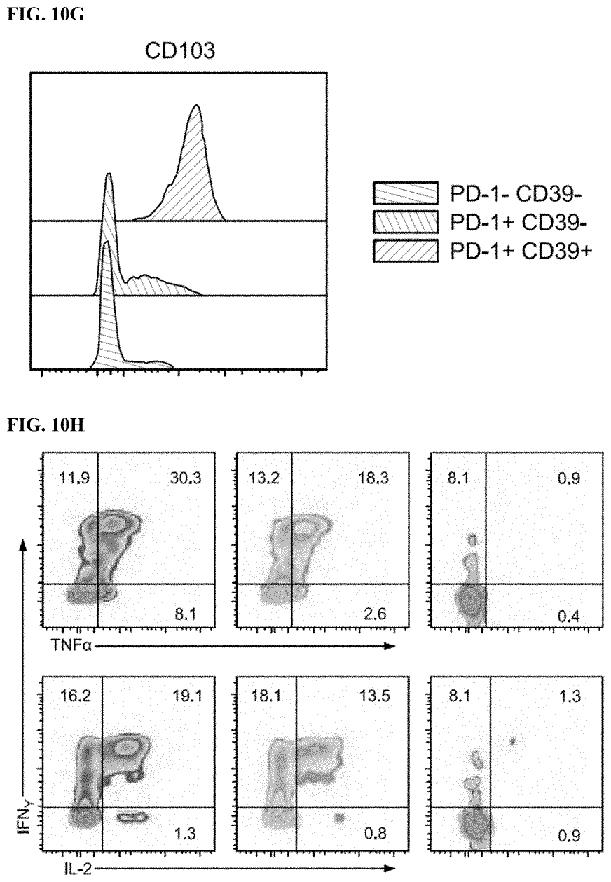

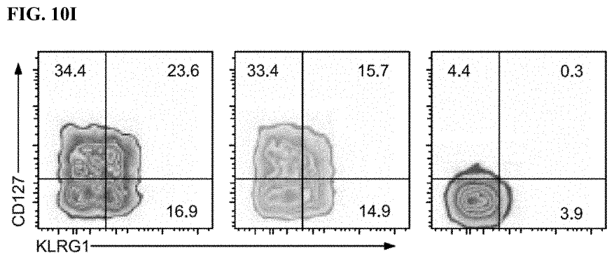

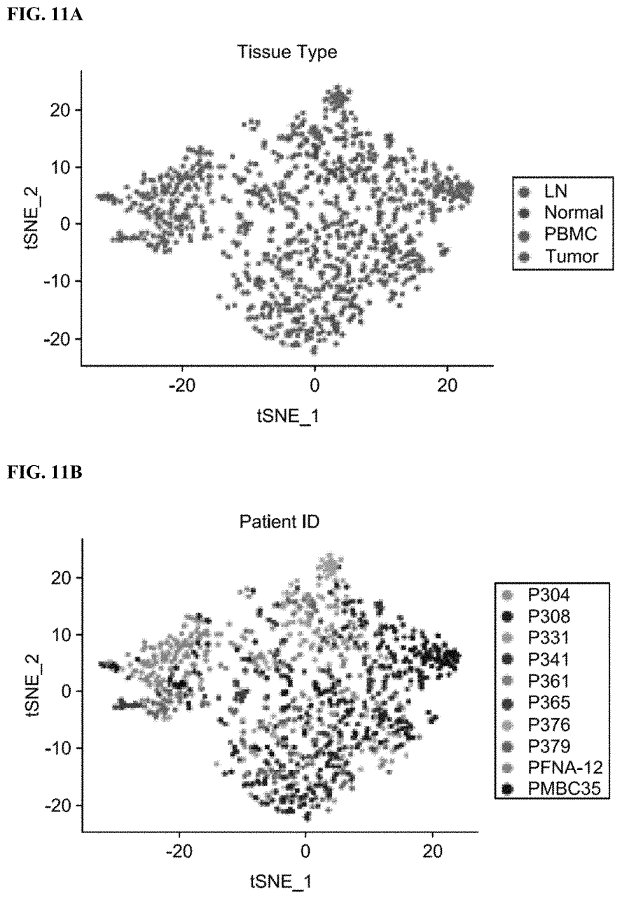

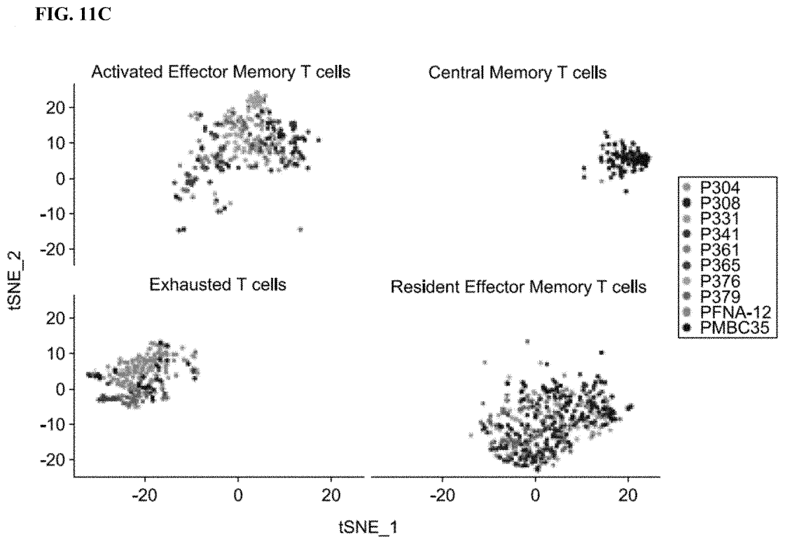

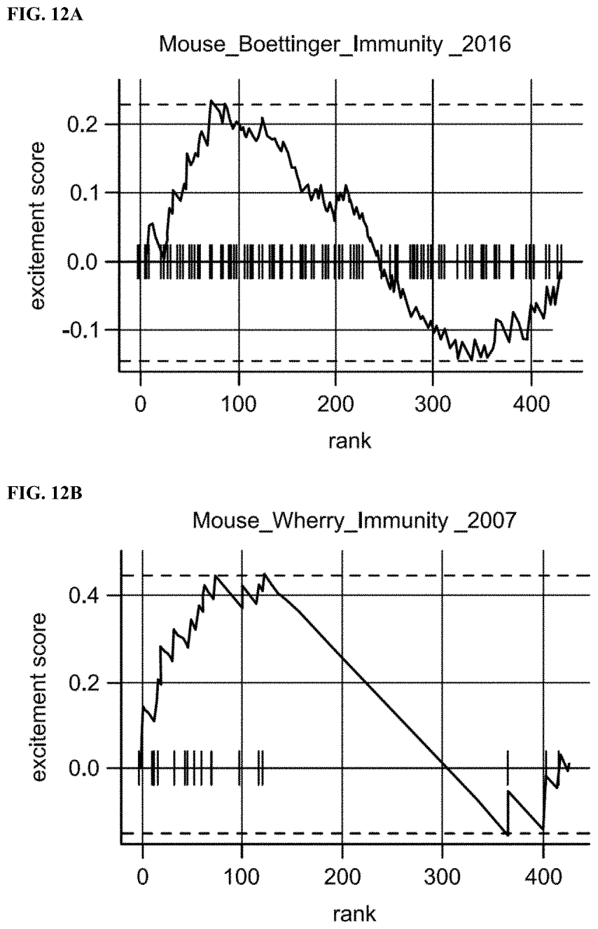

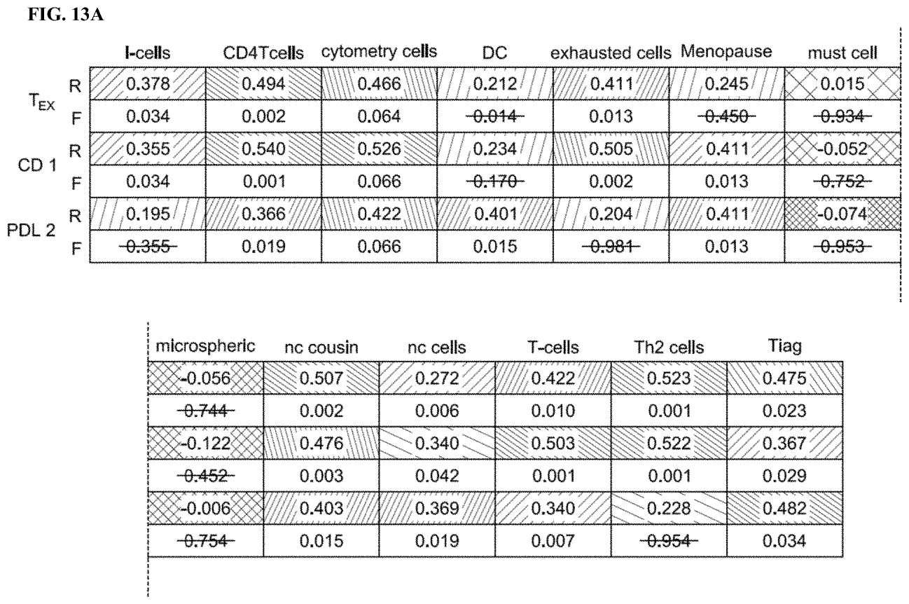

We next set to elucidate the relationship between PD-1⁺ CD39⁺ CD8⁺ TILs, TME features, and patient survival using a multi-omics approach (FIG. 1D). Further characterization of BC patient PD-1⁺ CD39⁺ CD8⁺ TILs protein expression was performed by flow cytometry to determine if they met canonical definitions of T cell exhaustion. PD-1 levels were significantly higher on PD-1⁺ CD39⁺ CD8⁺ TILs relative to PD-1⁺ CD39⁻ CD8⁺ TILs (FIG. 2A; FIG. 10A), identifying them as PD-1 ‘high’ CD8⁺ T cells described in other tumor type (23). Relative to other CD8⁺ TILs, higher percentages of PD-1⁺ CD39⁺ CD8⁺ TILs expressed molecules TIM-3, TIGIT, 2B4, and CD38 (FIGS. 2B-2E, FIGS. 10B-10E). Similarly, higher percentages of PD-1⁺ CD39⁺ CD8⁺ TILs expressed resident memory markers CD69 and CD103 as compared to other CD8⁺ TILs (FIGS. 2F-2G; FIGS. 10F-10G).

We then confirmed PD-1⁺ CD39⁺ CD8⁺ TILs as functionally exhausted compared to other CD8⁺ TTLs by examining their capacity to produce effector cytokines IFNγ, TNFα, and IL-2 (FIGS. 2H-2J; FIG. 10H). PD-1⁺ CD39⁻ and PD-1⁻ CD39⁻ CD8⁺ TILs displayed no differences in IFNγ, TNFα, or IL-2 production capacity, highlighting our previous findings that PD-1 expression alone does not identify an exhausted phenotype (18). In contrast, PD-1⁺ CD39⁺ CD8⁺ TILs demonstrated significant loss in production capacity of both TNFα and IL-2, while mostly retaining IFNγ production capacity. Such functional data formally identifies PD-1⁺ CD39⁺ CD8⁺ TILs in human breast tumors as CD8⁺ T_{EX} with similar functional and phenotypic profiles of T_{EX} described by others in the context of other cancer malignancies and chronic disease settings (16, 17, 22, 24).

Next, we examined expression of proteins CD127 (IL-7Rα) and KLRG1 to assess PD-1⁺ CD39⁺ CD8⁺ TILs for evidence of terminal differentiation (FIG. 2K; FIG. 10I). CD127 expression is critical for homeostatic proliferation and maintenance of memory T cells, while KLRG1 expression signifies an effector T cell status (25). Loss of both CD127 and KLRG1 has been associated with a severe T cell exhaustion phenotype (26, 27). PD-1⁺ CD39⁺ CD8⁺ TILs primarily displayed a CD127⁻ KLRG1⁻ phenotype. Comparatively, both PD-1⁺ CD39⁻ and PD-1⁻ CD39⁻ CD8⁺ TILs contained cell populations with mixed expression of CD127 and KLRG1. Taken together this phenotyping illustrates PD-1⁺ CD39⁺ CD8⁺ TILs found in BC tumors as highly activated cells with both exhausted and tissue residency characteristics.

Exhausted CD8⁺ T Cells in Human Breast Tumors are Transcriptionally Distinct

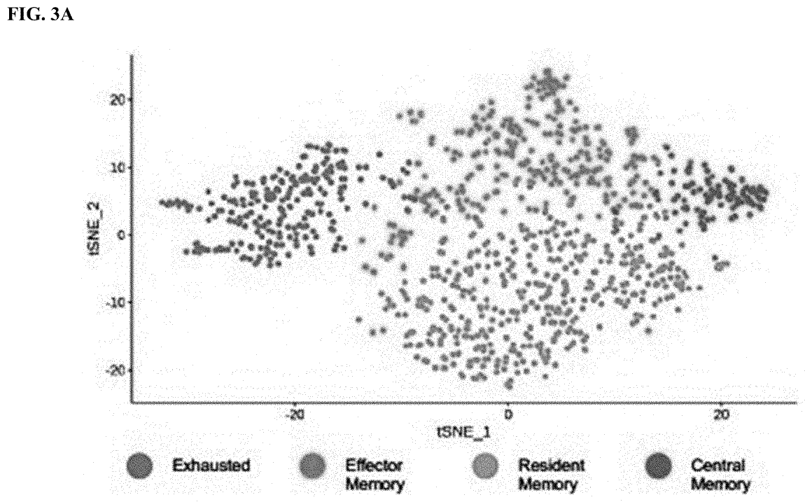

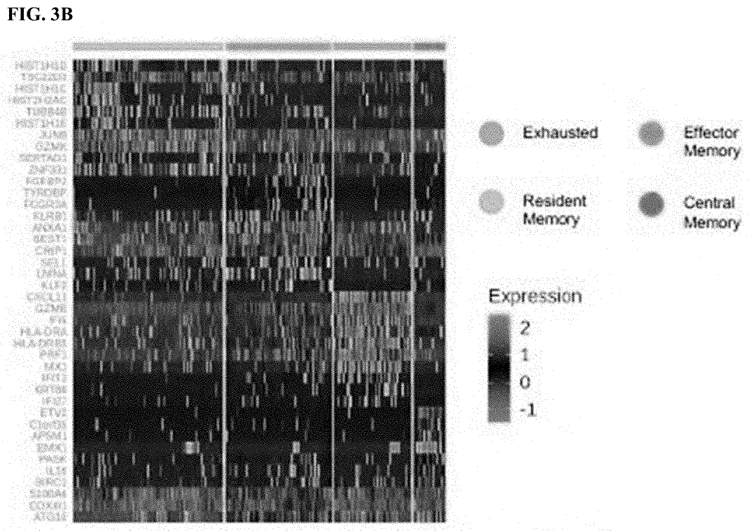

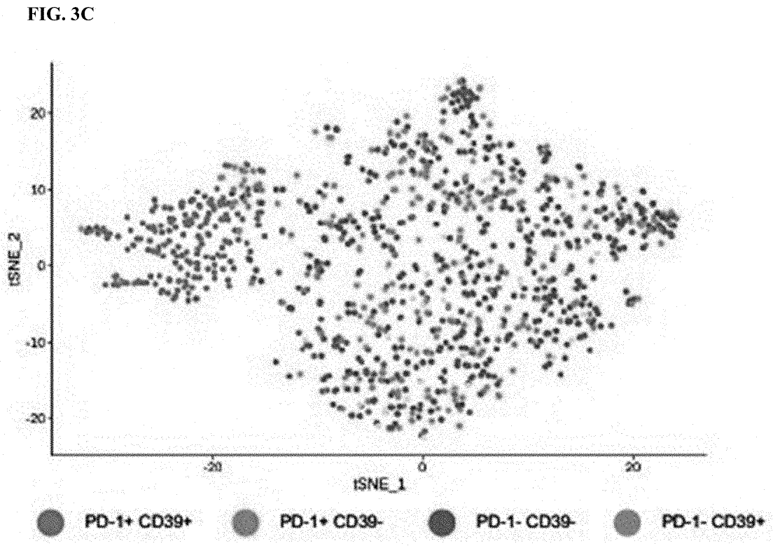

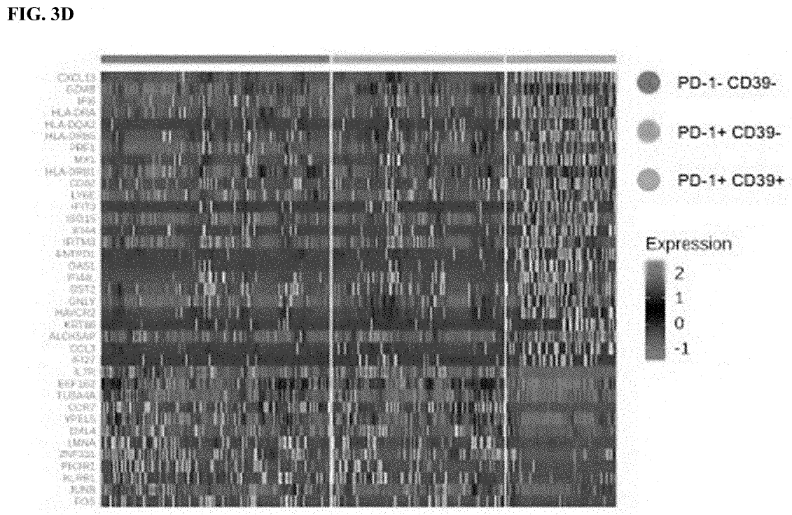

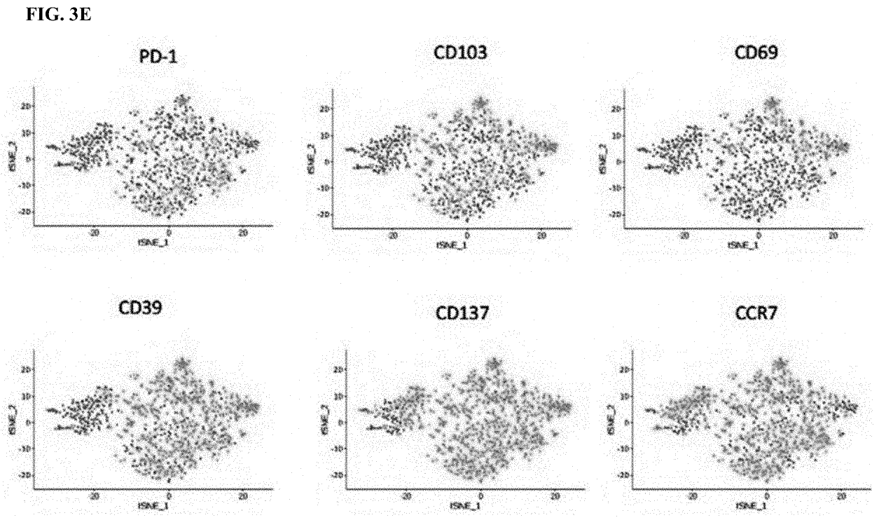

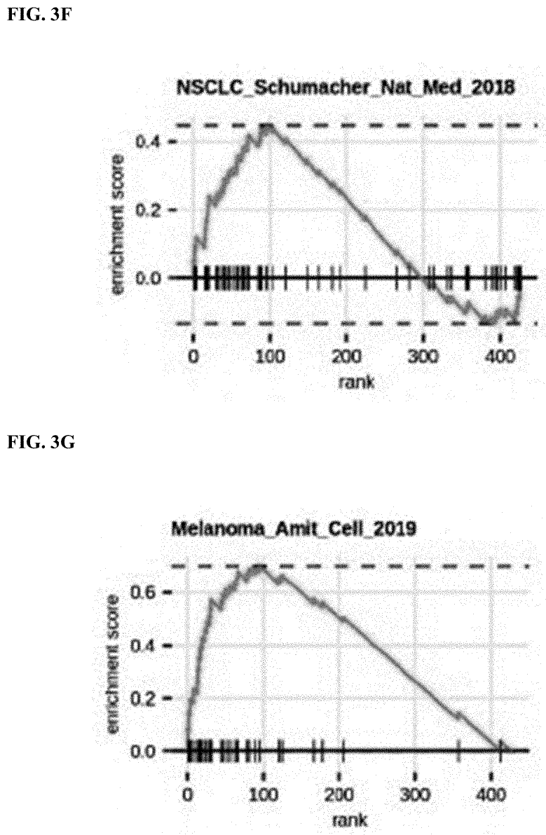

CD8⁺ T cell exhaustion has been demonstrated as a transcriptionally and epigenetically discrete functional state in various disease settings (28-31). To assess this in the context of BC we employed single cell RNA sequencing of patient CD8⁺ T cells from 10 different BC patients, including 9 primary tumors, 2 T+LNs, 3 NCBTs, and 7 matched PBMCs samples (FIG. 11). CD8+ T cells stained for PD-1, CD39, CD103, CD69, CD137, and CCR7 were single cell index sorted for downstream whole transcriptome analysis. Unbiased Seurat cluster analysis found CD8⁺ T cells to be composed of four major clusters with discrete gene expression patterns (FIGS. 3A-3B). As expected PD-1⁺ CD39⁺ T cells occupied a unique cluster, while surprisingly PD-1⁺ CD39⁻ and PD-1⁻ CD39⁻ were indiscriminately found in all other T cell clusters and showed no major differences in gene expression (FIGS. 3C-3D).

Cell surface protein expression information to annotate the four CD8⁺ T cell clusters as exhausted T cells (PD-1⁺ CD39⁺), resident effector memory T cells (CD39⁻, CD103⁺, CD69⁺), effector memory T cells (CD39⁻, CD103^{+/-}, CD69^{+/-}), and central memory T cells (CCR7) using index sort collected data (FIG. 3E). CD137 was found almost exclusively in the exhausted T cell cluster. CD103 and CD69 expression across several clusters indicates the acquisition of a CD103 CD69⁺ phenotype as T cells transition through these phenotypes. Unsurprisingly, the CCR7 expressing central memory T cell cluster was largely identified in PBMC derived T cells.

Analysis of gene expression differences between clusters revealed several key differences between T cell populations. The central memory T cell cluster, mostly of PBMC origin, expressed genes related to proliferation capacity (PASK, IL16) and anti-apoptosis (BIRC2), along with the memory T cell associated gene S100A6(32). The activated effector memory T cell cluster expressed genes linked to T cell activation (LMNA, ANXA1), effector function (FGFBP2, KLRB1), and T cell trafficking (SELL, KLF2)(33, 34). Intriguingly, the resident effector memory T cluster demonstrated highly differentiated upregulation of histone genes and regulatory elements (SERTAD1, ZNF331) that may play a role in cell cycle regulation. The resident effector memory T cluster also displayed upregulated GZMK, in contrast with GZMB and PRF1 upregulation in the exhausted T cell cluster. This observed T cell subset specific granzyme utilization likely reflects T cell differentiation stage specific changes in granzyme expression profiles observed by others (35). Finally, the exhausted T cell cluster showed transcriptional upregulation of genes associated with increased cytolytic activation (GZMB, PRF1), T cell activation (HLA-DRA), interferon response elements (IFI6, MX1, IFI27), and the B cell chemoattractant CXCL13. Additionally, we observed downregulation of activator protein 1 (AP-1) complex molecules JUNB and FOS; downregulation of these genes has been shown by others to mark chronically activated T_{EX} (36, 37).

We next generated a T_{EX} gene signature composed of genes significantly elevated in T_{EX} relative to other CD8⁺ T cell populations. Log 2 fold changes of genetic markers were considered as the weights in the signature. Our T_{EX} gene signature contains 25 genes, including CXCL13, GZMB, IFI6, HLA-DRA, HLA-DQA2, HLA-DRB5, PRF1, and MX1. The full gene signature and relative gene fold changes are shown in FIG. 22. We then compared our T_{EX} gene expression signature to T_{EX} gene signatures produced by other groups using gene set enrichment analysis (GSEA). We found that our T_{EX} signature shared similarities to those identified from lung cancer and melanoma TILs (FIGS. 3F-3G) (24, 38). We also verified that our T_{EX} gene signature had overlap to those produced in LCMV murine models of T cell exhaustion (FIG. 12) (37, 39). Thus, the transcriptional signature of T_{EX} in BC patients shared common features to both those seen in other disease states and classically defined exhausted T cells. Together our single cell data confirms CD8⁺ T_{EX} as an activated, transcriptionally distinct, CD8⁺ TIL population that has likely clonally expanded in response to cognate tumor antigens.

Increased CD8⁺ T_{EX} Associates with IFNγ Signature Rich and Immunologically Distinct Tumors

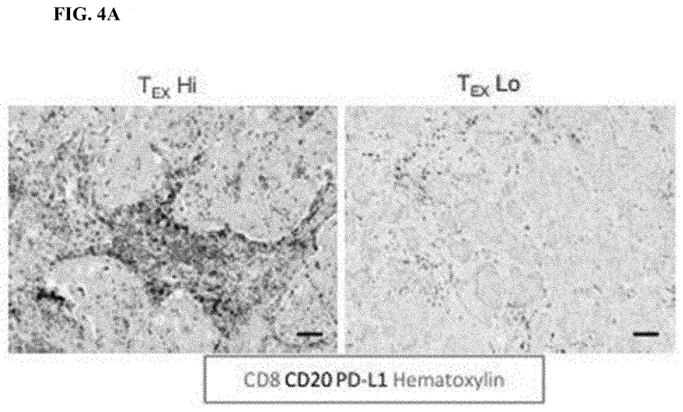

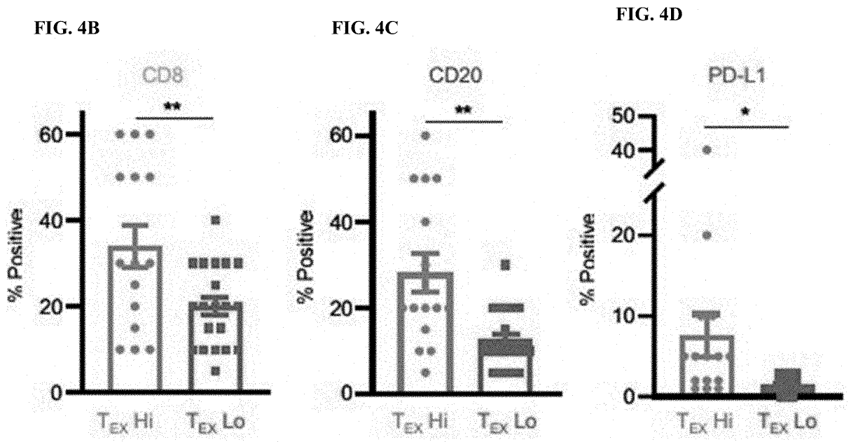

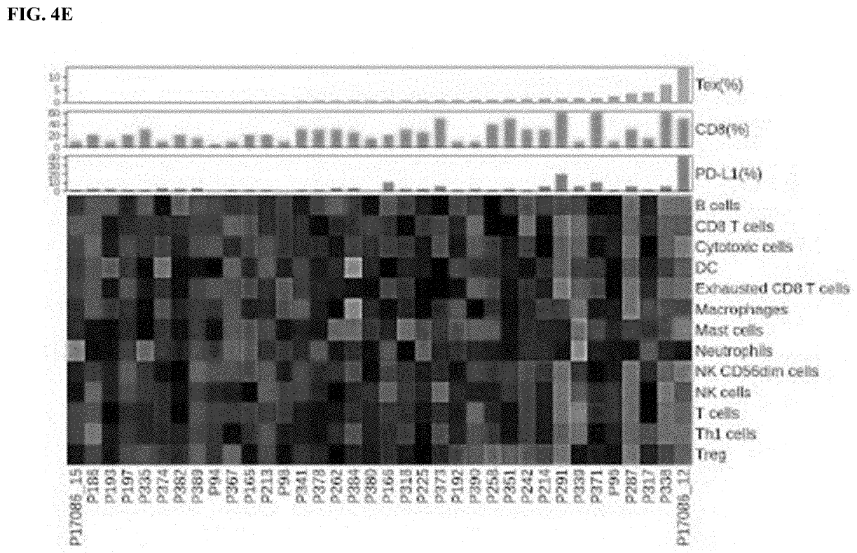

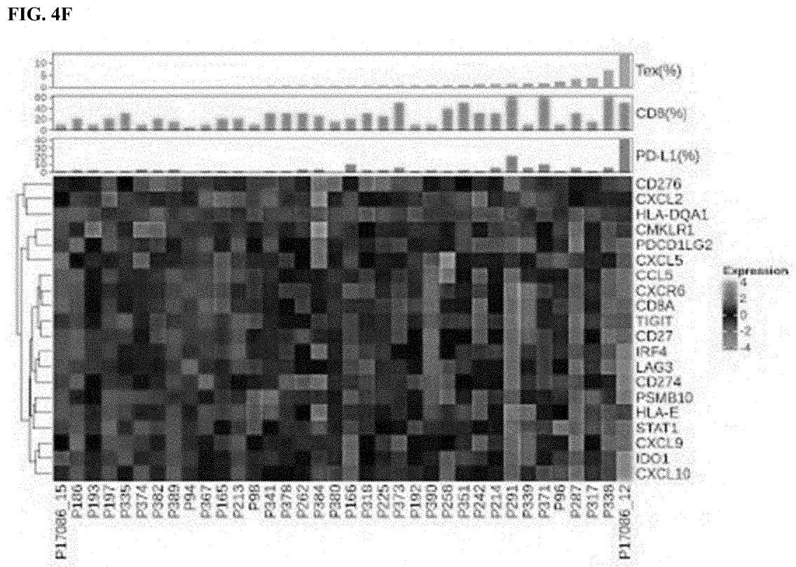

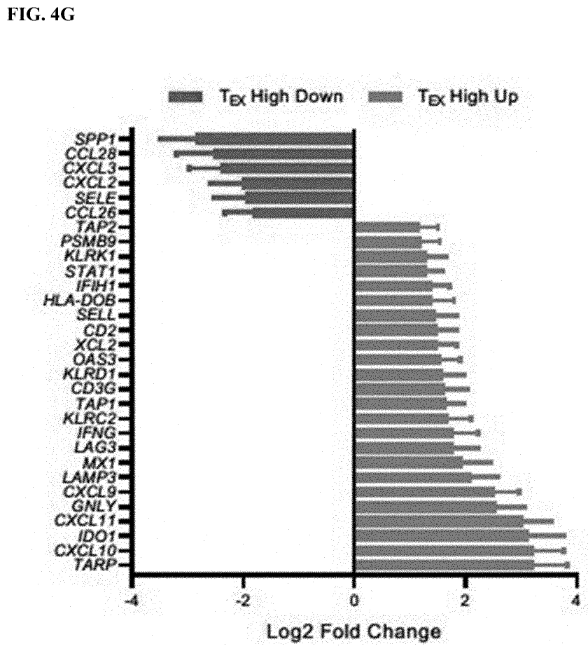

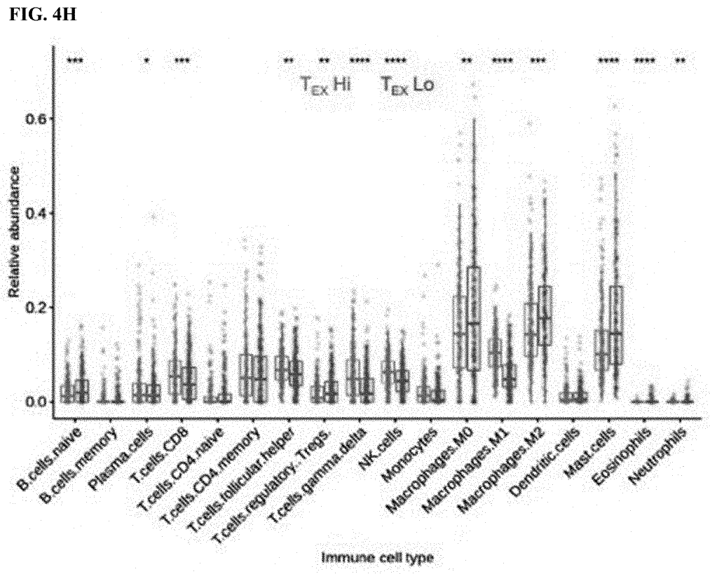

Given that CD8⁺ T_{EX} identified in BC patient tumors displayed a highly activated phenotype and largely retained the capacity to produce IFNγ, we next asked how their presence correlated with differences in TME features. ER+ tumors with known fractions of CD8⁺ T_{EX} as identified by flow cytometry were curated into T_{EX} ‘high’ (T_{EX}hi) and T_{EX} ‘low’ (T_{EX}lo)tumors as defined by above and below the overall median for % T_{EX} of CD8⁺ TILs (8%). Pathologist assessment of CD8, CD20, and PD-L1 expression was then performed on these tumors with immunohistochemistry stained slides (FIG. 4A). CD8⁺ T cell infiltration was higher in several, but not all, T_{EX}hi tumors (FIG. 4B). In contrast, CD20⁺ B cell infiltration was higher in the majority of T_{EX}hi tumors (FIG. 4C). Although PD-L1 expression was found to be highly variable in samples, strikingly, all tumors with stroma scoring PD-L1+ of 5% or higher were found in T_{EX}hi tumors (FIG. 4D).

ER+ tumors with known CD8⁺ T_{EX} abundance from flow cytometry were assessed with the Nanostring PanCancer Immune profiling panel for immune cell composition and differential gene expression. CD8⁺ T_{EX} in tumors correlated with higher abundance of a variety of immune subsets including B cells, overall CD8⁺ T cells, ‘exhausted CD8⁺ T cells’, Th1 cells, Tregs, and CD56^{dim} NK cells as identified by standard Nanostring signatures (FIG. 4E). Given the association between activated T cells, IFNγ production, and PD-L1 expression in the TME, we assessed the expression of the previously reported ‘tumor inflammation signature,’ which is mainly composed of IFNγ regulated genes (40). Several of these genes were significantly correlated with the presence of CD8⁺ T_{EX}, including CXCL10, IDO1, CXCL9, STAT1, CD274 (PD-L1), and LAG3 (FIG. 4F). Interestingly, CD276 and CXCL2 expression were not positively correlated with CD8⁺ T_{EX}. Other genes significantly upregulated in T_{EX}hi tumors included IFNG itself, TARP, GNLY, MX1, and TAP2. Notably, genes SPP1, CCL28, CXCL3, SELE, and CCL26 were all decreased in T_{EX}hi tumors (FIG. 4G).

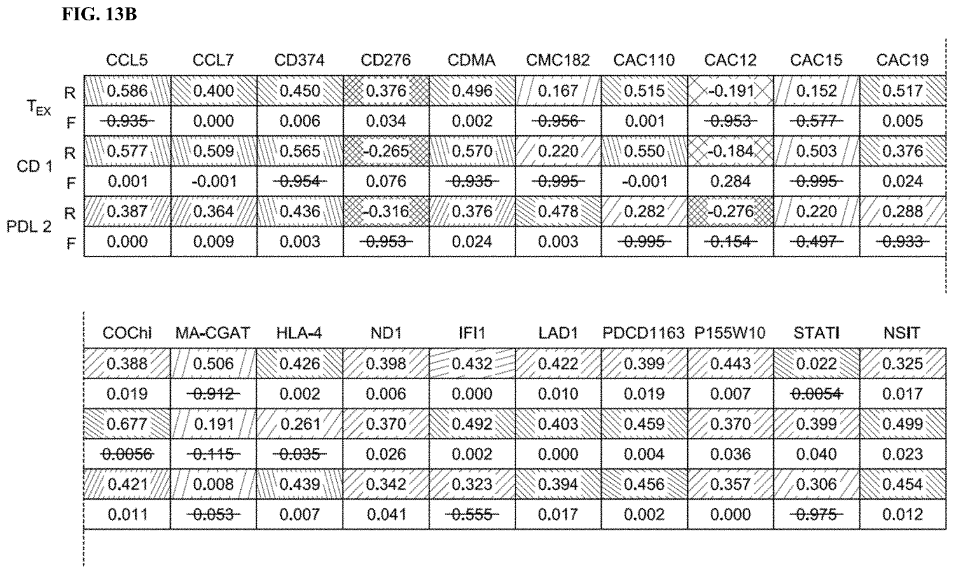

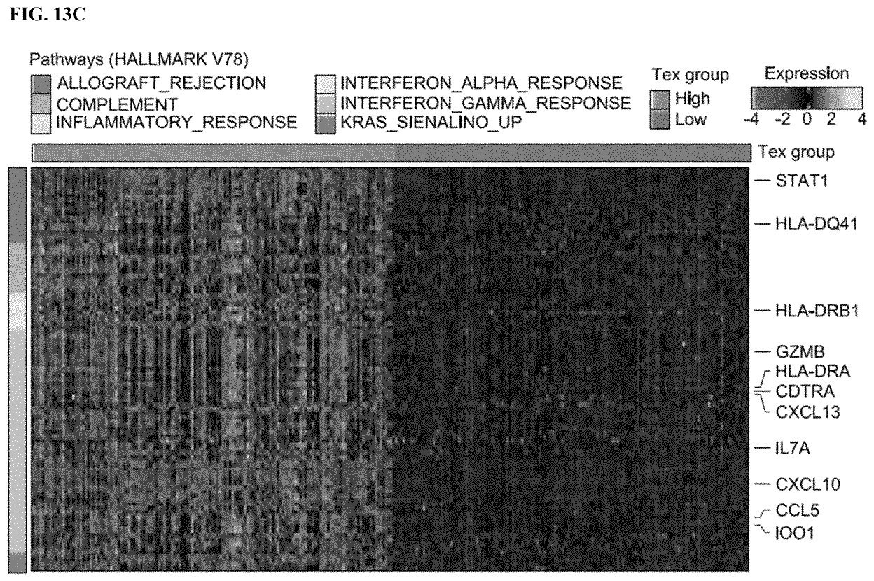

To expand on our tissue-based observations of T_{EX}hi and T_{EX}lo ER+ tumors, we turned to the Molecular Taxonomy of Breast Cancer International Consortium (METABRIC) public data source for gene expression analysis in a larger cohort of BC tumors (41). We designated ER+ tumors as T_{EX}hi (top 25%) and T_{EX}lo (bottom 25%) based on expression of our single cell sequencing derived T_{EX} gene signature and performed differential gene expression analysis. T_{EX}hi tumors demonstrated significantly increased expression level of numerous genes involved in immune surveillance and activation marked by increased expression level of allograft rejection, inflammatory response, and interferon response Hallmark pathways (FIG. 13). In concurrence with Nanostring analysis of our own tumor samples, these included IFNγ signaling genes STAT1, CXCL10, and IDO1, antigen presentation molecules HLA-DQA1 and HLA-DRB1, important T cell molecules GZMB and IL7R, and B cell related molecules CD79A and CXCL13. Next, we utilized CIBERSORTvX to interrogate differences in immune composition between T_{EX}hi and T_{EX}lo tumors by assessing relative abundance of various immune populations (42). Notably, T_{EX}hi tumors were composed of higher fractions of M1 macrophages, NK cells, 76 T cells, and CD8⁺ T cells (FIG. 4H). In comparison T_{EX}lo tumors were composed of higher fractions of M2 macrophages, M0 macrophages, mast cells and naïve B cells. In summary, T_{EX}hi tumors display an ‘inflamed tumor’ phenotype, with upregulation of numerous IFNγ associated genes, increased chemokines, antigen presentation related molecules, and anti-tumor immune subsets such as M1 macrophages, NK cells, and effector CD8⁺ T cells.

T_{EX} Signatures Denote Prognostic Outcome in Breast Cancer Patients

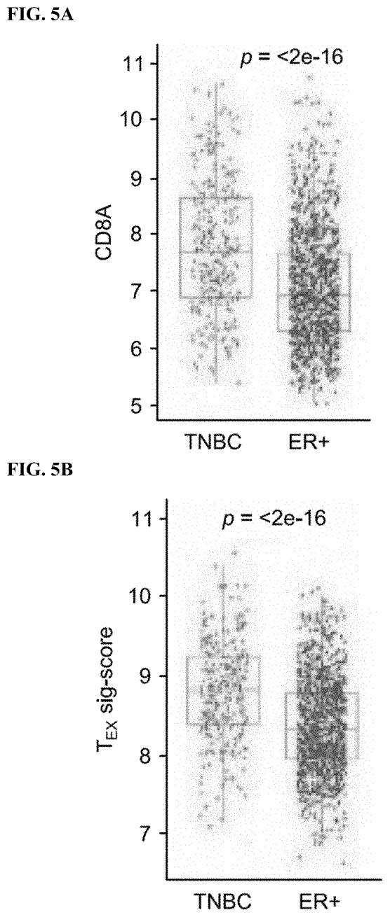

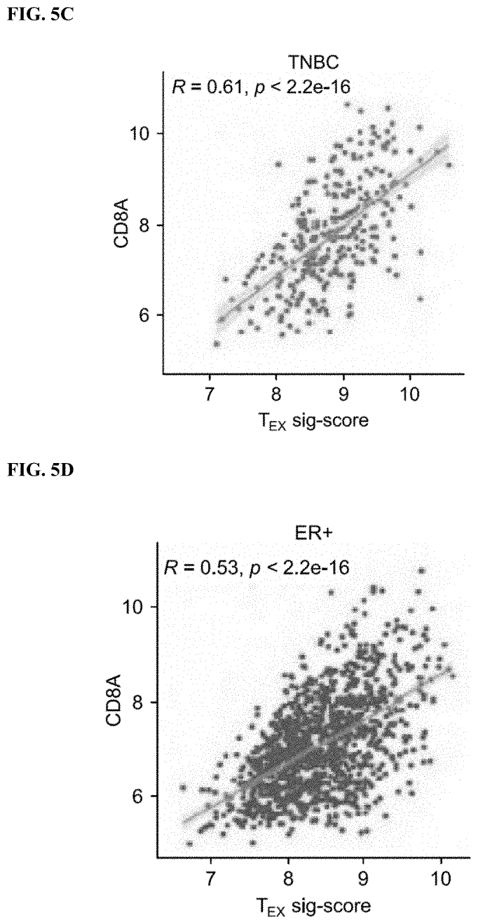

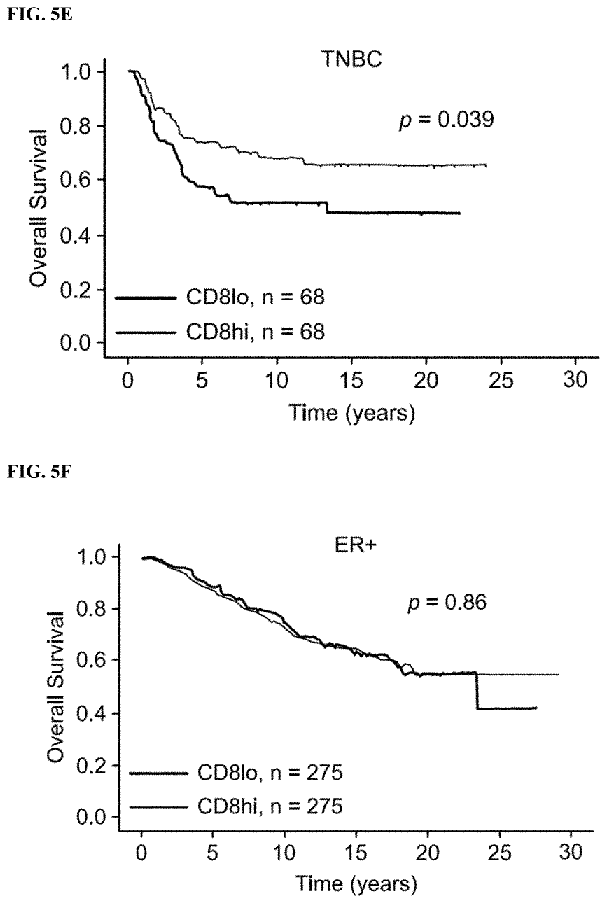

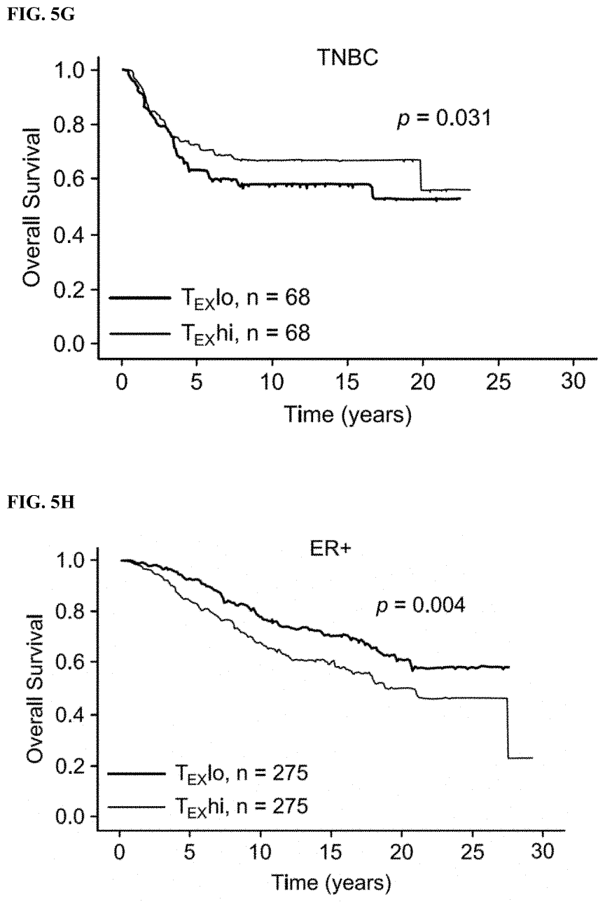

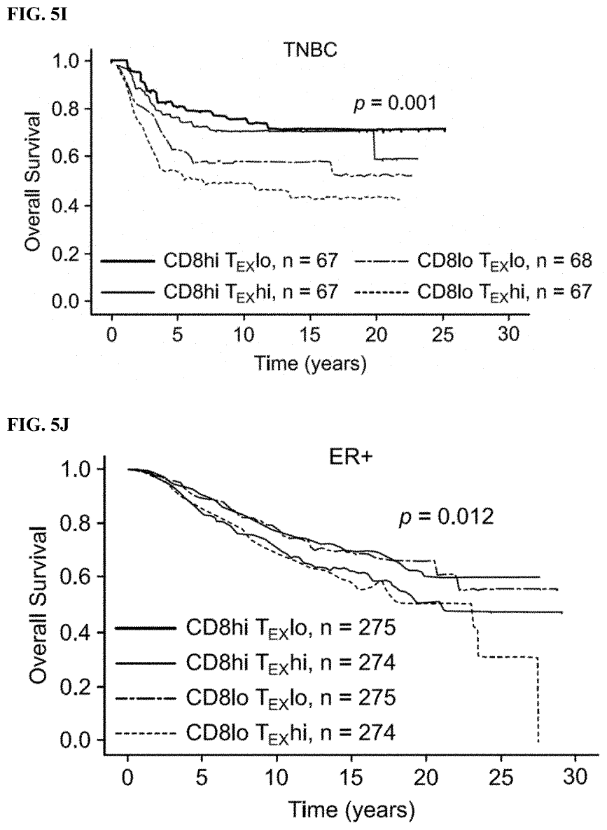

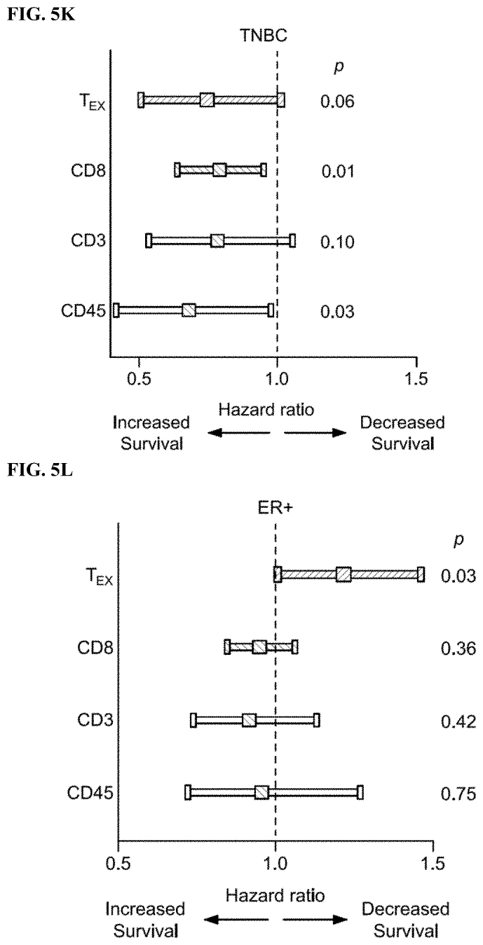

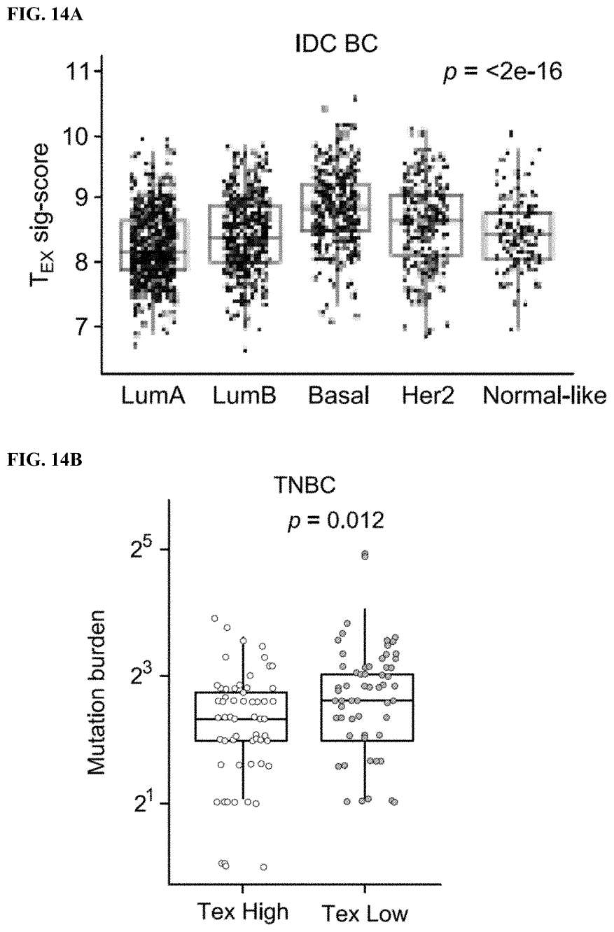

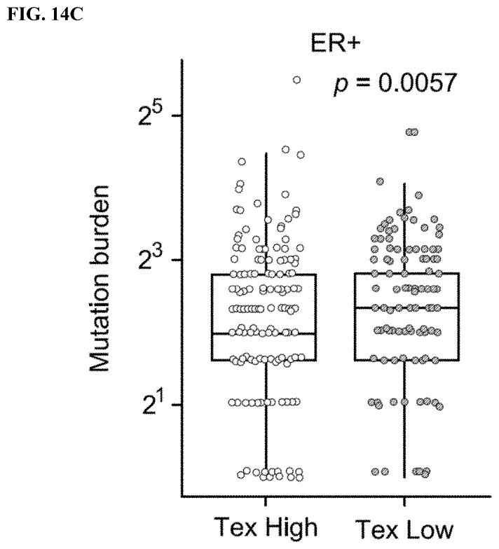

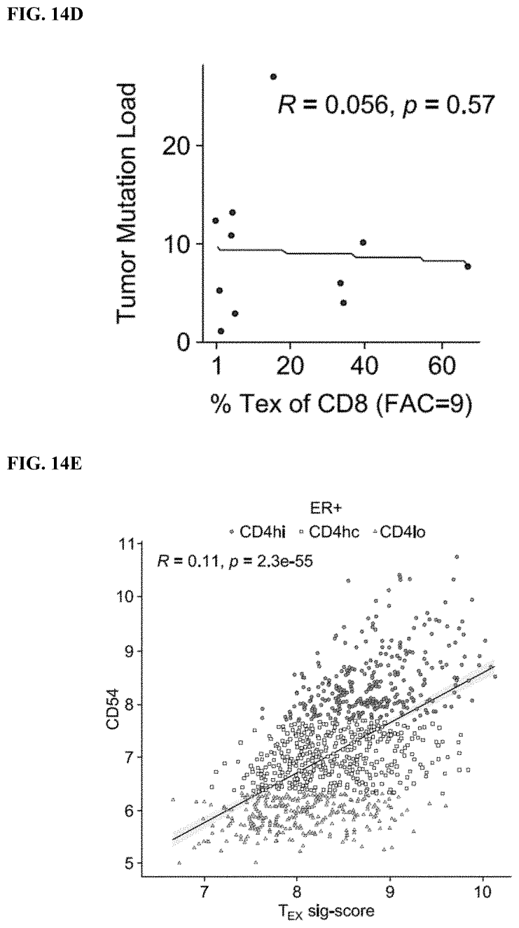

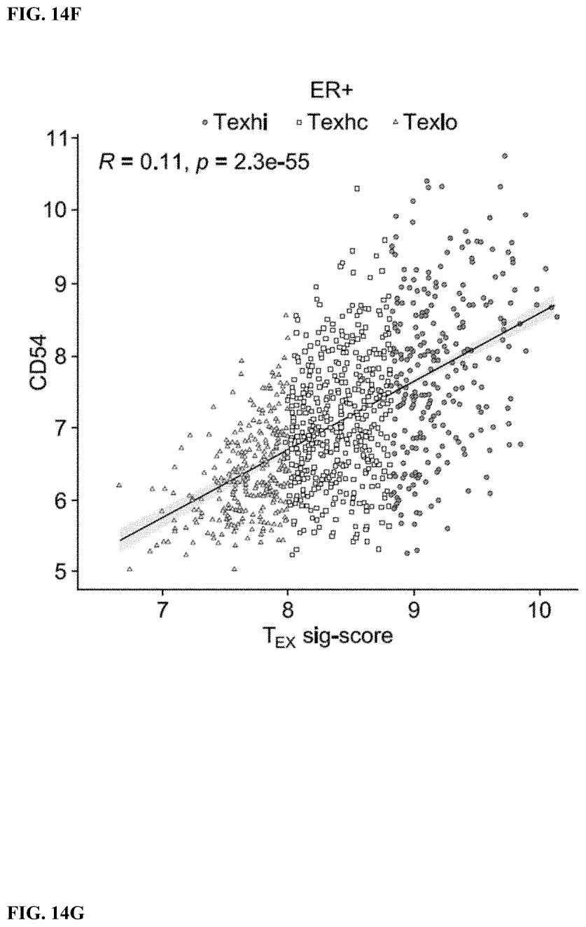

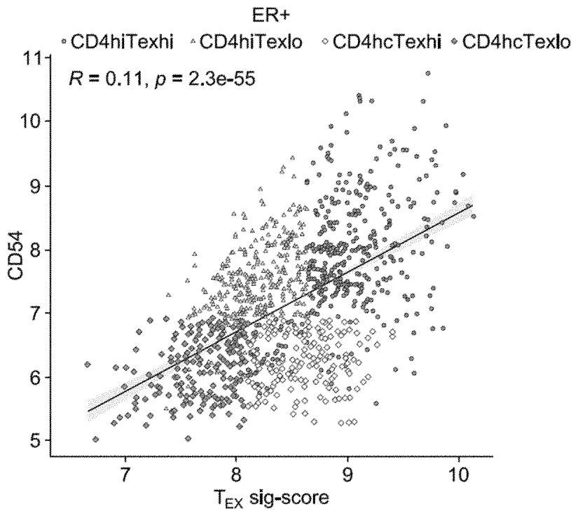

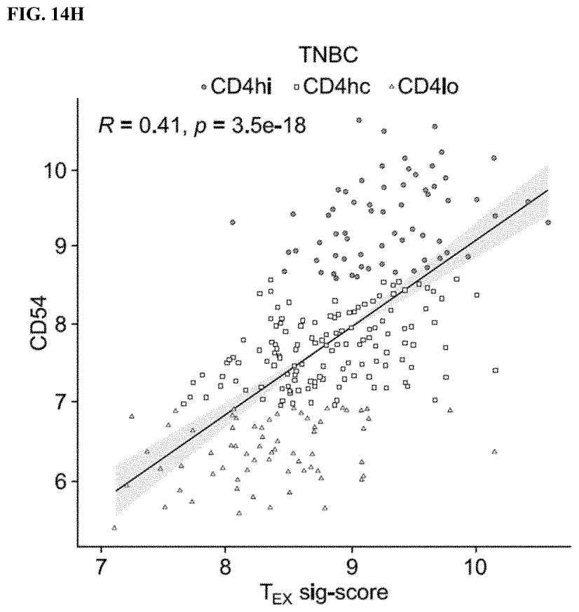

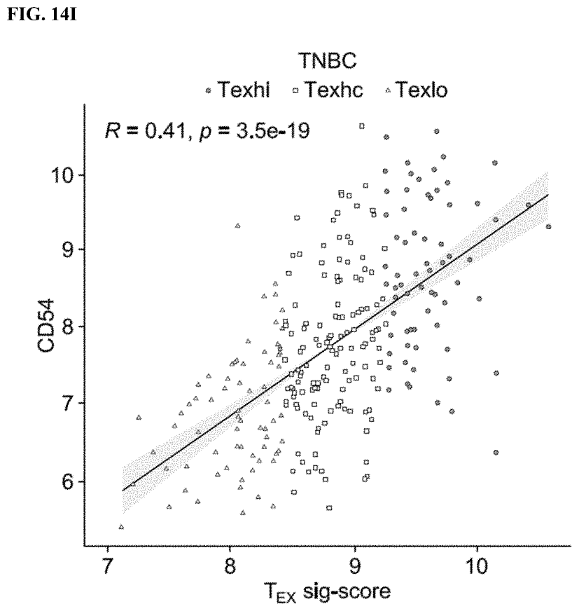

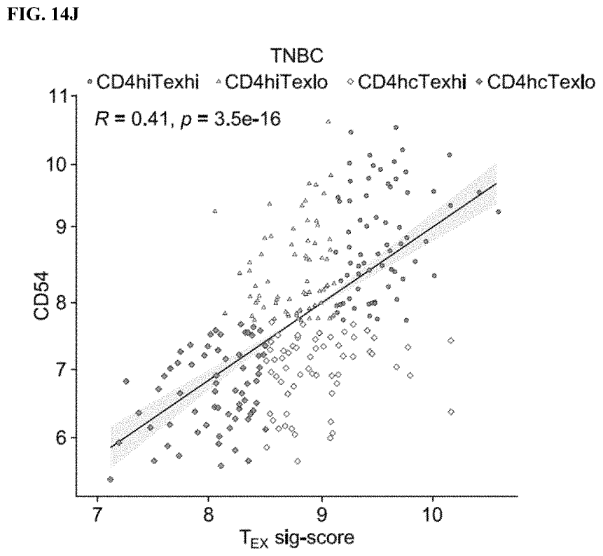

We next aimed to unravel the relationship between CD8⁺ T_{EX}, BC tumor characteristics, and patient outcomes within the METABRIC dataset. As expected, increased expression level of CD8A was found in TNBC tumors compared to ER+ tumors (FIG. 5A). We next found T_{EX} signatures scores were higher in TNBC tumors as compared to ER+ tumors (FIG. 5B), as also observed in our flow cytometry data. In line with these observations, PAM50 molecular classification of tumors demonstrated that T_{EX} signatures were highest in basal tumors and slightly higher in luminal B tumors than luminal A tumors (FIG. 14A). As tumor infiltrating T_{EX} have been shown to be specific for somatic mutation derived neoantigens, we next investigated if increased tumor mutation burden coincided with increased T_{EX} in BC patients (43). Surprisingly, within both TNBC and ER+ METABRIC cohorts, T_{EX}hi tumors had a statistically decreased number of somatic mutations detected (FIGS. 14B-14C). However, the difference in mean mutation burden between T_{EX}hi and T_{EX}lo tumors was only 2 somatic variations. To confirm the lack of association between T_{EX} infiltration and high tumor mutation burden, we next performed tumor mutation load (TML) analysis on our own ER+ tumor tissues using a targeted TML panel. Again, observed TML did not correlate in any way with the T_{EX} frequencies of CD8⁺ TILs identified by flow cytometry (FIG. 14D). Taken together these observations indicate that increased levels of T_{EX} CD8⁺ TILs in BC patients cannot necessarily be accounted for by increased tumor mutation burden, although we do not discount the possibility that T_{EX} CD8⁺ TILs may be neoantigen specific.

CD8A expression and T_{EX} signature expression showed a modest positive correlation in both TNBC (R=0.6) and ER+(R=0.5) METABRIC tumors, revealing that high levels of CD8⁺ T_{EX} could be found in tumors with both hi and low levels of CD8⁺ T cells (FIG. 5A-5D). To investigate potential divergent contributions of overall CD8⁺ T cell infiltration and CD8⁺ T_{EX} infiltration we then stratified ER+ and TNBC tumors into CD8hi or CD8lo and T_{EX}hi or T_{EX}lo based off top 25% and bottom 25% cutoffs (FIGS. 14E-14F and 14H-14I). As expected, TNBC patients with CD8hi tumors had marked increased in survival as compared to those with CD8lo tumors (FIG. 5E). However, in ER+BC, patients with CD8hi tumors and CD8lo tumors demonstrated no differences in survival (FIG. 5F). TNBC patients with T_{EX}hi (top 25%) tumors had no improved survival relative to those with T_{EX}lo (bottom 25%) tumors (FIG. 5G). In stark contrast, ER+ patients with T_{EX}hi tumors had significantly reduced survival (FIG. 5H).

We next set out to reconcile our observations regarding CD8⁺ T_{EX} and overall CD8⁺ T cell infiltration by assessing survival in the context of both variables. For survival analysis in the context of both CD8⁺ T cells and T_{EX}, we further stratified tumors into four groups: CD8hiT_{EX}hi, CD8hiT_{EX}lo, CD8loT_{EX}hi, CD8loT_{EX}lo (FIGS. 14G, 14J). TNBC patients with CD8hiT_{EX}hi and CD8hiT_{EX}lo tumors demonstrated the best survival (FIG. 5I). Strikingly, in ER+ patients with CD8hiT_{EX}lo and CD8loT_{EX}lo tumors demonstrated the best survival (FIG. 5J). We next performed multivariate analysis of these gene signatures to confirm our observed contrast in the contribution of CD8⁺ T cell infiltration and T_{EX} infiltration to survival in TNBC and ER+BC patients. In TNBC patients increased overall survival was primarily driven by increased CD8A expression and to a lesser degree T_{EX} expression (FIG. 5K). In ER+BC patients, again survival had no association with CD8A expression and significantly decreased as T_{EX} expression increased in ER+BC patients (FIG. 5L). For context we compared hazard ratios to gene expression of CD3G and PTPRC (CD45). We found our T_{EX} signature to be significantly more predictive of outcome than immune (PTPRC) or T cell infiltration levels alone (CD3G or CD8A) in ER+BC patients.

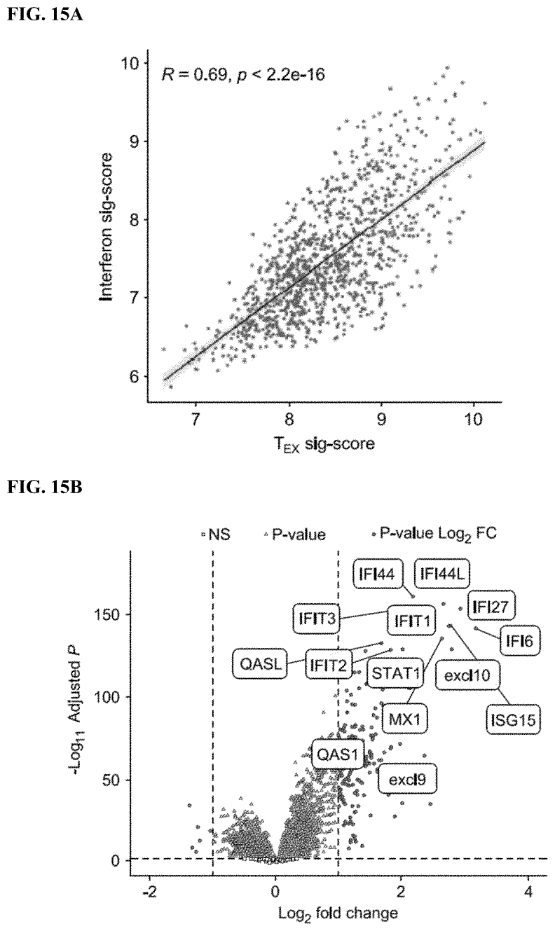

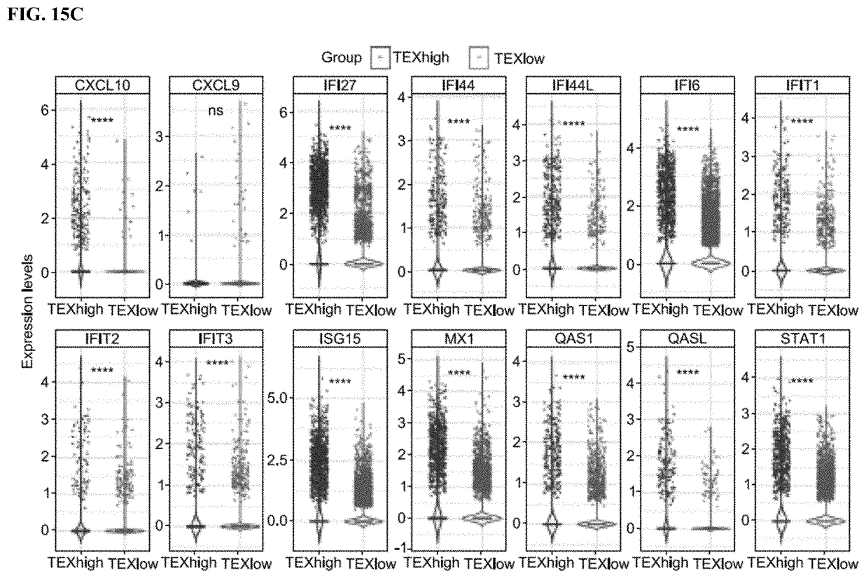

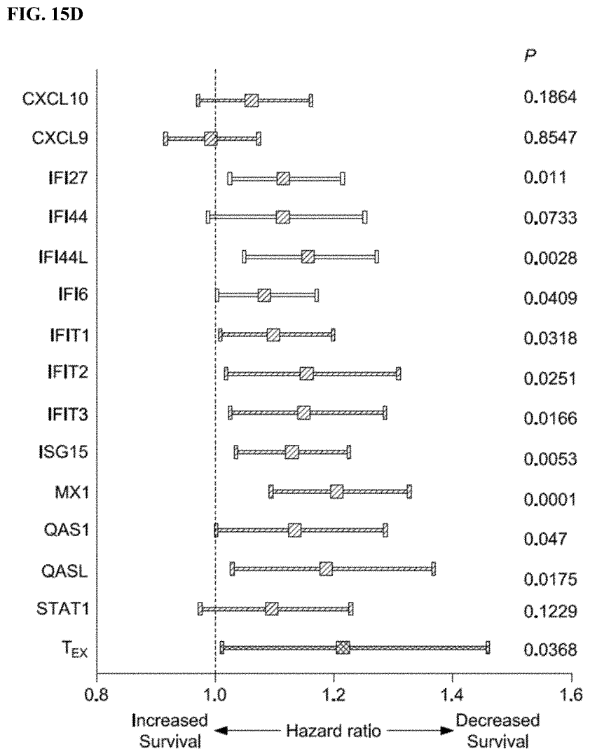

Increased expression level of interferon response genes has been associated with worse patient outcomes in ER+BC (44). We continued to explore the relationship between tumor interferon signaling, the presence of T_{EX}, and survival in ER+BC patients. In METABRIC ER+BC patients we found a strong correlation between the IFNγ tumor signature and our T_{EX} signature (FIG. 15A). In comparing T_{EX}hi and T_{EX}lo ER+BC tumors by differential gene expression, several of the most upregulated genes in T_{EX}hi tumors are involved in IFNγ signaling and response, including CXCL10, CXCL9, IFI27, IFI44, IFI44L, IFI6, IFIT1, IFIT2, IFIT3, ISG15, MX1, OAS1, OASL, and STAT1 (FIG. 15B). We then assessed these IFNγ-associated genes within cancer cells specifically by using internal single cell sequencing data from ER+BC tumors in which we knew the fraction of T_{EX} CD8⁺ TILs as determined by flow cytometry. Again, we categorized tumors as T_{EX}hi and T_{EX}lo by being above or below the overall median for % T_{EX} of CD8⁺ TILs (8%). All of the 14 IFNγ-associated genes we examined, with the exception of CXCL9, were found to be significantly elevated in cancer cells within T_{EX}hi tumors (FIG. 15C). Finally, a hazard ratio analysis of ER+BC patients found a significantly increased risk for lower survival with increased tumor expression of IFI27, IFI44, IFI44L, IFI6, IFIT1, IFIT2, IFIT3, ISG15, MX1, OAS1, and OASL (FIG. 15D). In summary, we identify a strong connection between T_{EX} CD8⁺ TILs, IFNγ signaling in breast cancer cells, and reduced overall survival in ER+BC patients.

Unfavorable Survival in Premenopausal Estrogen Receptor Positive Breast Cancer Patients with High T_{EX} Tumor Infiltration

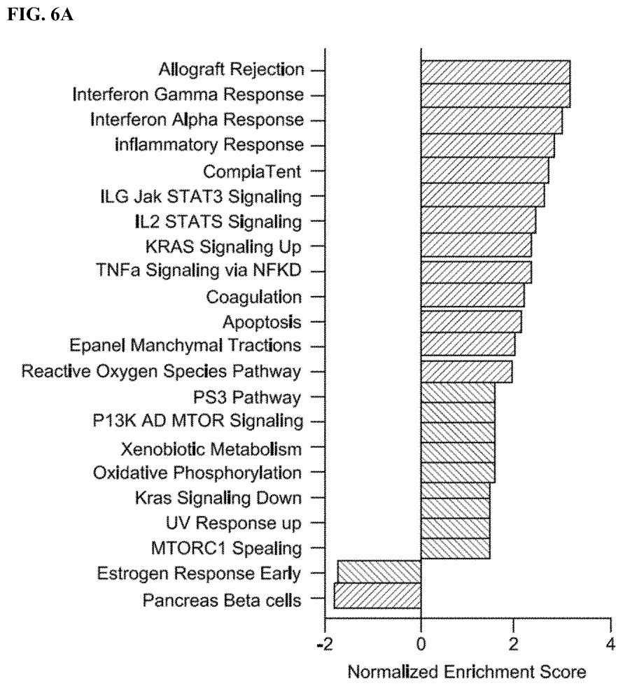

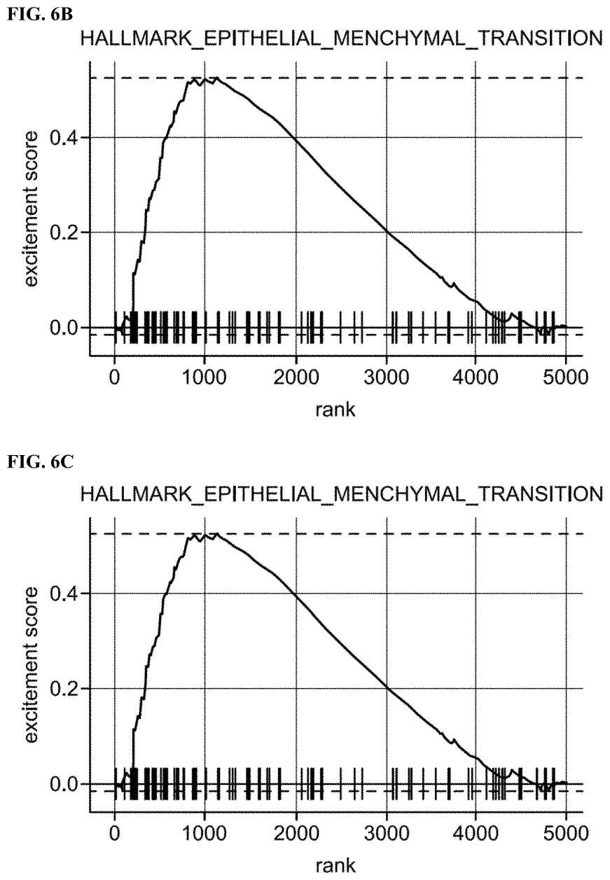

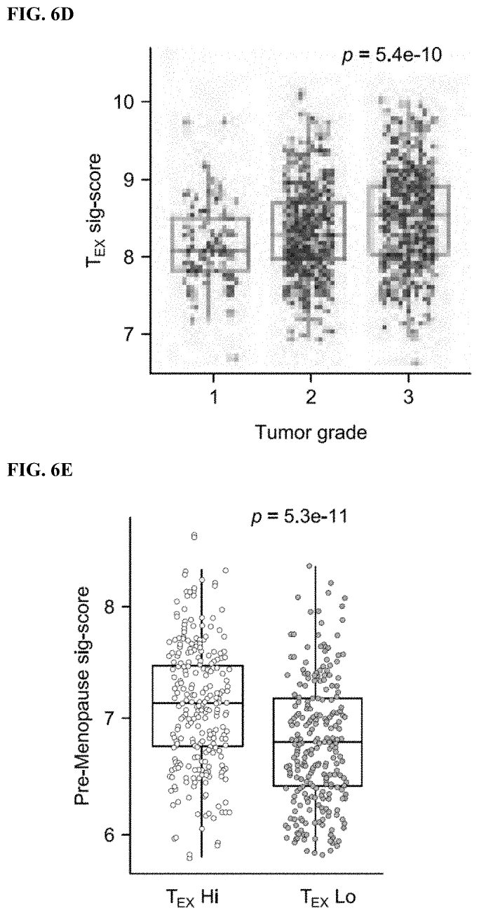

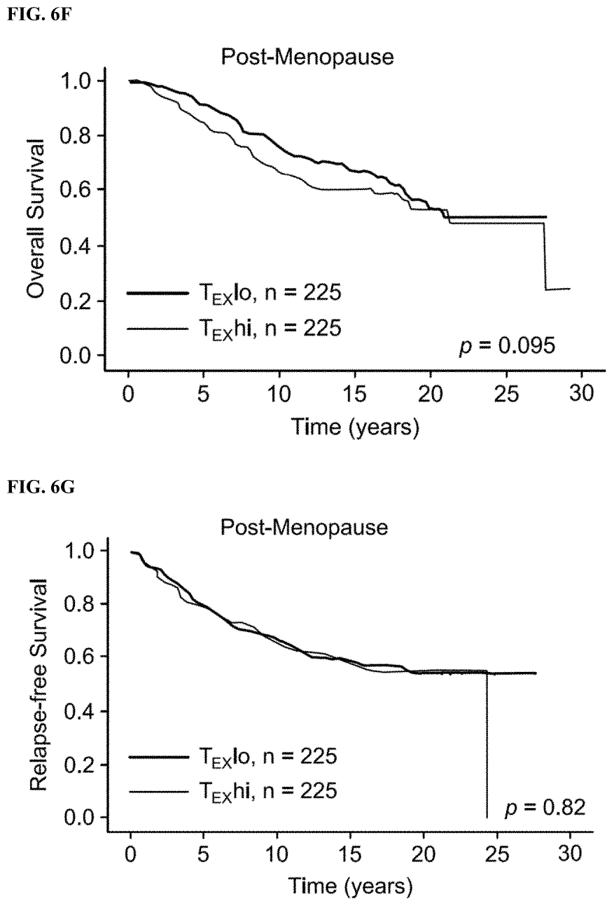

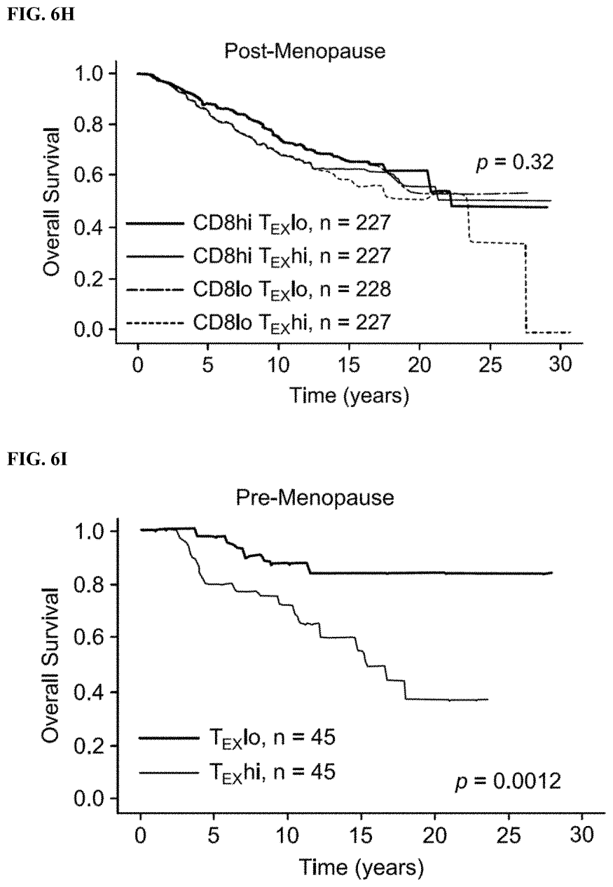

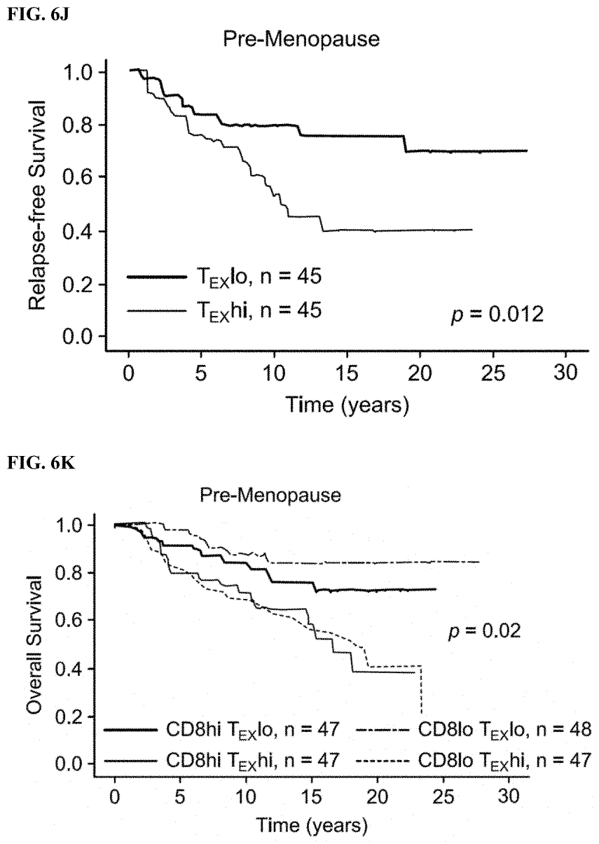

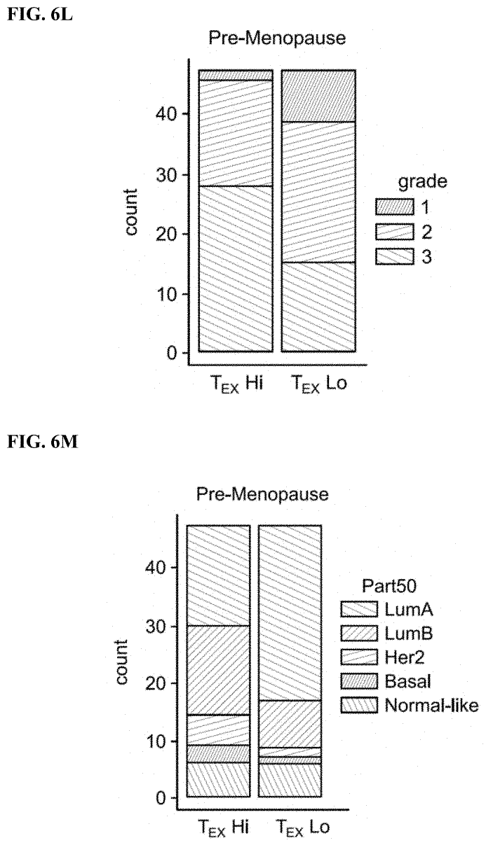

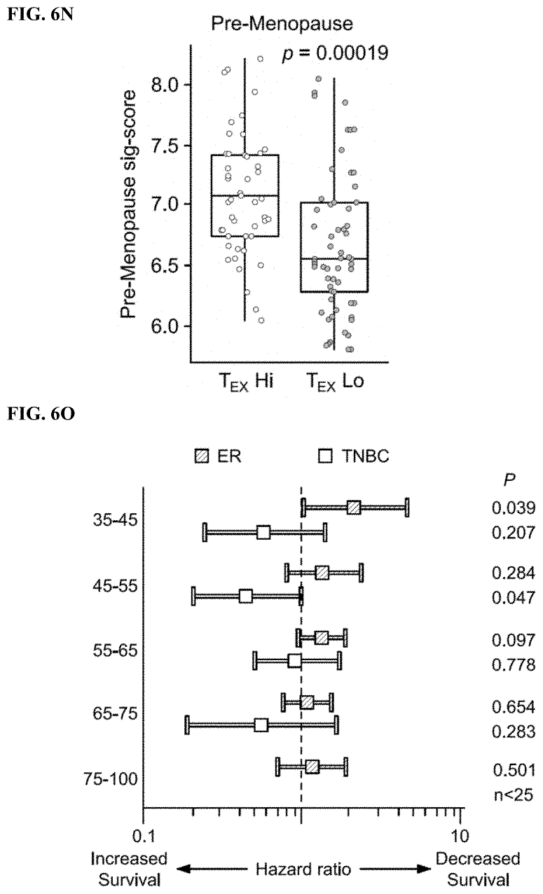

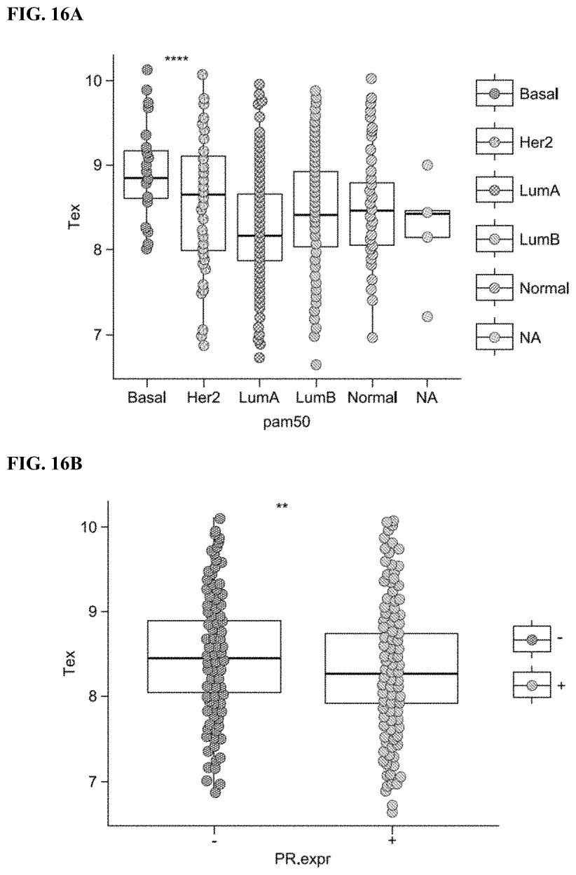

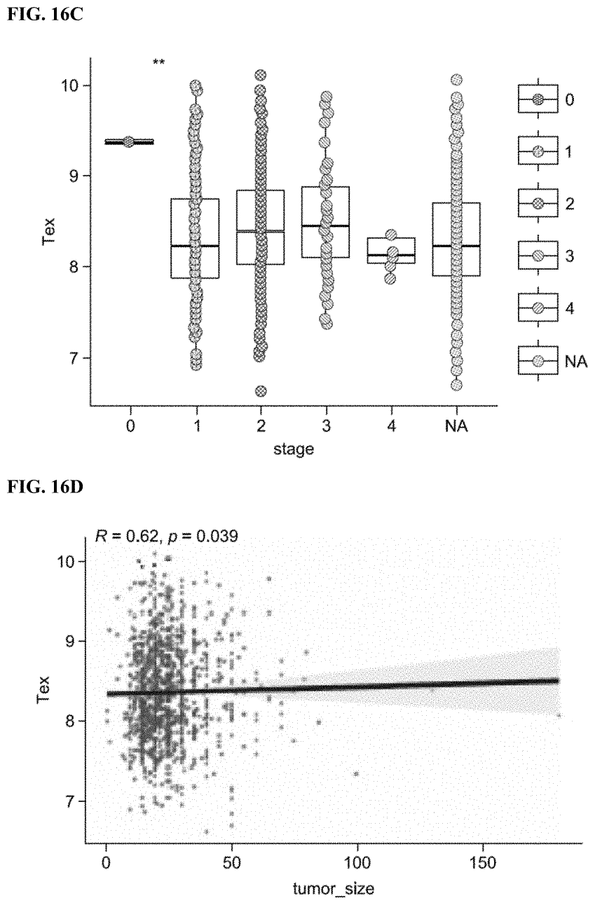

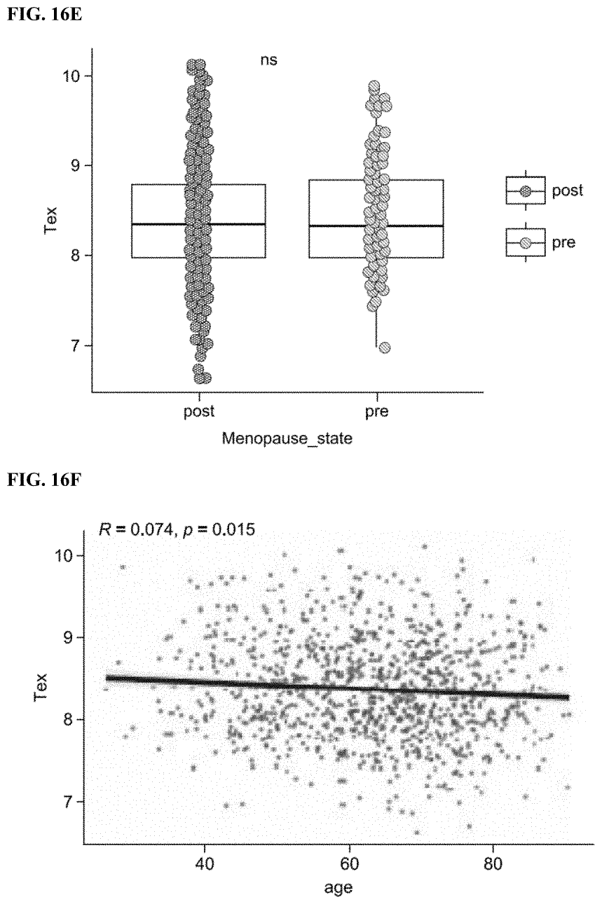

To further dissect features of T_{EX}hi ER+BC patient tumors, we examined The Cancer Genome Atlas (TCGA) repository data to validate and expand on our findings in the METABRIC cohort. Hallmark pathway analysis similarly found increased expression level of several immune-related pathways in ER+ tumors, including allograft rejection, interferon responses, and inflammatory responses (FIG. 6A). Intriguingly, our analysis of T_{EX}hi ER+ tumors also identified increased expression level of genes related to epithelial mesenchymal transition and decreased expression of early estrogen response genes, indicating the association between T_{EX} CD8⁺ TILs and more aggressive tumor features (FIGS. 6B-6C). Indeed, within the ER+ METABRIC cohort we found that T_{EX} signatures generally increased in ER+ tumors as the grade of the tumor increased, although high T_{EX} expression was still identified in both grade 1 and grade 2 tumors (FIG. 6D). Furthermore, T_{EX} signatures were increased in ER+ tumors with either Basal or Luminal B PAM50 subclassification and tended to have diminished progesterone receptor expression (FIGS. 16A-16B). Importantly, we also found that as compared to T_{EX}lo ER+ tumors, T_{EX}hi ER+ tumors had a significantly increased proliferation signature (FIG. 6E). No associations were identified between the presence of T_{EX} CD8⁺ TILs patient stage, tumor size, menopause state, or age (FIGS. 16C-16F).

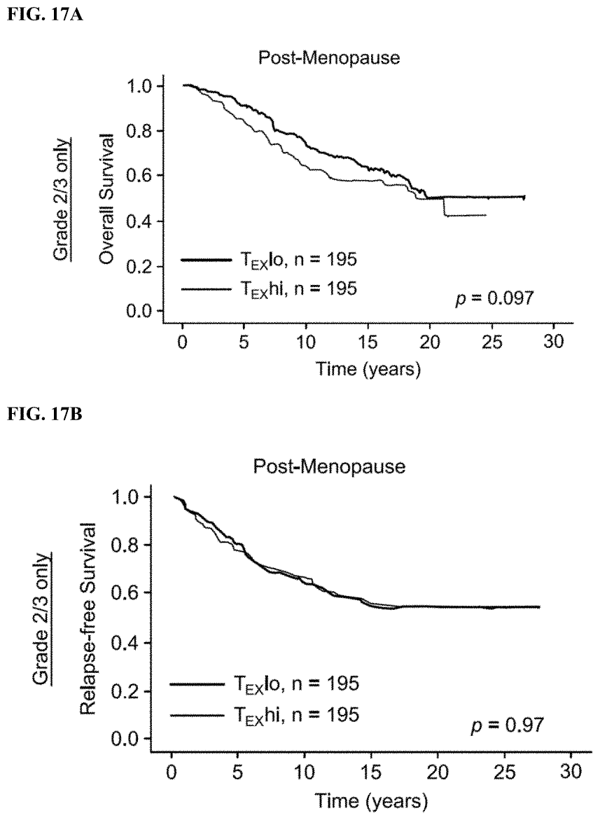

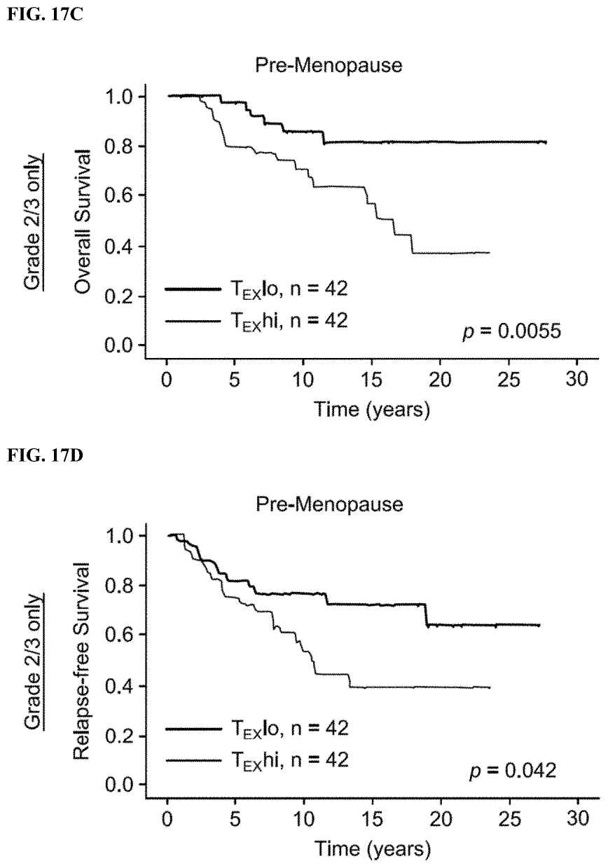

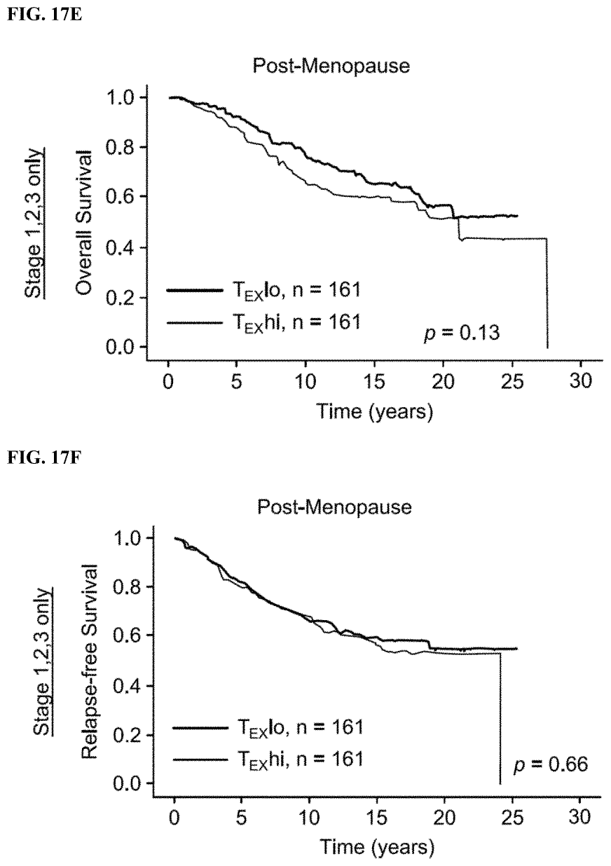

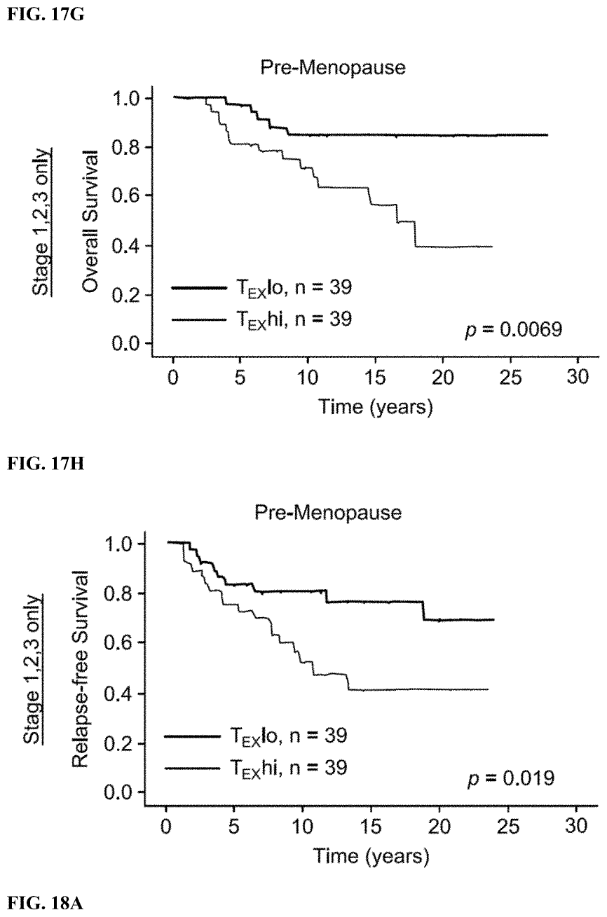

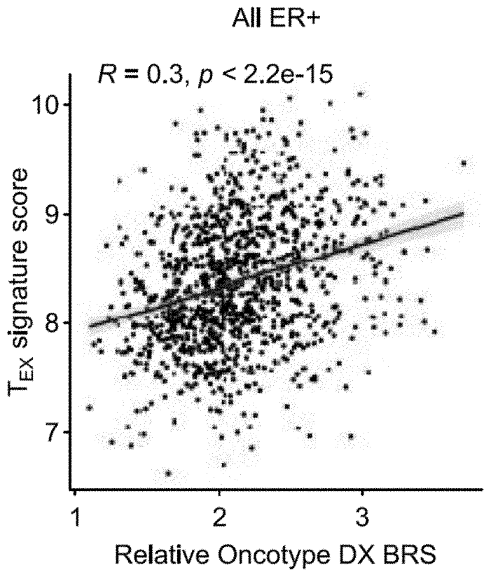

Given the findings of a more aggressive tumor phenotype in T_{EX}hi ER+ tumors, including decreased estrogen response related gene expression, we hypothesized that survival characteristics may be different in premenopausal and postmenopausal ER+BC patients. Using the menopausal status as defined by METABRIC (cutoff of 50 years old), we separately examined overall survival and relapse-free survival in premenopausal and postmenopausal ER+BC patients. In postmenopausal women, overall survival trended to be reduced in patients with T_{EX}hi tumors (FIG. 6F). However, no significant differences in relapse-free survival were found (FIG. 6G). Similarly, dividing postmenopausal tumors into CD8hiT_{EX}hi, CD8hiT_{EX}lo, CD8loT_{EX}hi, and CD8loT_{EX}lo did distinguish significant survival differences (FIG. 6H). On the other hand, premenopausal women with T_{EX}hi tumors had dramatically reduced overall survival and relapse-free survival as compared to premenopausal women with T_{EX}lo tumors (FIGS. 6I-6J). Further analysis of CD8hiT_{EX}hi, CD8hiT_{EX}lo, CD8loT_{EX}hi, and CD8loT_{EX}lo groups showed again that diminished survival was strictly associated with a T_{EX}hi phenotype, regardless of being CD8hi or CD8low (FIG. 6K). As clinical presentation of premenopausal and postmenopausal women may vary, we repeated and validated our survival findings in both Grade 1 and 2 only ER+ patients and Stage IV excluded ER+ patients (FIG. 17). Within premenopausal patients, T_{EX}hi tumors were increasingly composed of higher grade and Luminal B, HER2+, and basal molecular subset tumors, highlighting heterogeneity in the features of T_{EX}hi tumors (FIGS. 6L-6M). More striking was a highly increased proliferation signature in premenopausal T_{EX}hi tumors as compared to premenopausal T_{EX}lo tumors (FIG. 6N). A hazard ratio analysis of survival risk imparted by our T_{EX} signature identified women aged 35-45 as a group with the lowest overall survival and that T_{EX} associated survival risk steadily declined with age (FIG. 6O). Together these findings connect significantly reduced survival in younger, premenopausal women that can be defined by high infiltration of T_{EX} CD8⁺ TILs.

High Expression of a T_{EX} Signature Identifies High Risk Premenopausal Patients with Intermediate Oncotype Dx® Breast Recurrence Scores

Additional biomarkers to guide clinical care of early-stage ER+BC patients is a growing need. Gene expression testing to evaluate risk of recurrence is now standard of care for early-stage breast cancer patients. A prominent example of such gene expression testing is the Oncotype DX® Breast Recurrence Score® (BRS). Currently, treatment strategies for ER+BC patients with intermediate BRSs are less clear. With this in mind, we next hypothesized that added stratification of ER+ tumors by our T_{EX} signature could provide additional prognostication value in the context of intermediate Oncotype DX® BRS patients.

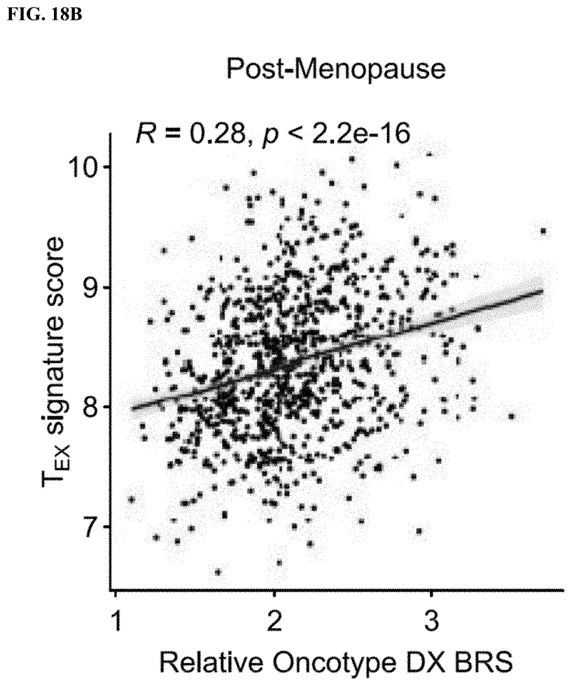

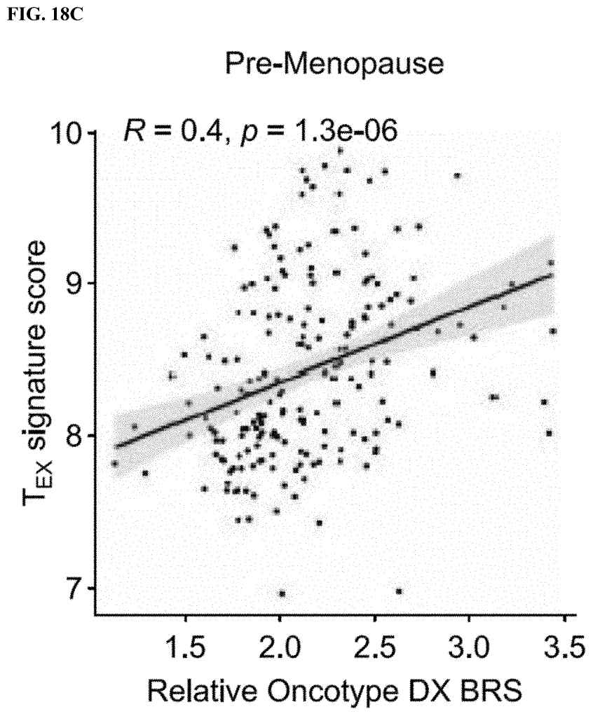

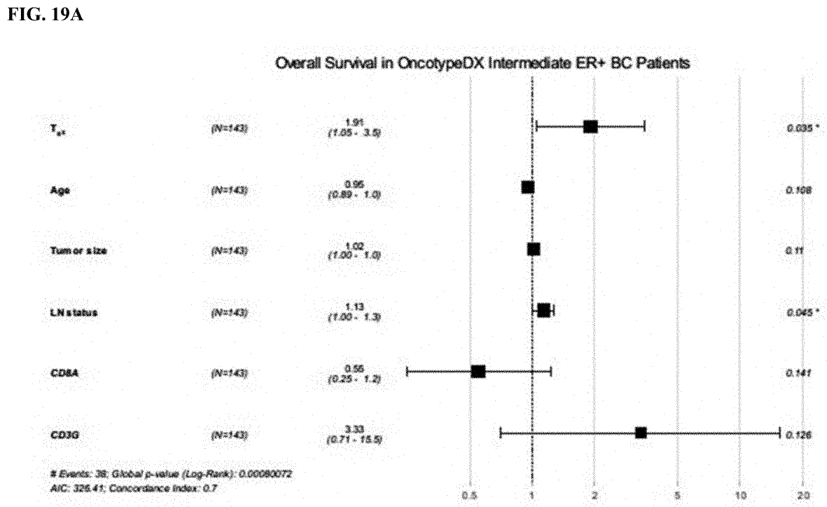

Without exact BRSs available, we utilized the gene expression of the Oncotype DX® 21 gene assay to calculate a BRS signature (45). T_{EX} signature expression and a relative BRS were weakly associated (R=0.3) in all, postmenopausal only, or premenopausal only METABRIC ER+ patients (FIG. 18). We did, however, observe that T_{EX}hi tumors appeared to be a subset of tumors with an intermediate BRS, suggesting that the T_{EX} signature could be used to further segregate these patients into distinct survival outcomes.

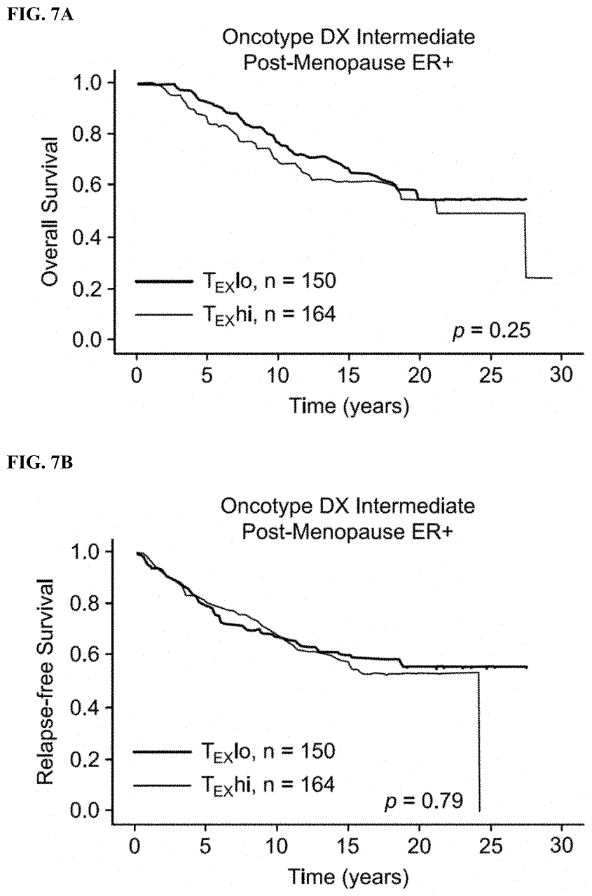

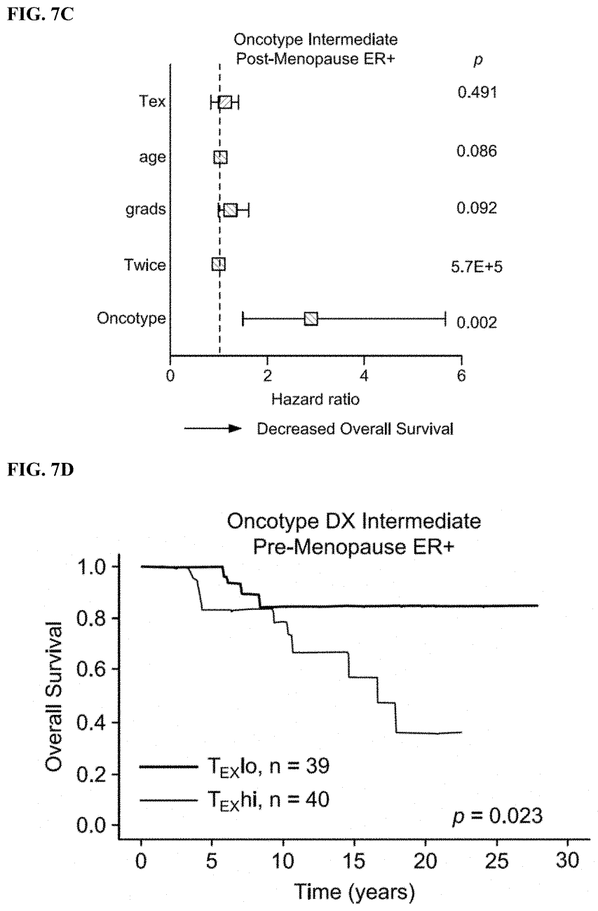

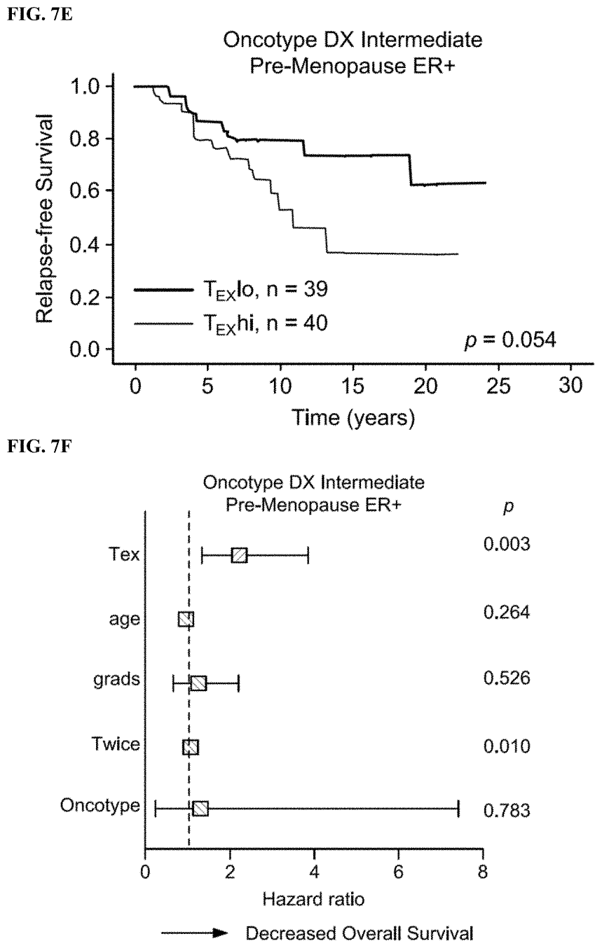

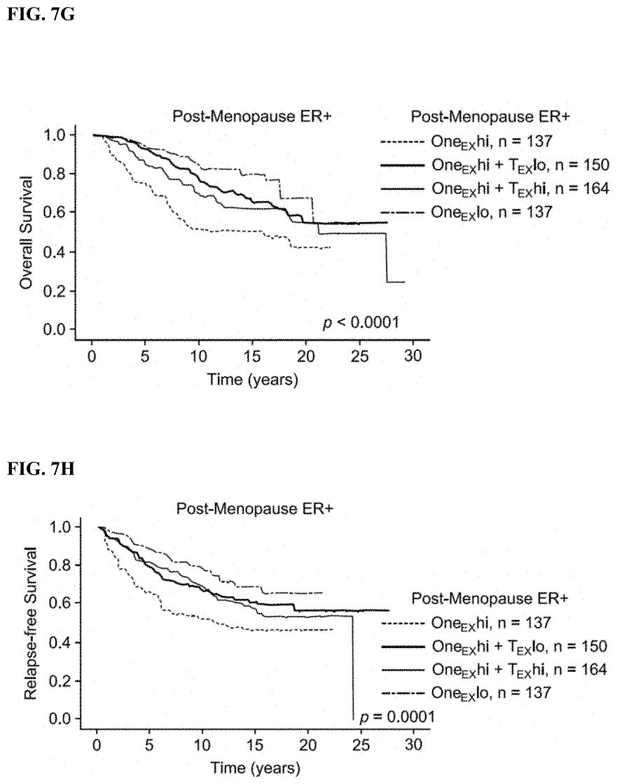

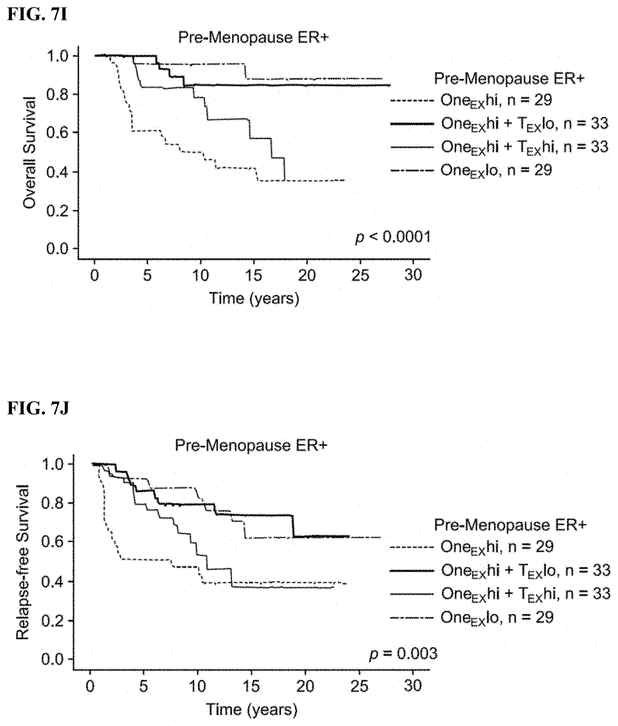

To investigate this, we next defined METABRIC ER+ patients as having a high (top 15%) Oncotype DX® BRS (Onc_{DX}hi), intermediate (middle 70%) Oncotype DX® BRS (Onc_{DX}int), or low (bottom 15%) Oncotype DX® BRS (Onc_{DX}lo) based off observable distributions amongst patients and published frequencies of these clinical phenotypes (46). Within postmenopausal Onc_{DX}int patients, T_{EX}hi and T_{EX}lo tumors did not display significant differences in overall survival or relapse-free survival (FIGS. 7A-7B). Additionally, multivariate analysis did not show any significant influence of T_{EX} signature expression on overall survival (FIG. 7C). In contrast, within premenopausal Onc_{DX}int patients, patients with T_{EX}hi tumors demonstrated significantly reduced overall survival and relapse-free survival as compared to those with T_{EX}lo tumors (FIGS. 7D-7E). Multivariate analysis further showed that increased T_{EX} signature expression significantly associated with decreased overall survival and more so than patient age, tumor grade, tumor size, or even Oncotype DX® BRS in premenopausal Onc_{DX}int patients (FIG. 7F).

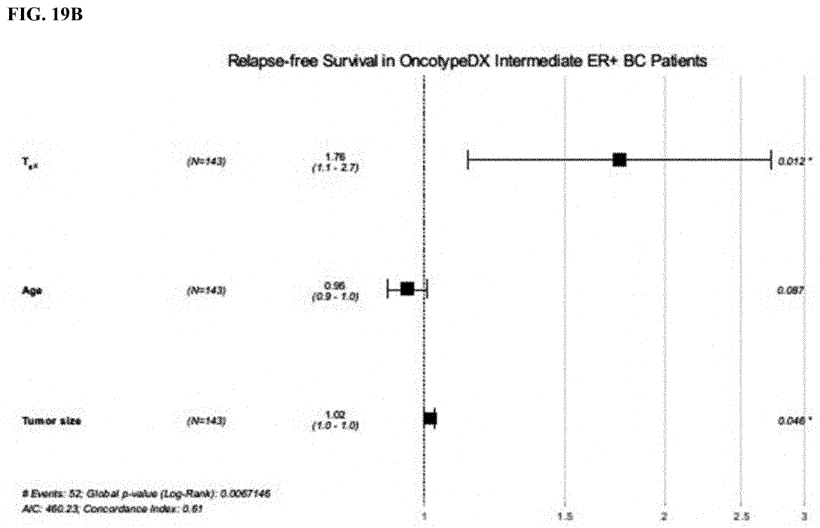

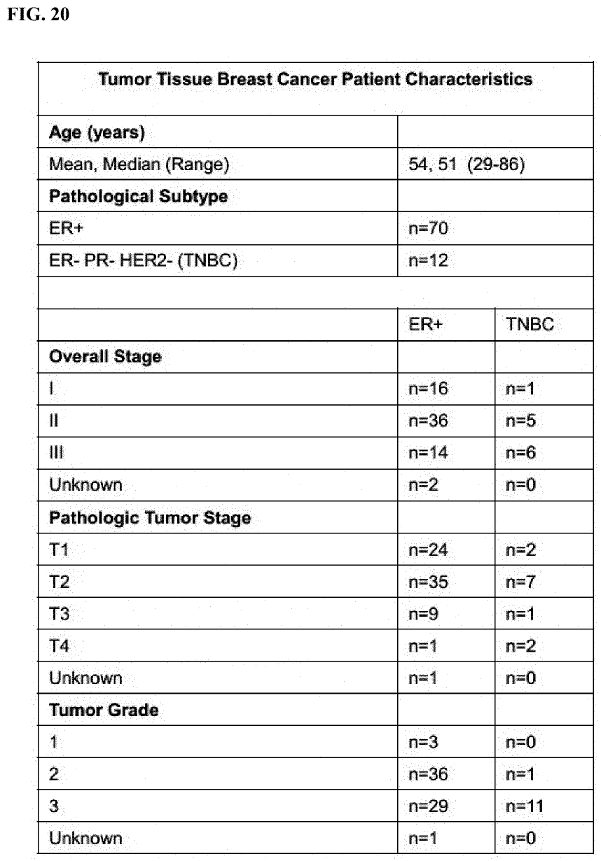

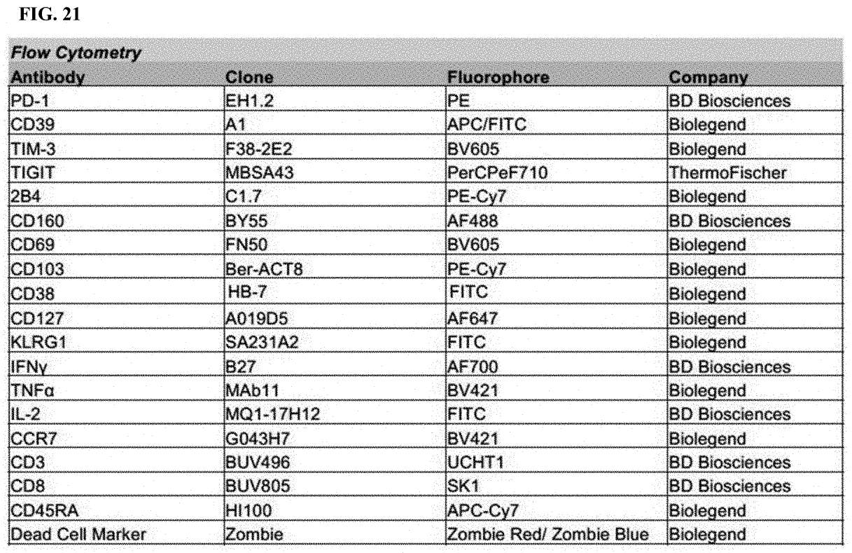

We next performed a stepwise model selection to prune the multivariate to estimate hazard ratios for survival (FIG. 19). To ensure that the influence of our T_{EX} signature was associated with survival independently from overall immune and T cell infiltration we also included gene expression of CD8A, CD3G, and PTPRC in addition to patient age, tumor grade, tumor size, and lymph node status. Of all variables, T_{EX} was found to be the most influential for both overall survival and relapse-free survival.

For overall survival, each additional unit of T_{EX} signature is associated with a 91% (p-value=0.03) increase in risk for Onc_{DX}int patients. For relapse-free survival, each additional unit of T_{EX} signature is associated with a 76% (p-value=0.01) for Onc_{DX}int patients.

Our findings demonstrated the value of using our T_{EX} signature to further prognosticate Onc_{DX}int patients. We therefore next examined patient survival characteristics in the context of four subgroups distilled from Oncotype DX® BRS and T_{EX} signature expression: Onc_{DX}hi, Onc_{DX}int+T_{EX}lo, Onc_{DX}int+T_{EX}hi, and Onc_{DX}lo. In postmenopausal patients, Onc_{DX}hi patients and Onc_{DX}lo patients had the shortest and longest survival outcomes respectively, but Onc_{DX}int+T_{EX}hi patients did not demonstrate significantly different survival characteristics from Onc_{DX}int+T_{EX}lo patients (FIGS. 7G-7H). In premenopausal patients, Onc_{DX}hi patients again had the shortest survival outcomes, but Onc_{DX}int+T_{EX}hi patients demonstrated dramatically decreased survival characteristics as compared to Onc_{DX}int+T_{EX}lo patients (FIGS. 7I-7J). Surprisingly, Onc_{DX}int+T_{EX}lo patients had remarkably similar survival characteristics to Onc_{DX}lo patients. These findings establish a T_{EX} signature as a useful means to further segregate high-risk and low-risk patients within premenopausal Onc_{DX}int patients.

### DISCUSSION

In this study, we showed that CD8⁺ T_{EX} occur within a subset of human breast tumors. These CD8⁺ T_{EX} were identified by distinct phenotypic properties, including PD-1 and CD39 co-expression, increased checkpoint molecule expression, reduced CD127 expression, and reduced cytokine production capacity. Single cell sequencing revealed that CD8⁺ T_{EX} in BC patients are transcriptionally unique. Furthermore, we established that increased presence of CD8⁺ T_{EX} occurs in immunologically distinct tumors with increased expression level of IFNγ related genes including PD-L1. We showed that despite signs of increased immune activation, ER+BC patients with a high CD8⁺ T_{EX} signature experience decreased survival. Survival was found most dramatically reduced in premenopausal ER+BC patients, identifying an important connection between anti-tumor immunity and menopausal status. Lastly, we demonstrate the clinical utility in using our T_{EX} signature to identify premenopausal Onc_{DX}int patients with decreased survival outcomes.

BC tumors, which predominantly have a low mutation burden, are widely viewed as non-immunogenic (47). We found that CD8⁺ T_{EX} can be identified in a subset of both TNBC and ER+ breast tumors, indicating tumor antigen recognition even in ER+ tumors. Phenotyping of these T_{EX} corroborate robust activation and clonal expansion in response to antigen. Tumor immunogenicity is generally thought to be correlated with increased tumor mutation burden and the resulting neoantigen-driven T cell reactivity to cancer cells (48, 49). Indeed, tumor neoantigen specific and exhausted CD8⁺ T cells have been described (50). Surprisingly, we did not find a correlation between the presence of CD8⁺ T_{EX} TILs and tumor mutation burden. These findings do not preclude the possibility that CD8⁺ T_{EX} in BC patients are neoantigen specific and highlight the need for further work to dissect antigen specificity in BC.

Our data connect the presence of CD8⁺ T_{EX} with an IFNγ rich TME and reduced survival in ER+BC patients. Other groups have also found evidence for immune activation in ER+ breast tumors. Wagner et al. showed that increased frequencies of PD-1⁺ CD38⁺ CD8⁺ T cells, which we demonstrate as CD8⁺ T_{EX}, correlate with the presence of PD-L1⁺ tumor associated macrophages in high-grade ER+ tumors (51). Mirroring our CD8⁺ T_{EX} signature analysis, Thorsson et al. demonstrated IFNγ signature enriched tumors to be most frequent in TNBC, followed by luminal B BC and then luminal A BC (52). We find that increased T_{EX} associates with reduced overall survival in ER+BC patients, but not TNBC patients. This further depicts ER+BC and TNBC as having substantially different features of immune-cancer interaction. Standard of care therapy, average time to relapse, and cancer cell biology are clearly different between these BC subsets and may play a role in these differential outcomes (53). Further studies exploring the development of CD8⁺ T_{EX} in the context of breast cancer neoadjuvant chemotherapy and adjuvant therapy regimens may reveal mechanisms for the survival characteristic differences between ER+BC and TNBC patients.

Increased IFNγ in the TME is generally considered to reflect an active anti-tumor immune response beneficial to patient outcomes. However, we demonstrate that high levels of CD8⁺ T_{EX} and IFNγ denote poor outcomes in ER+BC patients, most significantly in premenopausal patients in which circulating estrogen levels are highest (54). Our evidence therefore lends support to other findings of the potential pro-tumorigenic role of IFNγ signaling (55). In support of this, increased expression level of interferon response genes and JAK-STAT signaling genes have been found in both chemotherapy and tamoxifen resistant ER+ tumors (44, 56, 57). Additionally, phosphorylated STAT1 has been shown to be increased in premenopausal ER+BC patients with worse survival (58). Although the exact mechanism for the relationship between increased tumorigenesis and interferon signaling in ER+ tumors is not yet clear, there is evidence that STAT1 and estrogen receptor signaling may synergize for enhanced cancer cell proliferation (59). Our results indicate that T_{EX} CD8⁺ TILs inadvertently yield a rich source of IFNγ in the TME that is pro-tumorigenic and pro-metastatic in the context of ER+BC tumors. We demonstrate evidence for this by finding strong correlations between T_{EX}, increased tumor proliferation, increased tumor grade, and decreased survival in ER+BC patients. A novel therapeutic strategy that targets interferon signaling may be a viable approach for T_{EX}hi ER+ tumor BC patients (60).

Standard of care for early-stage ER+BC patients is currently guided by hormone receptor expression, pathological tumor features, and genomic testing such as the Oncotype DX® Breast Recurrence Score® (61). Patients with high Oncotype Dx® breast recurrence scores have significantly increased risk of relapse and benefit from chemotherapy intervention (45). However, treatment strategies for approximately 70% of ER+BC patients with intermediate Oncotype Dx® breast recurrence scores are less clear and clinical outcomes differ further between premenopausal and postmenopausal women (62). Here we demonstrate the use of a T_{EX} gene signature to further select for high-risk patients amongst those categorized as premenopausal and Onc_{DX}int. Our results indicate that early stage premenopausal Onc_{DX}int T_{EX}hi patients are a high-risk cohort that may benefit from adjuvant or neoadjuvant therapies. Future preclinical and clinical studies are needed to dissect the relationship between CD8⁺ T_{EX}, metastasis development, and response to therapy in the context of both hormone receptor status and menopausal status.

Methods

Human Samples

Tissues were obtained from consented BC patients undergoing standard of care therapy at City of Hope. Patient characteristics are summarized in Supplementary Table 1. Classification of tumor samples as ER+, PR+, or HER2+ was performed by clinical pathologists. NCBTs were composed of tissue from high-risk patient prophylactic mastectomies, contralateral breast from BC patient mastectomies, or tumor adjacent tissue. Due to limited cell numbers obtained from patient tumor samples, not all analyses shown were performed on the same samples. Tissue samples were provided by the City of Hope Biospecimen Repository, which is funded in part by the National Cancer Institute. Other investigators may have received specimens from the same patients.

Sample Processing

Patient peripheral blood was obtained by venipuncture using heparin collection tubes, transported at room temperature from the clinic to the lab, and processed within 6 hours of drawing. PBMCs were isolated via Ficoll-Paque Separation (GE Healthcare) following the manufacturer's instructions. Solid tissue specimens were collected by surgical resection and collected in tubes containing cold HBSS (Life Technologies, Thermo Fisher Scientific) and transported on ice to the laboratory for processing within one hour of surgery. Tumor negative lymph nodes were mechanically dissociated and filtered into single cell suspensions. Tumor, tumor positive lymph nodes, and NCBT tissues were minced into pieces, mechanically dissociated with a gentleMACS Dissociator (Miltenyi Biotec), and enzymatically treated with 0.2 Wunsch units/ml Liberase™ (Roche) and 10 units/ml DNase (Sigma) in RPMI for up to 1 h as needed. If necessary, red blood cell (RBC) lysis was performed using RBC Lysis Buffer (Biolegend).

Flow Cytometry

Single cell suspensions were stained at RT in 2% FBS in PBS. For cytokine production assays, cells were stimulated with 50 ng/ml PMA (Sigma) and 1 μg/ml ionomycin (Sigma) in the presence of Golgi Plug (Biolegend) for 4 hours. Overnight fixation was performed as needed with IC Fixation Buffer (eBioscience). Fixation and permeabilization was performed with BD Cytofix/Cytoperm buffers for intracellular cytokine staining. Antibody cocktails were diluted in Brilliant Violet Buffer (BD Biosciences) when using two or more Brilliant Violet labeled antibodies. Samples were acquired using a BD Fortessa operating FACS Diva 6.1.3. Photomultiplier tube voltages were set using BD CS&T beads. Compensation was calculated using single stained OneComp compensation beads (eBioscience). Samples were stained with fluorescently tagged antibodies detailed in Supplementary Table 2. Antibodies were titrated for optimal signal to noise ratio prior to use. Flow cytometry analysis was performed using Flowjo v10.6. All samples were gated on single cells, lymphocytes, and CD3⁺ CD8⁺ populations (Supplemental FIG. 1). Histograms and zebra plots are used to display data.

T Cell Single Cell Sequencing and Analysis

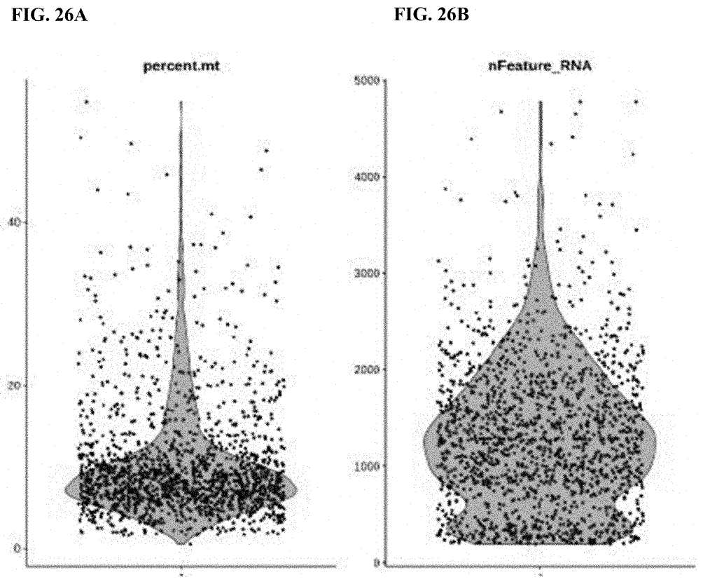

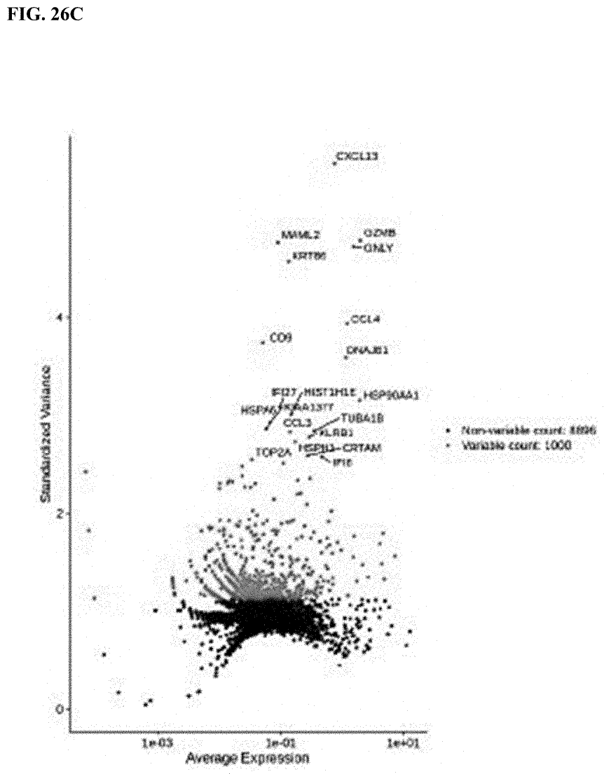

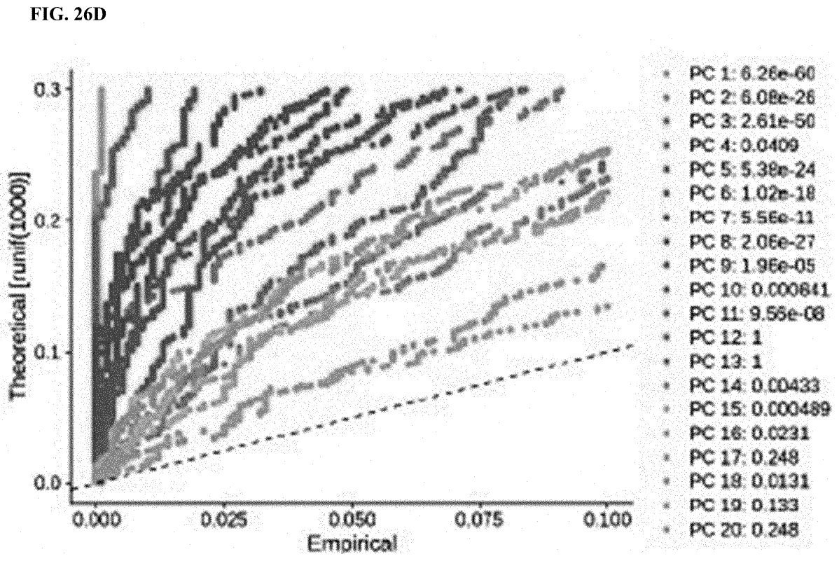

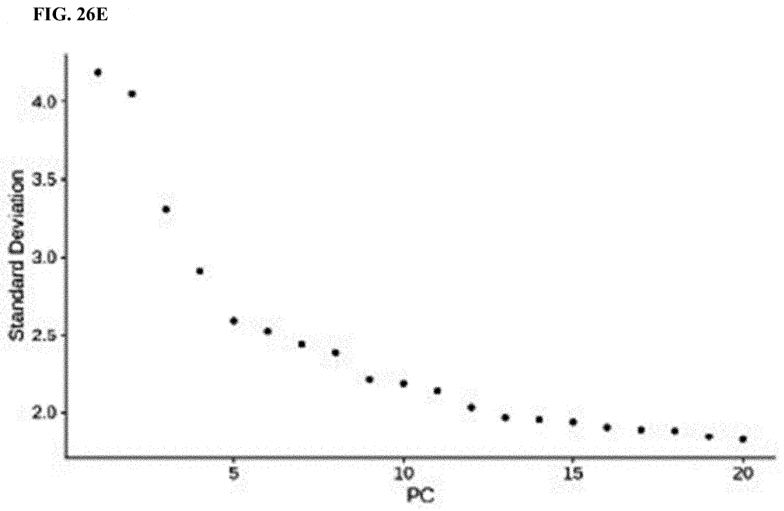

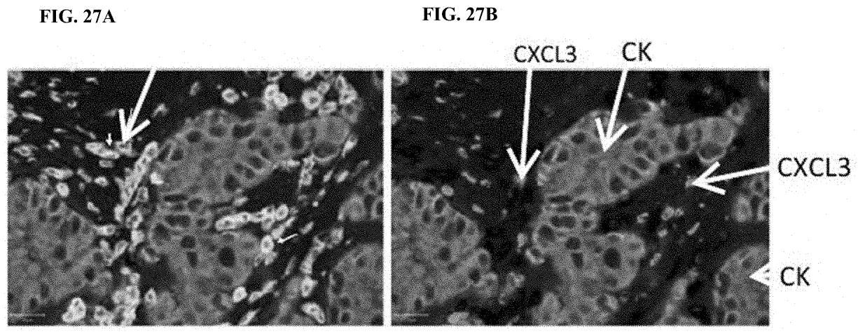

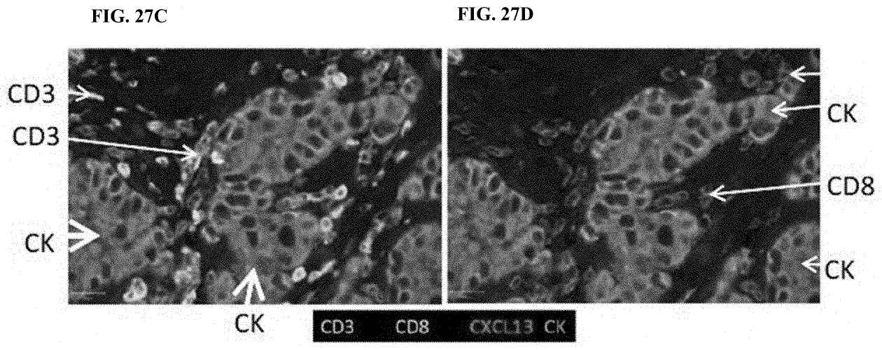

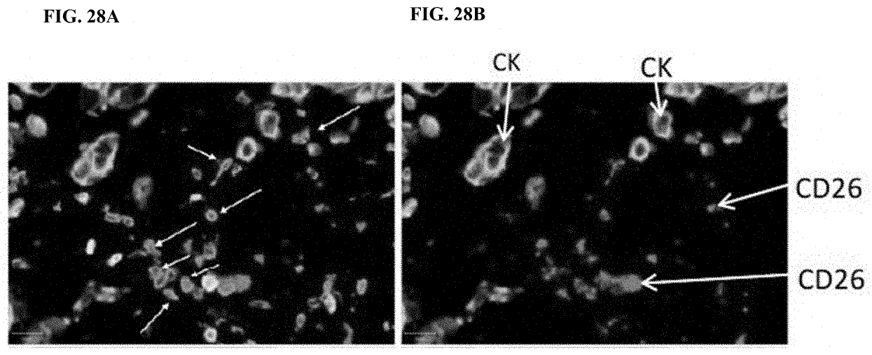

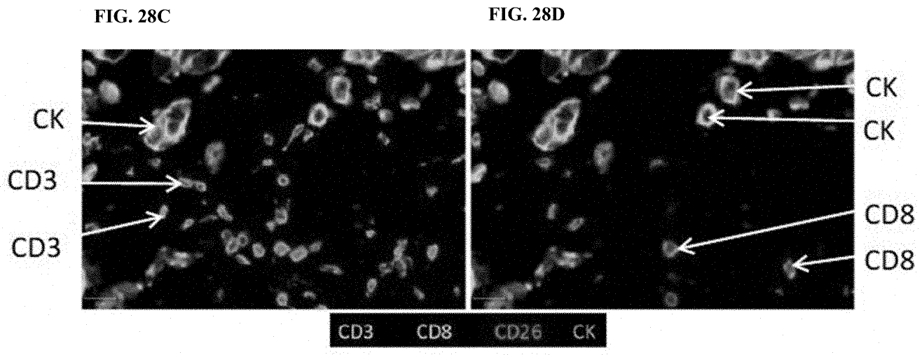

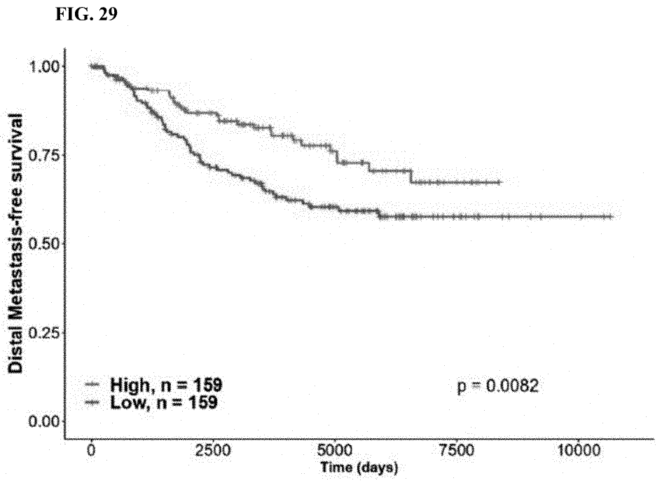

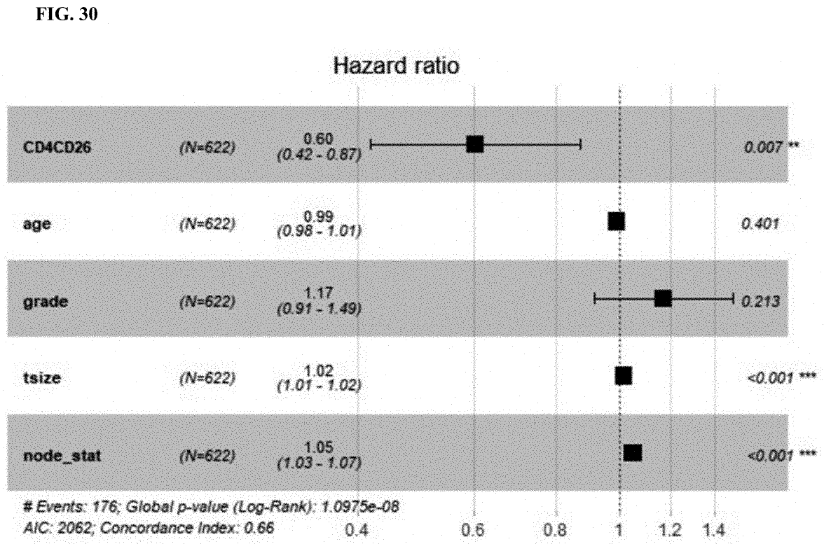

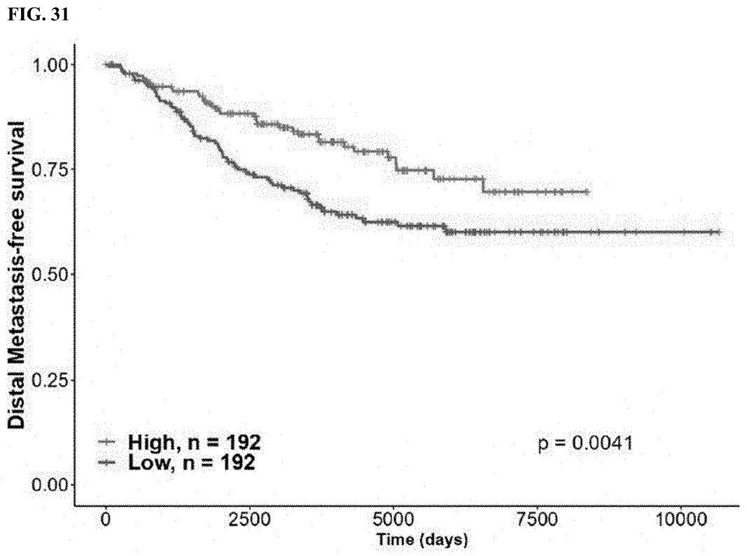

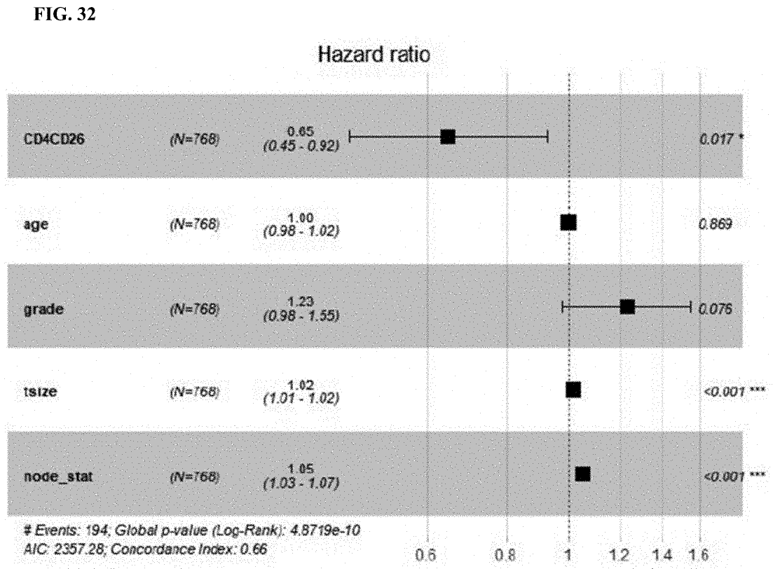

CD8⁺ T cells were stained with monoclonal antibodies for CD8, PD-1, CD39, CD103, CD69, CD137, and CCR7 as described above and then single-cell index sorted using a BD ARIA III FACS system into BD Genomics Precise WTA 96 well plates for whole transcriptome analysis. Single-cell libraries were prepared as recommended. Sequencing was performed on an Illumina HiSeq 2500 with an estimated 250,000 reads per cell. The raw counts were imputed by scImpute R package (v0.0.7) with kcluster=5(64). The imputed counts were analyzed by Seurat R package (v3.1.4)(65). Briefly, nonviable cells (defined as the cells in which more than 20% expressing genes are mitochondrial genes) were removed (FIG. 26A). In addition, the potential empty well or duplets (defined as cells in which less than 200 genes are expressed or more than 2500 genes are expressed, respectively) were also discarded for further analysis (FIG. 26B). The normalization was implemented by Seurat with default settings. The top 1,000 most variable protein-encoding genes were selected for principle component analysis (FIG. 26C). Based on the two heuristic methods in Seurat (modified Jack Straw procedure and ranking variance method, FIGS. 26D-26E), the top 12 principal components were used for the further non-linearly dimensional reduction (i.e., tSNE) and clustering analysis. Four major T cell clusters were found and visualized by tSNE projection. Based on the FACS markers and genetic markers, all the T cell clusters were annotated. The signature of T_{EX} was filtered from the genetic markers based on the log 2 fold changes (>1.0, i.e., larger than 2-fold change compared to the other T cells) and adjusted p-value (<0.10). Single cell sequencing data of T cells are deposited under the GEO accession number GSE190202.

Tumor Single Cell Sequencing and Analysis

Tumor single-cell RNA sequencing was implemented through 10×chromium platform with recommended procedures. CellRanger was used to align sequence reads to human genome and count the aligned transcripts for each cell. The raw counts for each tumor sample were directly filtered, normalized, and scaled by Seurat R package (v3.1.4) with the same parameters described above. The top 2,000 most variable genes were selected for sample integration and principle component analysis (PCA). All the tumor samples were integrated with standard integration workflow in Seurat (i.e., “FindIntegrationAnchors” and “IntegrateData” functions in Seurat with 50 dimensionality). PCA was implemented for the integrated data object. Based on the two heuristic methods in Seurat (modified Jack Straw procedure and ranking variance method), the top 20 principal components were used for the further non-linearly dimensional reduction (i.e., UMAP) and clustering analysis. The clusters with PTPRC⁻EPCAM⁺ cells were annotated as tumor cell cluster. The tumor cells were stratified into T_{EX}hi and T_{EX}lo groups based on the T_{EX} abundance with FACS. The normalized gene expression of genes of interests in tumor cells were compared between T_{EX}hi and T_{EX}lo groups with Wilcoxon rank sum test. Single cell sequencing data of T cells are deposited under the GEO accession number GSE190202.

Public Genomic Data Analysis

To evaluate the prognosis effect of T_{EX} in breast cancer, the Molecular Taxonomy of Breast Cancer International Consortium (METABRIC), one of the largest breast cancer multi-omics database, was downloaded with latest clinical information and normalized expression data (Illumina HT 12 platform) from European Genome-Phenome Archive (dataset ID: EGAD00010000210 and EGAD00010000211) (66, 67). A total 1,992 patients' data were obtained and organized. Based on the hormone receptor statuses, the ER+(all ER+ patients) and TNBC (ER− PR− HER2−) populations were stratified as 1098 and 269 patients respectively. The signature score of T_{EX} and other signatures was calculated using the sig.score function in genefu R package (v2.18.1) with default settings (68). The log 2 fold changes of genetic markers were considered as the weights in the signature. Based on the T_{EX} score and CD8A expression, the ER+ and TNBC cohorts were stratified as described in figure legends and text. The survival comparison and Kaplan Meier curves between groups were implemented by survminer (v0.4.8) and survival (v3.2-7) R packages with log-rank statistics. Hazard ratios were generated with a Cox Proportional-Hazards model in univariate or multivariate plots. The multivariate Cox regression analysis accounted for influence of age, tumor grade, tumor size, and nodal status or as described. The association between T_{EX} signature scores and molecular and pathological features were investigated by R (v3.6.2). A tumor proliferation score was calculated based on the expression of a 19 gene proliferation signature (69).

The gene expression data (including counts and FPKM-UQ) of breast cancer primary tumor samples of The Cancer Genome Atlas (TCGA) was downloaded from NCI-GDC data portal with the corresponding clinical information (including hormone receptor statuses and overall survival). Based on the hormone receptor statuses, 538 ER+ patients with primary tumors were found. The same approach described above was used to calculate the T_{EX} signature scores for TCGA-BRCA ER+ primary tumors with normalized expression data (FPKM-UQ). The ER+ patients were further stratified into T_{EX}hi and T_{EX}lo cohort with top 25% highest T_{EX} signature scores and bottom 25% lowest T_{EX} signature scores, respectively. The differential expression analysis for T_{EX}hi and T_{EX}lo groups was implemented by DESeq2 R package (v1.30.0) with the count data and recommended normalization procedure. The significantly differentially expressed genes were defined as the genes with adjusted p-value <0.05 and log 2 fold change <−1 or >1. These genes were further used to implement gene set enrichment analysis with the HALLMARK pathway sets (msigdbr package, v7.2) with fgsea R package (v1.16.0)(70). The “fgsea” function was used with log 2 fold changes as the rank score (stats), 1,000 permutations, and other recommended settings.

The CIBERSORTx was utilized to deconvolute the METABRIC expression data to 22 major immune cell types (LM22 signature) with standard data pre-processing procedure and 500 permutations for significance analysis (71, 72). The relative abundances of all the 22 major immune cell types between T_{EX} high and T_{EX} low cohorts were compared by Wilcoxon singed-rank test. The differential expression analysis between T_{EX} high and T_{EX} low cohorts of METABRIC expression data was implemented by limma R packages (v3.42.2) and visualized by EnhancedVolcano R package (v1.4.0) (73). The Gene Set Enrichment Analysis was implemented by GSEA software (v4.0.3) with latest Hallmark Molecular Signatures Database from Broad Institute (74, 75).

The in-house Oncotype Dx® scores for all the METABRIC ER+ breast tumors (n=1098) were calculated using the “sig.score” function in genefu R package (v2.18.1) with default settings. The weights of genes in Oncotype Dx® signature were calculated based on the Recurrence-Score algorithm described in Paik et al (45). The standard Oncotype Dx scores (0-100, Oncotype Dx® Breast Recurrence Score) were calculated using the “oncotypedx” function in genefu R package (v2.18.1) with default settings (62). A strong correlation between in-house and standard Oncotype Dx® scores was observed (r=0.89, p<0.01) and the stratification criteria for standard Oncotype Dx® high and low groups were aligned with 85% (scaled score >25) and 15% (scaled score <15) percentiles of the whole population of ER+ breast cancer patients (46). As in-house Oncotype Dx® scores were strongly correlated with the standard Oncotype Dx® scores and had a more similar scale to T_{EX} scores, the in-house Oncotype Dx® scores were used for the following analysis. Based on the T_{EX} score and in-house Oncotype Dx® scores, the ER+ cohort was stratified as described in figure legends and text. The survival comparison and analysis were implemented with the same statistics and packages for CD8A expression (details described above).

Nanostring Gene Expression Analysis.

RNA was extracted from 10 μm thick slices of unbaked formalin fixed paraffin embedded (FFPE) tissue using Qiagen miRNeasy FFPE kits. RNA transcripts were detected using Nanostring PanCancer Immune Panel with nCounter technology (Nanostring Technologies). RNA concentration was assessed with the Nanodrop spectrophotometer ND-1000 and Qubit 3.0 Fluorometer (Thermo Fischer Scientific). RNA fragmentation and quality control were further determined by 2100 Bioanalyzer (Agilent). Total RNA was hybridized overnight at 65° C. for 14 to 18 hours as per manufacturers' recommendations. Post-hybridization, probe-target mixture was purified by nCounter Prep Station and then quantified with nCounter Digital Analyzer. Quality control and normalization of data were performed with nSolver Analysis Software version 4.0 and the measured gene expression values were normalized to the geometric mean of 40 housekeeping genes. Advanced analysis was conducted using nCounter Advanced Analysis Software version 2.0.115. Heatmaps were generated using the ComplexHeatmap R package (v2.1.1). Nanostring genomic data are deposited under the GEO accession number GSE190169.

Tumor Mutation Load Assessment

DNA was extracted from 10 μm thick slices of unbaked FFPE tissue using Qiagen QIAamp DNA FFPE Tissue kits and measured using Qubit 3.0 Fluorometer (Thermo Fischer Scientific). Using 20 ng of DNA, the library was prepared following manufacturers' instructions. Once the libraries were generated, concentration was measured by qPCR using the Ion Library TaqMan® Quantitation Kit. Following qPCR, the libraries were calculated and pooled together at equal 50 pM concentration for templating on the Ion Chef using the Ion 540™ Kit-Chef (2 sequencing runs per initialization). The samples were then sequenced on the Ion GeneStudio™ S5 System. Ion Reporter™ Software was used for Mutation Load and Variant Profiling analysis.

Immunohistochemistry

FFPE tissue samples were sectioned at a thickness of 5 m, baked, and placed on positively charged glass slides. Slides were loaded on a Ventana Discovery Ultra (Ventana Medical Systems, Roche Diagnostics) automated IHC staining machine for deparaffinization, rehydration, endogenous peroxidase activity inhibition and antigen retrieval (pH 8.5). Antigens were sequentially detected and heat inactivation was used to prevent antibody cross-reactivity between the same species. Following each primary antibody incubation (CD8, clone SP57; PD-L1, clone SP263; CD20, clone L26), DISCOVERY anti-Rabbit HQ or DISCOVERY anti-Mouse HQ and DISCOVERY anti-HQ-HRP were incubated. The stains were then visualized with DISCOVERY ChromoMap DAB Kit, DISCOVERY Teal Kit and DISCOVERY Purple Kit, respectively, counterstained with hematoxylin (Ventana), and sealed with coverslips. Slides were imaged using the Vectra 3 automated quantitative pathology imaging system (Akoya Biosciences). Slides were then scored for percent tumor stroma infiltration by a board-certified pathologist. In order to normalize % T_{EX} infiltration abundance for Nanostring analysis in FIG. 4, % T_{EX} of tumor tissues were calculated as follows: (FACS % T_{EX})×(IHC % CD8).

Additional Statistics

Graphs and statistics were performed using Graphpad Prism 8 and specific R packages as described. Statistics described were generated using one-way unpaired student T tests, Wilcoxon rank sum tests, or one-way ANOVAs with Holm-Sidak corrected multiple comparison T tests. Calculated p values are displayed as *, p<0.05; **, p<0.01; ***, p<0.001; ****, p<0.0001. A p value of <0.05 was considered significant. For all graphs, the mean is represented by a horizontal line. When shown, error bars represent ±SEM. Experimental specific detailed statistic methods are described in corresponding figure legends and method sections.

Study Approval

Fresh tumor and peripheral blood were obtained from patients who gave institutional review board (IRB)-approved written informed consent prior to inclusion in the study (City of Hope IRB 05091, IRB 07047, and IRB 14346).

Data Availability:

All the single-cell RNA-sequencing data and NanoString data are uploaded in a GEO database. All R scripts used in this publication are available in https://github.com/weihuaguo/TEXinERpBC

Abbreviations: breast cancer, BC; breast recurrence score, BRS; estrogen receptor, ER; gene set enrichment analysis, GSEA; HER2/neu over-expressed, HER2+; interferon-γ, IFNγ; Molecular Taxonomy of Breast Cancer International Consortium, METABRIC; non-cancerous breast tissue, NCBT; high Oncotype DX BRS, Onc_{DX}hi; intermediate Oncotype DX BRS, Onc_{DX}int; low Oncotype DX BRS, Onc_{DX}lo; peripheral blood mononuclear cells, PBMCs; exhausted T cell, T_{EX}; tumor infiltrating lymphocyte, TIL; tumor microenvironment, TME; triple negative breast cancer, TNBC; tumor negative tumor draining lymph node, T− LN; tumor positive tumor draining lymph node, T+LN

### Example 2

Beyond regulatory T cells, the role of CD4+ T cells in tumor immune surveillance is largely unclear. More generally, the association of tumor infiltrating T cells with survival in breast cancer patients differs depending on the subtype of breast cancer. (1). Example 1 shows the inventors' discovery of novel roles for CD8+ T cell subsets in breast cancer patient outcomes. (2, 3). As CD4+ tumor infiltrating T cells often represent the major of CD3+ T cells found in breast cancer patient tumor tissues, an improved understanding of their contribution to patient outcomes is greatly needed.

Conventional CD4+ T cells may play an important role in anti-tumor immunity via a number of mechanisms. By producing pro-survival cytokines, CD4+ helper T cells are capable of promoting anti-tumor CD8+ T cell activity. (4) Furthermore, CD4+ T cell derived cytokines may help in priming tumor localized dendritic cells for enhanced antigen presenting cell function. (5). In addition to acting as helper cells, CD4+ T cells are capable of cancer cell lysis themselves, with evidence for cytotoxicity via both Granzyme B and interferon-γ secretion. (6). Thus, CD4+ T cells play a critical role in facilitating anti-tumor immunity with the tumor microenvironment.

Recently, CD4+ T cells marked by expression of the ectoenzyme CD26 (dipeptidyl peptidase-4, DPP4) have been identified as a subset with unique anti-tumor potential. (7). In addition to enzymatic activity, CD26 has been shown to have co-stimulatory properties as a receptor that can potentiate T cell receptor signaling. (8). CD26^{high} CD4+ T cells have been shown to exhibit enhanced tissue trafficking capacity, effector cytokine production, and memory potential. (9). Here we dissect the phenotype and function of CD26+CD4+ T cells in human breast cancer patients. We demonstrate that CD26+CD4+ T cells are uniquely capable of infiltrating tumor tissues and exerting anti-tumor effector activity. Finally, we show that CD26+CD4+ T cells are associated with good prognosis in both triple negative (TNBC) and estrogen receptor positive (ER+) early-stage breast cancer patients.

Results

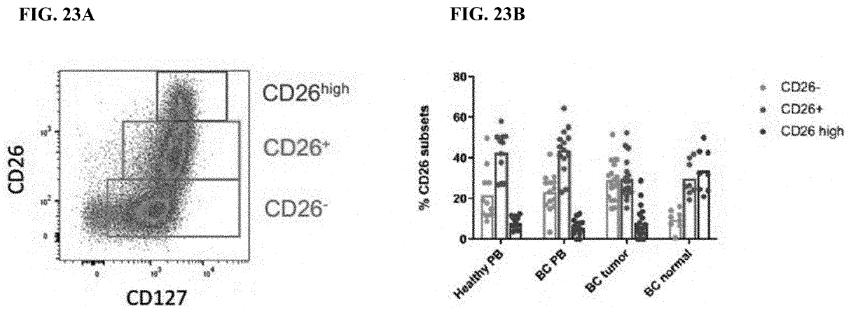

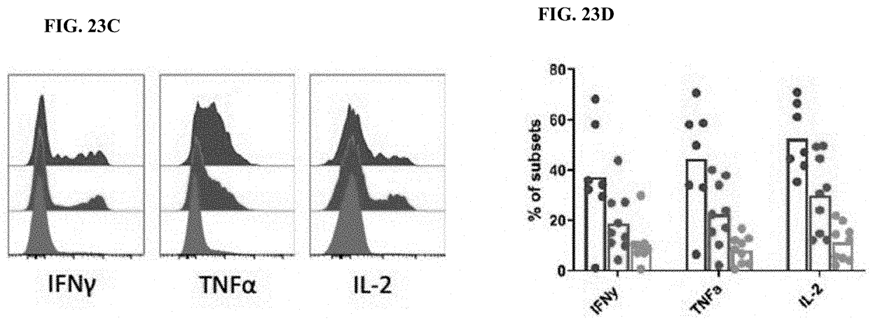

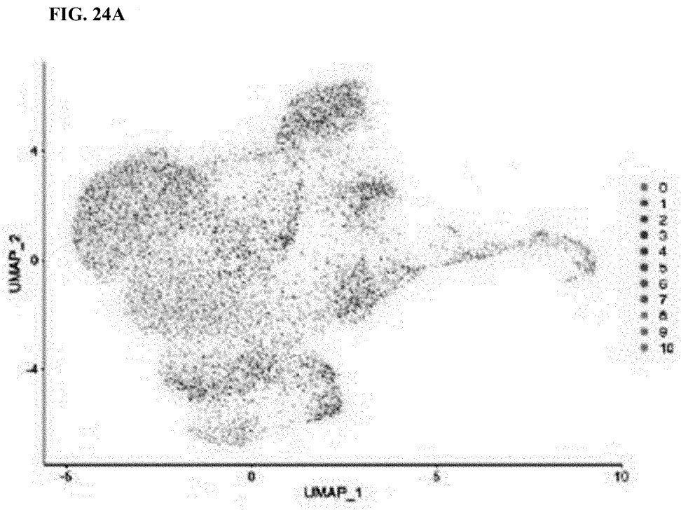

To investigate the presence and role of CD26+CD4+ T cells in breast cancer patient anti-tumor immunity, we first interrogated patient tissues for the presence and frequency of CD26-defined CD4+ T cell subsets. Peripheral blood, tumor tissue, and disease-free breast tissue were obtained from early-stage breast cancer patients undergoing surgical treatment. CD26 expression was examined by low cytometry on CD4+ T cells and found to strongly co-express with CD127 (IL-7R) expression (FIG. 23A). While CD26high expressing CD4+ T cells were found to be enriched in normal, disease-free breast tissues, increased fractions of CD26− CD4+ T cells were found in breast tumors, suggesting either tumor localized differentiation of CD4+ T cells (FIG. 23B). We next examined polyfunctionality of CD4+ T cell subsets based on expression of CD26. CD26high CD4+ T cells demonstrated increased capacity to express IFNγ, TNFα, and IL-2 in response to PMA/Ionomycin stimulation (FIGS. 23C-23D). Together these data identify CD26 expression to be linked with peripheral tissue infiltration and superior functionality.

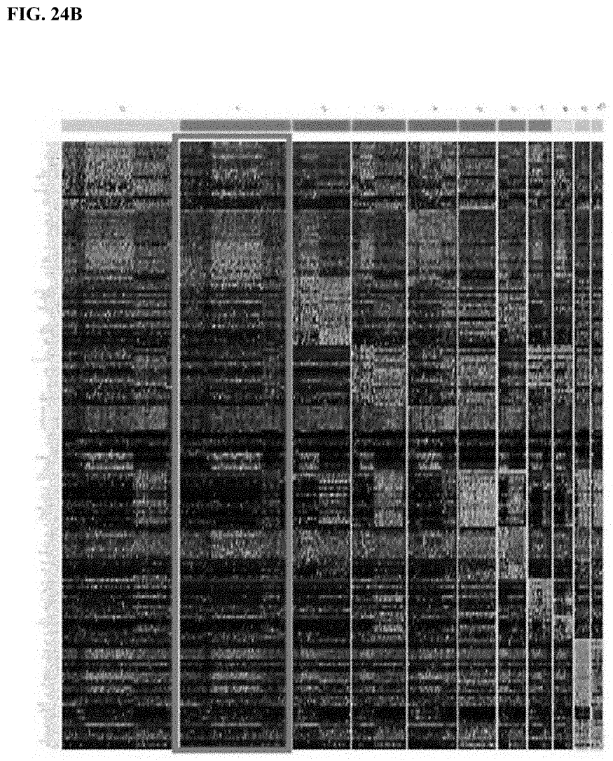

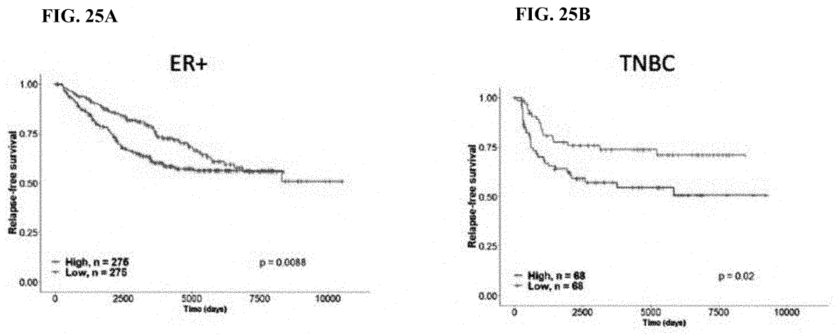

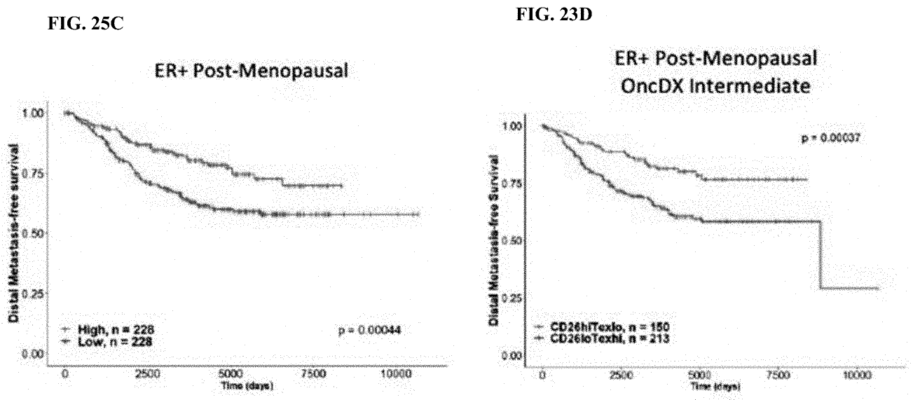

We next examined CD4+ T cells for unique gene expression profiles using a single cell RNA sequencing approach complemented by T cell receptor and CITE-seq protein expression analysis. Using an unbiased clustering approach we identified 11 unique CD4+ T cell subsets (FIG. 24A). Within these 11 subsets we identified a cluster enriched in CD26 (DPP4) gene expression and protein expression (FIG. 24B). Differential gene expression was then used to generate a unique 38-gene signature that identifies CD26+CD4+ T cells, as shown in Table 2. Analysis of the METABRIC breast tumor database found that the CD26 gene expression signature correlated with increased survival in both ER+ and TNBC patients (FIGS. 25A-25B). Furthermore, the CD26 gene expression signature strongly correlated with patient outcomes in ER+ postmenopausal breast cancer patients (FIG. 25C).

Table 1 and Example 1 describe the 25-gene signature of exhausted CD8+ T cells (Tex) that associates with both reduced overall survival and reduced time to relapse in ER+BC patients. The T_{EX} and CD26+CD4+ signatures do not overlap, demonstrating they are independent features of tumor biology. As such, these are independent and optionally adjunctive tests to predict patient outcome. In postmenopausal HR+BC patients, we find added predictive accuracy by combining assessment of our T_{EX} (higher in high-risk patients) and CD4+CD26+(higher in low-risk patients) signatures together. The combined T_{EX}:CD4+CD26+ signature accurately identifies Oncotype Dx® intermediate breast recurrence score patients with reduced survival (FIG. 25C). This demonstrates the potential value of T_{EX}:CD4+CD26+ signature assessment in supplementing the Oncotype Dx® BRST in standard of care treatment for postmenopausal HR+BC patients.

Methods

Human Samples

Tissues were obtained from consented BC patients undergoing standard of care therapy at City of Hope. Patient characteristics are summarized in Supplementary Table 1. Classification of tumor samples as ER+, PR+, or HER2+ was performed by clinical pathologists. NCBTs were composed of tissue from high-risk patient prophylactic mastectomies, contralateral breast from BC patient mastectomies, or tumor adjacent tissue. Due to limited cell numbers obtained from patient tumor samples, not all analyses shown were performed on the same samples. Tissue samples were provided by the City of Hope Biospecimen Repository. Other investigators may have received specimens from the same patients.

Sample Processing

Patient peripheral blood was obtained by venipuncture using heparin collection tubes, transported at room temperature from the clinic to the lab, and processed within 6 hours of drawing. PBMCs were isolated via Ficoll-Paque Separation (GE Healthcare) following the manufacturer's instructions. Solid tissue specimens were collected by surgical resection and collected in tubes containing cold HBSS (Life Technologies, Thermo Fisher Scientific) and transported on ice to the laboratory for processing within one hour of surgery. Tumor negative lymph nodes were mechanically dissociated and filtered into single cell suspensions. Tumor, tumor positive lymph nodes, and NCBT tissues were minced into pieces, mechanically dissociated with a gentleMACS Dissociator (Miltenyi Biotec), and enzymatically treated with 0.2 Wunsch units/ml Liberase™ (Roche) and 10 units/ml DNase (Sigma) in RPMI for up to 1 h as needed. If necessary, red blood cell (RBC) lysis was performed using RBC Lysis Buffer (Biolegend).

Flow Cytometry

Single cell suspensions were stained at RT in 2% FBS in PBS. For cytokine production assays, cells were stimulated with 50 ng/ml PMA (Sigma) and 1 μg/ml ionomycin (Sigma) in the presence of Golgi Plug (Biolegend) for 4 hours. Overnight fixation was performed as needed with IC Fixation Buffer (eBioscience). Fixation and permeabilization was performed with BD Cytofix/Cytoperm buffers for intracellular cytokine staining. Antibody cocktails were diluted in Brilliant Violet Buffer (BD Biosciences) when using two or more Brilliant Violet labeled antibodies. Samples were acquired using a BD Fortessa operating FACS Diva 6.1.3. Photomultiplier tube voltages were set using BD CS&T beads. Compensation was calculated using single stained OneComp compensation beads (eBioscience). Samples were stained with fluorescently tagged antibodies detailed in Supplementary Table 2. Antibodies were titrated for optimal signal to noise ratio prior to use. Flow cytometry analysis was performed using Flowjo v10.6. All samples were gated on single cells, lymphocytes, and CD3⁺ CD8⁺ populations (Supplemental FIG. 1). Histograms and zebra plots are used to display data.

Single Cell Sequencing and Analysis

Single-cell RNA sequencing was implemented through 10×chromium platform with recommended procedures. CellRanger was used to align sequence reads to human genome and count the aligned transcripts for each cell. The raw counts for each sample were directly filtered, normalized, and scaled by Seurat R package (v3.1.4) with the same parameters described above. The top 2,000 most variable genes were selected for sample integration and principle component analysis (PCA). All the tumor samples were integrated with standard integration workflow in Seurat (i.e., “FindIntegrationAnchors” and “IntegrateData” functions in Seurat with 50 dimensionality). PCA was implemented for the integrated data object. Based on the two heuristic methods in Seurat (modified Jack Straw procedure and ranking variance method), the top 20 principal components were used for the further non-linearly dimensional reduction (i.e., UMAP) and clustering analysis. Single cell sequencing data are deposited under the GEO accession number GSE.

Public Genomic Data Analysis

To evaluate the prognosis effect of CD26+ TILs in breast cancer, the Molecular Taxonomy of Breast Cancer International Consortium (METABRIC), one of the largest breast cancer multi-omics database, was downloaded with latest clinical information and normalized expression data (Illumina HT 12 platform) from European Genome-Phenome Archive (dataset ID: EGAD00010000210 and EGAD00010000211)(65, 66). A total 1,992 patients' data were obtained and organized. Based on the hormone receptor statuses, the ER+(all ER+ patients) and TNBC (ER− PR− HER2−) populations were stratified as 1098 and 269 patients respectively. The signature score of CD26+CD4+ T cells and other signatures was calculated using the sig.score function in genefu R package (v2.18.1) with default settings. The log 2 fold changes of genetic markers were considered as the weights in the signature. Based on the CD26 score, the ER+ and TNBC cohorts were stratified as described in figure legends and text. The survival comparison and Kaplan Meier curves between groups were implemented by survminer (v0.4.8) and survival (v3.2-7) R packages with log-rank statistics. Hazard ratios were generated with a Cox Proportional-Hazards model in univariate or multivariate plots. The multivariate Cox regression analysis accounted for influence of age, tumor grade, tumor size, and nodal status or as described.

The CIBERSORTx was utilized to deconvolute the METABRIC expression data to 22 major immune cell types (LM22 signature) with standard data pre-processing procedure and 500 permutations for significance analysis(70, 71). The relative abundances of all the 22 major immune cell types between T_{EX} ^{high} and T_{EX} low cohorts were compared by Wilcoxon singed-rank test. The differential expression analysis between T_{EX} ^{high} and T_{EX} low cohorts of METABRIC expression data was implemented by limma R packages (v3.42.2) and visualized by EnhancedVolcano R package (v1.4.0)(72). The Gene Set Enrichment Analysis was implemented by GSEA software (v4.0.3) with latest Hallmark Molecular Signatures Database from Broad Institute(73, 74).

The in-house Oncotype Dx® scores for all the METABRIC ER+ breast tumors (n=1098) were calculated using the “sig.score” function in genefu R package (v2.18.1) with default settings. The weights of genes in Oncotype Dx signature were calculated based on the Recurrence-Score algorithm described in Paik et al (ref). The standard Oncotype Dx® scores (0-100, Oncotype DX® Breast Recurrence Score) were calculated using the “oncotypedx” function in genefu R package (v2.18.1) with default settings(62). A strong correlation between in-house and standard Oncotype Dx® scores was observed (r=0.89, p<0.01) and the stratification criteria for standard Oncotype Dx® high and low groups were aligned with 85% (scaled score >25) and 15% (scaled score <15) percentiles of the whole population of ER+ breast cancer patients.

Mutiplex Immunofluorescence

Additional Statistics

Graphs and statistics were performed using Graphpad Prism 8 and specific R packages as described. Statistics described were generated using one-way unpaired student T tests, Wilcoxon rank sum tests, or one-way ANOVAs with Holm-Sidak corrected multiple comparison T tests. Calculated p values are displayed as *, p<0.05; **, p<0.01; ***, p<0.001; ****, p<0.0001. A p value of <0.05 was considered significant. For all graphs, the mean is represented by a horizontal line. When shown, error bars represent ±SEM. Experimental specific detailed statistic methods are described in corresponding figure legends and method sections.

Study Approval

Fresh tumor and peripheral blood were obtained from patients who gave institutional review board (IRB)-approved written informed consent prior to inclusion in the study (City of Hope IRB 05091, IRB 07047, and IRB 14346).

Data availability: All the single-cell RNA-sequencing data and NanoString data will be uploaded in a GEO database.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES FOR EXAMPLE 1

1. Fridman, Nat Rev Cancer 2012; 12:298-306.
2. Schnitt, Mod Pathol 2010; 23 Suppl 2:S60-4.
3. Stanton, Journal for ImmunoTherapy of Cancer 2016; 4(1). doi:10.1186/s40425-016-0165-6
4. Krishnamurti U, et al Human Pathology 2017; 64:7-12.
5. Miyoshi Y et al. Oncology Letters 2019; 17(2):2177-2186.
6. Ali H R et al. Ann Oncol 2014; 25:1536-1543.
7. Colleoni, Journal of Clinical Oncology 2016; 34(9):927-935.
8. Estrogen Receptor Status and the Future Burden of Invasive and In Situ Breast Cancers in the United States www.ncbi.nlm.nih.gov/pmc/articles/PMC4836802/. cited Apr. 30, 2020
9. Egelston C A et al. JCI Insight 2019; 4(19). doi:10.1172/jci.insight.130000
10. Savas, Nature Medicine 2018; 24(7):986-993.
11. Gruosso, Journal of Clinical Investigation 2019; 129(4):1785-1800.
12. McLane L M, et al Annual Review of Immunology 2019; 37(1):457-495.
13. Blackburn S D et al. Nat Immunol 2009; 10:29-37.
14. Zhou Q et al. Blood 2011; 117:4501-4510.
15. Ahmadzadeh M et al. Blood 2009; 114:1537-1544.
16. Gros A et al. Journal of Clinical Investigation 2014; 124(5):2246-2259.
17. Fourcade J et al. Journal of Experimental Medicine 2010; 207(10):2175-2186.
18. Egelston C A et al. Nature Communications; doi:10.1038/s41467-018-06653-9
19. Gupta P K et al. PLoS Pathog 2015; 11:e1005177.
20. Canale F P et al. Cancer Res 2018; 78:115-128.
21. Thommen D S et al. Nature Medicine 2018; 24(7):994-1004.
22. Duhen T et al. Nat Commun 2018; 9:2724.
23. Ahmadzadeh M et al. Blood 2009; 114:1537-1544.
24. Thommen D S et al. Nat Med 2018; 24:994-1004.
25. Kaech S M et al. Nat Immunol 2003; 4:1191-1198.
26. Lang K S et al. Eur J Immunol 2005; 35:738-745.
27. Jones R B et al. J Exp Med 2008; 205:2763-2779.
28. Miller B C et al. Nature Immunology 2019; 20(3):326-336.
29. Bengsch B et al. Immunity 2018; 48:1029-1045 e5.
30. Angelosanto J M, et al. J Virol 2012; 86:8161-8170.
31. Mognol G P et al. PNAS USA 2017; 114(13):E2776-E2785.
32. Brinza L et al. Scientific Reports 2016; 6. doi:10.1038/srep37651
33. Szabo P A et al. Nature Communications 2019; 10(1):1-16.
34. Fergusson J R et al. Cell Reports 2014; 9(3):1075-1088.
35. Harari A, et al Journal of Virology 2009; 83(7):2862-2871.
36. Martinez G J et al. Immunity 2015; 42(2):265-278.
37. Wherry E J et al. Immunity 2007; 27:670-684.
38. Li H et al. Cell 2019; 176(4):775-789.e18.
39. Schietinger A et al. Immunity 2016; 45(2):389-401.
40. Danaher P et al. Journal for ImmunoTherapy of Cancer 2018; 6(1):63.
41. Curtis, Nature 2012; 486(7403):346-352.
42. Newman, Nature Biotechnology doi:10.1038/s41587-019-0114-2
43. Simoni Y et al. Nature 2018; 557:575-579.
44. Post A E M et al. Clinical Cancer Research 2018; 24(14):3397-3408.
45. Paik et al., New England Journal of Medicine 2004; 351(27):2817-2826.
46. Cheng R, et al Frontiers in Oncology 2020; 10:1583.
47. Bates J P, et al BMC Cancer 2018; 18(1). doi:10.1186/s12885-018-4441-3
48. Goodman A M et al. Molecular Cancer Therapeutics 2017; 16(11):2598-2608.
49. Chabanon, Clinical Cancer Research 2016; 22(17):4309-4321.
50. McGranahan, Science 2016; 351(6280):1463-1469.
51. Wagner, Cell 2019; 177(5):1330-1345.e18.
52. Thorsson, Immunity 2018; 48(4):812-830.e14.
53. Carey, Clin Cancer Res 2007; 13:2329-2334.
54. Dimitrakakis, Breast Cancer Research 2009; 11(5). doi:10.1186/bcr2413
55. Dunn, Nature Reviews Immunology 2006; 6(11):836-848.
56. de Marchi T, Scientific Reports 2017; 7(1). doi:10.1038/s41598-017-02296-w
57. Huang, PLoS ONE 2014; 9(4):e94226.
58. Magkou C et al. Histopathology 2012; 60(7):1125-1132.
59. Hou Y et al. Journal of Cellular and Molecular Medicine 2018; 22(12):6077-6086.
60. Choi H J et al. Breast Cancer Research 2015; 17(1):1-23.
61. Harbeck N et al. The Lancet 2017; 389(10074):1134-1150.
62. Sparano J A et al. New England Journal of Medicine 2018; 379(2):111-121.
63. Rugo H S et al. Clinical Cancer Research 2018; 24(12):2804-2811.
64. Li W V, et al Nature communications 2018; 9(1):997.
65. Stuart T et al. Cell 2019; 177(7):1888-1902.e21.
66. Curtis C et al. Nature 2012; 486(7403):346-352.
67. Rueda O M et al. Nature 2019; 567(7748):399-404.
68. Haibe-Kains B et al. Journal of the National Cancer Institute 2012; 104(4):311-25.
69. Yang B et al. npj Breast Cancer 2018; 4(1):35.
70. Liberzon A et al. Cell Systems 2015; 1(6):417-425.
71. Newman A M et al. Nature Biotechnology; doi:10.1038/s41587-019-0114-2
72. Newman A M et al. Nature methods 2015; 12(5):453-7.
73. Ritchie M E et al. Nucleic Acids Research 2015; 43(7):e47.
74. Subramanian A et al. PNAS USA 2005; 102(43):15545-15550.
75. Liberzon A et al. Cell Systems 2015; 1(6):417-425.

### REFERENCES FOR EXAMPLE 2

1. Ali, H. R. et al. Ann. Oncol. Off. J. Eur. Soc. Med. Oncol. 25, 1536-1543 (2014).
2. Egelston, C. A. et al. JCI Insight 4, 130000 (2019).
3. Savas, P. et al. Nat. Med. 24, 986-993 (2018).
4. Nishimura, T. et al. J. Exp. Med. 190, 617-627 (1999).
5. Hor, J. L. et al. Immunity 43, 554-565 (2015).
6. Quezada, S. A. et al. J. Exp. Med. 207, 637-650 (2010).
7. Nelson, M. H. et al. Sci. Adv. (2020) doi:10.1126/sciadv.aba7443.
8. Dang, N. H., et al Cell. Immunol. 125, 42-57 (1990).
9. Bailey, S. R. et al. Nat. Commun. 8, 1961 (2017).

1. (canceled)

2. A method of detecting an elevated level of exhausted CD8+ T cells in a breast cancer patient, the method comprising detecting an elevated level of exhausted CD8+ T cells, relative to a standard control, in a biological sample obtained from the breast cancer patient.

3. The method of claim 2, further comprising administering to the patient an effective amount of a chemotherapeutic therapy.

4. (canceled)

5. A method of treating breast cancer in a patient in need thereof, the method comprising:
(i) detecting an elevated level of exhausted CD8+ T cells, relative to a standard control, in a biological sample obtained from the patient; and
(ii) administering to the patient an effective amount of a chemotherapeutic therapy.

6. The method of claim 2, wherein the exhausted CD8+ T cells are PD-1⁺ and CD39⁺.

7. The method of claim 2, wherein the elevated level of exhausted CD8+ T cells comprises an unequally weighted average of the elevated gene expression levels of the genes in Table 1.

8. The method of claim 2, wherein the breast cancer is estrogen receptor positive breast cancer.

9. The method of claim 2, wherein the patient is a premenopausal woman.

10. The method of claim 2, wherein the patient has an elevated level of exhausted CD8+ T cells relative to a standard control and a decreased level of CD26+CD4+ T cells relative to a standard control.

11. The method of claim 2, further comprising detecting a decreased level of CD26+CD4+ T cells in a biological sample obtained from the patient.

12. The method of claim 10, wherein the elevated level of exhausted CD8+ T cells comprises an unequally weighted average of the elevated gene expression levels of the genes in Table 1; and wherein the decreased level of CD26+CD4+ T cells comprises an unequally weighted average of the decreased gene expression levels of the genes in Table 2.

13. The method of claim 10, wherein the breast cancer is: (i) estrogen receptor positive breast cancer; (ii) progesterone receptor positive breast cancer; or (iii) estrogen receptor positive breast cancer and progesterone receptor positive breast cancer.

14-18. (canceled)

19. A method of detecting CD26+CD4+ T cells in a breast cancer patient, the method comprising detecting:
(i) a decreased level of CD26+CD4+ T cells, relative to a control, in a biological sample obtained from the breast cancer patient; or
(ii) an increased level of CD26+CD4+ T cells, relative to a standard control, in a biological sample obtained from the breast cancer patient.

20. The method of claim 19, wherein:
(i) further comprises administering to the patient an effective amount of a chemotherapeutic therapy; and
(ii) further comprises administering to the patient an effective amount of a non-chemotherapeutic therapy.

21. (canceled)

22. (canceled)

23. The method of claim 19, wherein the decreased level of CD26+CD4+ T cells comprises an unequally weighted average of the decreased gene expression levels of the genes in Table 2; and wherein the increased level of CD26+CD4+ T cells comprises an unequally weighted average of the increased gene expression levels of the genes in Table 2.

24-29. (canceled)

30. The method of claim 19, wherein the breast cancer is estrogen receptor positive breast cancer or triple negative breast cancer.

31-33. (canceled)

34. The method of claim 2, wherein the level is an mRNA expression level or a protein expression level.

35. (canceled)

36. The method of claim 2, wherein the biological sample is a tumor.

37. The method of claim 2, wherein a biological sample obtained from the patient has an intermediate gene expression level or a low gene expression level of Ki67, STK15, BIRC5, CCNB1, MYBL2, MMP11, CTSL2, GRB7, HER2, ER, PGR, BCL2, SCUBE2, GSTM1, BAG1, and CD68, relative to a standard control.

38-42. (canceled)

43. The method of claim 5, wherein the chemotherapeutic therapy comprises a chemotherapeutic agent selected from the group consisting of an alkylating agent, an antimetabolite compound, an anthracycline compound, an antitumor antibiotic, a platinum compound, a topoisomerase inhibitor, a vinca alkaloid, a taxane compound, an epothilone compound, or a combination of two or more thereof.

44. The method of claim 43, wherein the alkylating agent is carboplatin, chlorambucil, cyclophosphamide, melphalan, mechlorethamine, procarbazine, or thiotepa; the antimetabolite compound is azacitidine, capecitabine, cytarabine, gemcitabine, doxifluridine, hydroxyurea, methotrexate, pemetrexed, 6-thioguanine, 5-fluorouracil, or 6-mercaptopurine; the anthracycline compound is daunorubicin, doxorubicin, idarubicin, epirubicin, or mitoxantrone; the antitumor antibiotic is actinomycin, bleomycin, mitomycin, or valrubicin; the platinum compound is cisplatin or oxaliplatin; the topoisomerase inhibitor is irinotecan, topotecan, amsacrine, etoposide, teniposide, or eribulin; the vinca alkaloid is vincristine, vinblastine, vinorelbine, or vindesine; the taxane compound is paclitaxel or docetaxel; and the epothilone compound is epothilone, ixabepilone, patupilone, or sagopilone.

45-51. (canceled)
